(19) United States
(12) Patent Application Publication
(10) Pub. No.: US 2024/0218312 A1
(43) Pub. Date: Jul. 4, 2024

(54) CONTACTLESS ACOUSTOFLUIDIC SAMPLE AGITATOR AND USES THEREOF

(71) Applicant: CORNELL UNIVERSITY, Ithaca, NY (US)

(72) Inventors: Amir Mokhtare, Ithaca, NY (US); Gianpiero D. Palermo, New York, NY (US); Alireza Abbaspourrad, Ithaca, NY (US)

(21) Appl. No.: 18/561,592

(22) PCT Filed: May 18, 2022
(86) PCT No.: PCT/US2022/029844

§ 371 Date: Nov. 16, 2023

Related U.S. Application Data
(60) Provisional application No. 63/190,010, filed on May 18, 2021.

(51) Int. Cl. C12M 1/42; C12M 1/36; C12M 3/06

(52) U.S. Cl. CPC C12M 35/04; C12M 23/16; C12M 41/48

(57) ABSTRACT

Described herein are contactless acoustofluidic agitators and devices and systems thereof. In some embodiments, the acoustofluidic agitators and devices and/or systems thereof are configured as a microscale chip device. Also described herein are methods of using the acoustofluidic agitators and devices and/or systems thereof for manipulation of various cells, particles and/or other materials present in a liquid sample.

### CROSS-REFERENCE TO RELATED APPLICATIONS

This application claims the benefit of and priority to co-pending U.S. Provisional Patent Application No. 63/190,010, filed on May 18, 2021, entitled CONTACTLESS ACOUSTOFLUIDIC SAMPLE AGITATOR AND USES THEREOF,” the contents of which is incorporated by reference herein in its entirety.

### TECHNICAL FIELD

The subject matter disclosed herein is generally directed to acoustofluidic devices and systems, such as microfluidic devices and system, configured for cell processing and/or manipulations.

### BACKGROUND

Microscale devices and chips for processing tissues, cells, and other particles and materials has opened the door to high-throughput and automated processing techniques. However, current devices and/or techniques are not well suited for particular processes and/or cell, particles or material types. For example, some cells are susceptible to physical manipulation and current devices relying on physical processing means that contact the cell may reduce the viability or other functionality of cells processed by such devices. As such, there exists a need for improved devices, particularly, microscale devices suitable for e.g., cell processing, particularly those cells that are sensitive to physical manipulation.

Citation or identification of any document in this application is not an admission that such a document is available as prior art to the present invention.

### SUMMARY

Described in certain example embodiments herein are acoustofluidic contactless sample agitators comprising a piezoelectric substrate capable of propagating a surface acoustic wave; a chamber configured to contain a volume of a liquid, the chamber comprising one or more walls forming the sides of the chamber and a bottom comprising an opening; at least two pairs of orthogonal interdigitated transducers (IDTs) deposited on a surface of the piezoelectric substrate; and wherein the piezoelectric substrate is coupled to the chamber such that the two pairs of orthogonal IDTs are arranged about the periphery of the opening on the bottom of the chamber such that each IDT in each IDT pair is opposite each other on different sides of the opening, that the two IDT pairs are substantially perpendicular to each other about the periphery of the opening, and that the opening is substantially centered between the two IDT pairs, and wherein, together, the two pairs of IDTs are configured to produce tunable orthogonal surface acoustic waves in the piezoelectric substrate effective to produce acoustic streaming and/or acoustic radiation force within a liquid present in the chamber sufficient to mix the liquid present inside the chamber and/or agitate one or more particles, cells, and/or complexes thereof present in the liquid without trapping the one or more particles, cells, and/or complexes thereof within the liquid.

Described in certain example embodiments herein are sample processing systems comprising: (a) an acoustofluidic agitator as in any one of any one of the preceding paragraphs or as described elsewhere herein or a microfluidic device as in any one of as in any one of any one of the preceding paragraphs or as described elsewhere herein; and (b) one or more additional devices and/or systems capable of additional upstream or downstream processing, analyzing, manipulating, and/or storage of one or more particles, cells, and/or complexes thereof in the sample, wherein the one or more additional devices and/or systems are fluidically coupled with, electrically coupled with, and/or is in fluidic, electrical, optical, and/or wireless communication the acoustofluidic device or microfluidic device of (a).

Described in certain example embodiments herein are methods of separating cells from a complex of cells comprising exposing a sample comprising complexes of cells present in the chamber of an acoustofluidic device as in any one of any one of the preceding paragraphs or as described elsewhere herein, a microfluidic device as in any one of any one of the preceding paragraphs or as described elsewhere herein, or of a system as in any one of any one of the preceding paragraphs or as described elsewhere herein, to acoustic streaming and/or acoustic radiation force in the chamber by applying tunable orthogonal surface acoustic waves produced by the acoustofluidic device to the chamber, wherein the acoustic streaming produced in the chamber separates one or more cells and/or cell types from the complex of cells.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1E—SAW based oocyte denudation module (FIG. 1A) schematic depicting the “dry” state (left) and “wet” state (right) modes of denudation module including the configuration of IDTs used as SAW generators, and a microwell that is located at the center of IDTs. (FIG. 1B) The photos of fabricated “dry” state device (top) and “wet” state device with IDTs deposited on a X-cut LiNbO3 substrate. Disposable microwells shown in both photos can be reversibly bonded to the substrate for performing denudation procedure, X and Y shows the crystallographic axes of the substrate. (FIG. 1C) Side view of the “dry” state denudation module showing the configuration of IDTs in the periphery of the microwell located on top of the substrate and interaction of the Traveling SAW with the fluid and cells inside the microwell. The Rayleigh waves interacting with fluid convert into leaky Rayleigh waves that radiate into the liquid at the angle of θₜ. (FIG. 1D) Side view of the “wet” state denudation module showing the configuration of IDTs submerged inside microwell located on top of the substrate and interaction of the SAW with the fluid and cells inside the microwell. The Rayleigh waves are both directly coupled with liquid and convert into leaky Rayleigh waves that radiate into the liquid at the angle of θₜ. (FIG. 1E) Driving signal modalities employed for SAW generation. (i) Continuous wave (CW) (ii) Frequency modulated (FM) (iii) Pulse modulation (PM) and (iv) swept frequency modulation (SFM). Modulated signal modes switch the directions of dominant SAW propagating into the liquid at specified time intervals for achieving a complete agitation.

FIG. 2A-2E—Mechanisms of particle and cell agitation. Representative particle/cell trajectory measurements for CW and SFM driving signals using 15 and 90 micron particles. (FIG. 2A) The overlaid xy plane image of particle trajectories, generated from particle tracking studies and stacked top view images when both devices (dry state (i and ii) and wet state (iii)) were actuated with a 20 dBm CW signal at x-direction resonant frequency. (FIG. 2B) Overlaid xy plane image of particle trajectories, generated from particle tracking studies and stacked top view images when both devices (dry state (i and ii) and wet state (iii)) were actuated with a 20 dBm SFM signal at 1 second intervals. (FIG. 2C) LDV measured data: SAW standing mode and uniformity switching between the x direction (i) and y direction (ii). X and Y show the crystallographic axes of the substrate (FIG. 2D) cross-section and top-down schematic illustration of internal streaming in the liquid filled microwells of (i) the dry state device for CW actuation and (ii) SFM actuation mode; (FIG. 2E) schematic illustration of internal streaming in the liquid filled microwells of (i) the wet state device for CW actuation and (ii) SFM actuation.

FIG. 3A-3D—Displacement of the SAW on a bare LiNbO₃ substrate (no microwell on top) (FIG. 3A) on the dry state device (FIG. 3C) on the wet state device. Acoustic wave intensity characterization using the LDV measured displacements for (FIG. 3B) the dry state device (FIG. 3D) the wet state device.

FIG. 4A-4B—(FIG. 4A) Mean±s.e.m temperature variation from room temperature of the acoustic cavities (see figure inset) of the dry and wet state devices without a microwell as a function of input power (n=5 measurements), the dashed squares show the measuring areas; (FIG. 4B) ultrasound thermal effects, i.e., the difference in temperature after ultrasound stimulation and biological temperature (37° C.) on the M2 medium filling the microwell for both 2.2 Vrms SFM and PM driving modes. Figure inset shows the time constant at which the temperature reaches 67% of the stable temperature after 60 seconds.

FIG. 5A-5C—(FIG. 5A) Denudation efficiency as a function of device mode, frequency and driving signal modulation. (FIG. 5B) A compact cluster of MII stage COCs before any chemical (HA) or mechanical treatment. (FIG. 5C) A few completely or partially denuded oocytes retrieved from a dry state device operating at 80 MHz with a 20 dBm and SFM signal.

FIG. 6A-6C—(FIG. 6A) The effect of SAW power on denudation efficiency for both devices using an SFM signal and 60 second exposure period. There exists a significant correlation between the power level and denudation efficiency for both dry state (P value<0.05) and wet state (P value<0.05) modules. (FIG. 6B) The effect of exposure time on denudation efficiency of both devices using a 22 dBm SFM signal. There exists a significant correlation between the power level and denudation efficiency for both dry state (P value<0.05) and wet state (P value<0.05) modules. There is no significant difference between dry and wet state modules for performing denudation given the current operating conditions. (FIG. 6C) Representative images of partially denuded (i-iii) and completely denuded (iv) COCs using the ultrasound devices.

FIG. 7—Overall ICSI micromanipulation survival and embryo development data (mean and SD). Control is the manual protocol (MP). *P value>0.1 and **P value>0.1 indicate a comparable blastocyst rate between the control, dry state and wet state modules.

FIG. 8A-8B—Schematics of experimental design. (FIG. 8A) in vitro testing protocol for device performance evaluation and parameter optimization (FIG. 8B) in vivo testing protocol for experimental evaluation of the safety of the devices on the development of embryos.

FIG. 9A-9D—Image Segmentation results: FIG. 9A, image(i)-FIG. 9D, image(i) Original images taken with a phase-contrast microscope. FIG. 9A, image(ii)-FIG. 9D, image(ii) Segmented binary images capturing the darker ROI areas (than the background). FIG. 9A, image (iii)-FIG. 9D, image(iii) Segmented binary images capturing brighter ROI area. FIG. 9A, image(iv)-FIG. 9D, image(iv) final merged masks indicating the total cumulus area surrounding the oocyte. The yellow color (represented in greyscale) shows the overlap between the dark and bright masks. Scale bar in A is 150 microns and Scale bars in B-D are 25 microns.

FIG. 10A-10D—(FIG. 10A) Fabrication process of the microwell parts. (i-ii) shows the PMMA parts and their assembly like Legos (iii-v) shows the soft lithography process. (vi) Microwells with different contact area geometry have a hydraulic diameter of 2-2.5 mm and a height of 3 mm (FIG. 10B) fabrication process of SAW devices. (i-iii) shows IDT and electrode patterning on a lithium niobate substrate. (iv-v) shows mounting of the dies on PCB and wire bond securing using epoxy. (FIG. 10C) (i) top view image of microwell parts (ii) perspective view of the assembled microwell parts. (iii) The final PDMS microwells for the “dry” and “wet” configurations. (FIG. 10D) The perspective and close up images of assembled (i-ii) “dry” and (iii-iv) “wet” state devices.

FIG. 11A-11B—(FIG. 11A) S21 parameter shifts due to temperature increase by the attached heater below the substrate. (FIG. 11B) S21 parameters for x-direction with reflector gratings and y-direction without reflectors. The reflectors in the x-direction result in a narrower bandwidth compared to the y-direction allowing switching of the wave propagation with slight frequency change without using RF switches.

FIG. 12A-12C—(FIG. 12A) One-micron particle assembly at the bottom of the microwell in a “dry” state device when driven with a CW signal at 23 dBm using deionized water. (FIG. 12B) One-micron particle assembly at the bottom of the microwell in a “dry” state device when driven with a CW signal at 23 dBm using an M2 culture medium. (FIG. 12C) Driving “wet” state device at off-resonant frequencies at a higher power of 30 dBm using either of the deionized water of medium without a one-micron particle assembly.

FIG. 13A-13C—(FIG. 13A) Twin images as a result of lithium niobate crystal birefringence. (FIG. 13B) Light intensity decrease and twin image elimination by using a polarizer. (FIG. 13C) Polarizer placement on either top of the sample or before the camera in imaging setup eliminates the twin image. The amount of reduced light intensity can be compensated by increasing the illumination.

FIG. 14A-14D—Images of the surface acoustic denudation modules (FIG. 14A) on chip surface acoustic waves generator chip with resonant frequency of 80 MHz (FIG. 14B) close up picture of Lithium Niobate substrate with the patterned interdigitated transducer of the 80 Mhz device (FIG. 14C) on chip surface acoustic wave generator chip with resonant frequency of 197.3 MHz (FIG. 14D) close up picture of the Lithium Niobate substrate with the patterned IDTs of the 200 MHz device.

FIG. 15A-15C—(FIG. 15A-15B) top view of the ultrasonic device and (FIG. 15C) the device operation under a stereomicroscope.

FIG. 16A-16B—(FIG. 16A) a droplet containing HA exposed COCs before denudation and (FIG. 16B) completely denuded oocytes inside the droplet after 20 seconds continuous ultrasonic acoustic exposure at 79.2 MHz.

FIG. 17A-17B—Show image captures from movies demonstrating the working mechanism of the dry state 80 MHz device (FIG. 17A) in vitro as compared to in vivo, 200 MHz in SFM mode (FIG. 17B). The top left image of FIG. 17A shows the first 10 seconds of the working device. The top right image of FIG. 17A shows the second 10 seconds (from second 10-20) of the working device. The bottom left image of FIG. 17A shows the third 10 seconds (from second 20-30) of the working device. The bottom right image of FIG. 17A shows the fourth 10 seconds (from second 30-40) of the working device. During the 40 seconds of operation, the device is able to reach to almost complete denudation of the frozen cell.

FIG. 18A-8B—Shows image captures from movies of operation of the 80 MHz and 200 MHz devices tested using fresh COCs and performed by an experienced embryologist that had no prior experience working with the device.

FIG. 19—Shows images of an oocyte during preparation.

FIG. 20—Shows entire development of both control and ultrasound exposed zygotes side by side.

FIG. 21A-21E—Demonstrates results from overall embryo development. The developments of ultrasound exposed zygotes into 2-cell, 4-cell, morula and blastocyst are comparable to control zygotes.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y‘, and ‘greater than z’. In addition, the phrase “about ‘x’ to ‘y”’, where ‘x’ and ‘y’ are numerical values, includes “about ‘x’ to about ‘y”’.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

General discussion of microfluidics and microfluidic devices, fabrication thereof, and terms, can be found e.g., in Microfluidics: Fundamentals, Devices, and Applications (Y. Song (Ed.), D. Cheng (Ed.) and L. Zhao (Ed.) (Wiley) ISBN 978-3-527-80065-0 (2018); Manz et al. Microfluidics and Lab-on-a-chip. 2020 ISBN 978-1-78262-833-0; and Niculescu, A.-G.; Chircov, C.; Bîrc\check{ }a, A. C.; Grumezescu, A. M. Fabrication and Applications of Microfluidic Devices: A Review. Int. J. Mol. Sci. 2021, 22, 2011. https://doi.org/10.3390/ijms22042011.

A general discussion of reproductive anatomy and physiology and in vitro fertilization can be found in Knobil and Neill's Physiology of Reproduction 4th Edition (Plant and Zeleznik (Ed.) Academic Press. 2014. ISBN 9780123971753; Senger, P. L. Pathways To Pregnancy And Parturition (3^{rd}. Ed.). 2015. Current Conceptions Inc. ISBN 0965764834.

As used herein, the singular forms “a” “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Exemplary non-human animals include, without limitation, bovine, equine, ovine, porcine, camelid, canine, feline, and/or the like. Tissues, cells and their progeny of a biological entity obtained in vivo or cultured in vitro are also encompassed.

The term “biocompatible”, as used herein, refers to a substance or object that performs its desired function when introduced into an organism without inducing significant inflammatory response, immunogenicity, or cytotoxicity to native cells, tissues, or organs, or to cells, tissues, or organs introduced with the substance or object. For example, a biocompatible product is a product that performs its desired function when introduced into an organism without inducing significant inflammatory response, immunogenicity, or cytotoxicity to native cells, tissues, or organs.

Biocompatibility, as used herein, can be quantified using the following in vivo biocompatibility assay. A material or product is considered biocompatible if it produces, in a test of biocompatibility related to immune system reaction, less than 50%, 45%, 40%, 35%, 30%, 25%, 20%1, 5%1, 0%, 8%, 6%, 5%, 4%, 3%, 2%, or 1% of the reaction, in the same test of biocompatibility, produced by a material or product the same as the test material or product except for a lack of the surface modification on the test material or product. Examples of useful biocompatibility tests include measuring and assessing cytotoxicity in cell culture, inflammatory response after implantation (such as by fluorescence detection of cathepsin activity), and immune system cells recruited to implant (for example, macrophages and neutrophils).

As used herein, “cell type” refers to the more permanent aspects (e.g., a hepatocyte typically can't on its own turn into a neuron) of a cell's identity. Cell type can be thought of as the permanent characteristic profile or phenotype of a cell. Cell types are often organized in a hierarchical taxonomy, types may be further divided into finer subtypes; such taxonomies are often related to a cell fate map, which reflect key steps in differentiation or other points along a development process. Wagner et al., 2016. Nat Biotechnol. 34(111): 1145-1160.

As used herein, “coating” refers to any temporary, semi-permanent or permanent layer, covering or surface. A coating can be applied as a gas, vapor, liquid, paste, semi-solid, or solid. In addition, a coating can be applied as a liquid and solidified into a hard coating. Elasticity can be engineered into coatings to accommodate pliability, e.g., swelling or shrinkage, of the substrate or surface to be coated.

As used herein, “glass” refers to any type of glass including, but not limited to silicate glasses (e.g., soda-lime glass, borosilicate glass, lead glass, aluminosilicate glass, glass-ceramics, and fiber glass), silica-free glasses (e.g., amorphous metals and polymers), and molecular liquids and molten salts. Glasses can contain additives that can modify e.g., the optical properties (e.g., transparency, color, refractivity etc.), conductive properties or other properties of the glass.

As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, “polymer” refers to a chemical compound formed from a plurality of repeating structural units referred to as monomers “Polymers” are understood to include, but are not limited to, homopolymers, copolymers, such as for example, block, graft, random and alternating copolymers, terpolymers, etc. and blends and modifications thereof. Polymers can be formed by a polymerization reaction in which the plurality of structural units become covalently bonded together. When the monomer units forming the polymer all have the same chemical structure, the polymer is a homopolymer. When the polymer includes two or more monomer units having different chemical structures, the polymer is a copolymer.

As used interchangeably herein, “polymer blend” and “polymer mixture” refers to a macroscopically homogenous mixture of two or more different species of polymers. Unlike a copolymer, where the monomeric polymers are covalently linked, the constituents of a “polymer blend” and “polymer mixture” are separable by physical means and does not require covalent bonds to be broken. A “polymer blend” can have 2 or more (e.g., 2, 3, 4, 5, 6, 7, 8, 9, 10 or more) different polymer constituents.

As used herein, “separated” can refer to the state of being physically divided from the original source or population such that the separated compound, agent, particle, or molecule can no longer be considered part of the original source or population.

As used herein, “surface,” in the context herein, refers to a boundary of a product. The surface can be an interior surface (e.g., the interior boundary of a hollow product), or an exterior or outer boundary or a product. Generally, the surface of a product corresponds to the idealized surface of a three dimensional solid that is topological homeomorphic with the product. The surface can be an exterior surface or an interior surface. An exterior surface forms the outermost layer of a product or device. An interior surface surrounds an inner cavity of a product or device, such as the inner cavity of a tube. As an example, both the outside surface of a tube and the inside surface of a tube are part of the surface of the tube. However, internal surfaces of the product that are not in topological communication with the exterior surface, such as a tube with closed ends, can be excluded as the surface of a product. In some embodiments, an exterior surface of the product is chemically modified, e.g., a surface that can contact an immune system component. In some embodiments, where the product is porous or has holes in its mean (idealized or surface), the internal faces of passages and holes are not considered part of the surface of the product if its opening on the mean surface of the product is less than 1 m.

As used herein “contactless” refers to not requiring physical contact between to objects. For example, in the context of contactless agitation, mixing, or separation, “contactless” refers to achieving a result (agitating, mixing, and/or separation) without physical contact between a component (e.g., cell, particle, molecule, or other material) of a sample (e.g., a liquid sample) and a physical component (e.g., a wall, fin, paddle, or other solid or semi solid component) of the device.

As used herein “interdigitated transducer” or “interdigital transducer” (IDT) both refer to a device that is composed of comb shaped arrays of electrodes (typically metallic electrodes). IDTS herein can be planar, curved, or other suitable shape or configuration.

As used herein “tunable” means to be capable of being adjusted in one or more attributes to achieve particular purpose.

The term “trapping” used herein is a term of art that refers to keeping molecules, cells, particles or other components of a liquid in a fixed position within a space, such as a channel or microchannel.

As used interchangeably herein, the terms “sufficient” and “effective,” can refer to an amount (e.g., mass, volume, dosage, concentration, wavelength, frequency, and/or time period, or other relevant unit) needed to achieve one or more desired result(s).

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### OVERVIEW

Oocyte denudation is a crucial prerequisite for unequivocal evaluation of oocyte maturity and successful intracytoplasmic sperm injection. Residual cumulus cells can potentially prevent the oocyte from adequate manipulation and may represent a source of DNA contamination during trophectoderm biopsy for PCR-based preimplantation genetic testing. The established manual denuding method, developed decades ago, is composed of two steps of enzymatic digestion followed by mechanical stripping of cumulus using pipettes. Hyaluronidase is commonly used to break down the hyaluronic acid of the matrix encompassing the cumulus to facilitate their dispersion. It has been shown that high shear stresses during mechanical treatment can dislocate the polar body (PB) and meiotic spindle (MS) leading to a suboptimal intracytoplasmic sperm injection (ICSI) outcome and poor embryo development. On the other hand, although prolonged enzymatic exposures can minimize the time and shear stress of the mechanical procedure, It can expand the perivitelline space (PVS) and convolute the following micromanipulation. Despite its importance since the emergence of ICSI, manual pipetting for oocyte denudation has little changed over decades and still remains inefficient, labor-intensive and fatigues the embryologist. The procedure is highly skill dependent, time sensitive and suffers from intra operator variability which prevents its standardization.

In the context of on-chip technologies, female gamete processing, unlike semen processing, has rarely been scrutinized by novel microfluidic technologies. There are a few examples that suggest a microfluidic channel is capable compressing the cumulus to both sides of the oocyte and pulling these cells off of the individual oocytes at a right corner present in the channel. (Zeringue, H. C. & Beebe, D. J. Microfluidic removal of cumulus cells from Mammalian zygotes. in Germ Cell Protocols 365-373 (Springer, 2004) and Zeringue, H. C., Rutledge, J. J. & Beebe, D. J. Early mammalian embryo development depends on cumulus removal technique. Lab Chip 5, 86-90 (2005)). More recently, Weng et al. (Weng, L. et al. On-chip oocyte denudation from cumulus-oocyte complexes for assisted reproductive therapy. Lab Chip 18, 3892-3902 (2018)) developed a microfluidic chip that achieves multiple cumulus-oocyte-complexes (COCs) processing by physically shearing them against sharp corrugated walls. In their device, COCs are pushed against jagged side walls in a contraction section followed by an expansion section for reorientation. Applicant also recently showed that complete and controlled denudation is achievable in a non-contact oscillatory microfluidic channel that uses a bas-relief structure to twist the flow inside the channel. In our suggested platform the denudation extent is controlled by the fluid flow and frequency of oscillation (Mokhtare, A., Xie, P., Abbaspourrad, A., Rosenwaks, Z. & Palermo, G. Toward an ICSI chip: automated microfluidic oocyte denudation module. in HUMAN REPRODUCTION vol. 35 71-72 (OXFORD UNIV PRESS GREAT CLARENDON ST, OXFORD OX2 6DP, ENGLAND, 2020 and Mokhtare, A., Xie, P., Abbaspourrad, A., Rosenwaks, Z. & Palermo, G. D. EMBRYOLOGY LAB-ON-A-CHIP: AUTOMATED OOCYTE DENUDATION MICROFLUIDIC DEVICE. Fertil. Steril. 114, e76 (2020)). Despite the success and improvements, all methods acutely suffer from irreversibility of the chips. Oocytes are rare and precious cells, so any technology should allow their recoverability at any time of emergency. Current devices and techniques also require additional steps for preparation and delicate loading of the COCs to the devices which make the overall process prohibitively cumbersome and lengthy. In addition, constant monitoring of oocytes due the intrinsic two-dimensional structure of chips and limited field of view (FOV) of microscope is not possible.

As such improved techniques, devices, and/or systems for oocyte preparation for downstream manipulations and processes such as ICIS and IVF are needed.

With that said, embodiments disclosed herein can provide contactless acoustofluidic devices that can at least agitate complexes of cells present in a sample such that one or more cells or cell types can be separated from the complex without contacting the cells. Thus, the contactless acoustofluidic device can be capable of denudating COC to prepare oocytes for downstream manipulations such as ICIS. Generally, contactless agitation of the complexes is achieved by the devices described herein from an acoustic stream and/or Rayleigh wave scattering generated in the chamber containing the sample by tunable orthogonal surface acoustic waves propagated through the surface of a piezoelectric substrate stimulated by two pairs of orthogonal interdigitated transducers deposited on the surface of the piezoelectric substrate. The acoustofluidc device described herein can agitate/mix/and/or separate complexes of cells, particles, or other components in the liquid sample without trapping them in any one fixed location within the chamber. One advantage of the acoustofluidc device described herein is that in some embodiments, one or more of the components is configured for reuse.

It will be appreciated that while denudation of oocytes is one application for the devices described herein and that in view of the disclosure herein one of ordinary skill in the art will be able to adapt and tune the device to achieve agitation, mixing, and/or separation of cells, particles, or other components of a liquid sample in a contactless manner without trapping the contents of the liquid sample in a fixed location within the chamber of the acoustofluidc device described herein. Within this application COC denudation is provided as an example and the principles described in connection with COC denudation can be applied to adapt the specific configuration of the agitator, device, and/or system and/or its operation for use in other contexts as will be appreciated by one of ordinary skill in the art in view of the description provided herein.

Also described herein are devices, such as microfluidic devices, that can contain one or more of the acoustofluidc devices described herein. In some exemplary embodiments, the microfluidic devices include one or more microchannels, reservoirs, chambers, pumps, channels and the like in addition to the one or more acoustofluidc devices. In some embodiments, the acoustofluidic device and/or the microfluidic device is configured as a chip, such as a lab on a chip, that can be used as a stand-alone device or be coupled with one or more additional devices, systems, and the like.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Acoustofluidic Agitators, Devices, and Systems

Generally, described herein acoustofluidic devices that can provide contactless agitation of samples within a liquid (such as a droplet or larger volume) that can be tuned to provide sufficient agitation via acoustic streaming and/or acoustic radiation force generated within the liquid to separate one or more cell types from a complex of cells. The acoustofluidic devices can be incorporated into other devices (such as microfluidic devices) where they can be coupled to other components such as channels, reservoirs, electrical components (wires, receivers, transmitter, sensors), optical components (optical fibers, lenses, microscopes), and the like. The devices can be incorporated into systems that provide other processing and/or analytics manipulation or processing via the acoustofluidic agitator or device in which one or more acoustofluidic agitators are incorporated. The agitators, devices, and systems herein can be fabricated by any suitable method and/or technique. Such techniques will be appreciated by one of skill in the art and can include without limitation, lithography, molding, casting, various material deposition techniques, including thin film, chemical deposition, additive manufacturing techniques (e.g., 3D printing), and the like.

### Acoustofluidic Agitators

Described in certain example embodiments herein are acoustofluidic contactless sample agitators. In some embodiments, the acoustofluidic contactless sample agitator includes a piezoelectric substrate capable of propagating a surface acoustic wave; a chamber configured to contain a volume of a liquid, the chamber comprising one or more walls forming the sides of the chamber and a bottom comprising an opening; at least two pairs of orthogonal interdigitated transducers (IDTs) deposited on a surface of the piezoelectric substrate; and where the piezoelectric substrate is coupled to the chamber such that the two pairs of orthogonal IDTs are arranged about the periphery of the opening on the bottom of the chamber such that each IDT in each IDT pair is opposite each other on different sides of the opening, that the two IDT pairs are substantially perpendicular to each other about the periphery of the opening, and that the opening is substantially centered between the two IDT pairs, and where, together, the two pairs of IDTs are configured to produce and/or are capable of producing tunable orthogonal surface acoustic waves in the piezoelectric substrate effective to produce acoustic streaming and/or acoustic radiation force within a liquid present in the chamber sufficient to mix the liquid present inside the chamber and/or agitate one or more particles, cells, and/or complexes thereof present in the liquid without trapping the one or more particles, cells, and/or complexes thereof within the liquid.

In certain example embodiments, the chamber is detachably coupled to the piezoelectric substrate.

In certain example embodiments, the two pairs of orthogonal IDTs are arranged about the periphery of the opening such that they do not extend into the opening and do not contact a liquid present in the chamber.

In certain example embodiments, the two pairs of orthogonal IDTs are arranged about the periphery of the opening such that a portion of each IDT extends into the opening such that the portion contacts a liquid present in the chamber. In certain example embodiments, none of the extended portions of the IDTs touch one another. In certain example embodiments, the extended portions of the IDTs are covered with a shielding material. In certain example embodiments, the shielding material is a self-assembled monolayer film of trichlorosilane, silicon dioxide, or Teflon film.

In certain example embodiments, the piezoelectric substrate is a piezoelectric ceramic, a piezoelectric crystal, a piezoelectric thin film, or a combination thereof. In some embodiments, the piezoelectric ceramic is barium titanate, lead titanate, lead zirconate titanate, potassium niobate, lithium niobate, sodium tungstate. In some embodiments, the piezoelectric crystal is quartz, topaz, tourmaline, berlinite, gallium orthophosphate, or langasite. In some embodiments, the piezoelectric thin film is zinc oxide or aluminum nitride.

In some embodiments, one or more of the components are 0-100% optically transparent or translucent, with 0% being completely (or 100%) opaque and 100% being completely transparent or translucent. In some embodiments, one or more of the components is/are 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% transparent or translucent.

In certain example embodiments, the IDTs are planar IDTs. Exemplary planar IDTs are described elsewhere herein and will be appreciated by one of ordinary skill in the art in view of the description herein.

In certain example embodiments, the pair of IDTs forming an x-axis pair of the two pairs of orthogonal IDTs has a different configuration than the pair of IDTs forming a y-axis pair of the two pairs of orthogonal IDTs.

In certain example embodiments, the number of interleaving electrodes in each IDT of the x-axis pair of IDTs is the same, wherein the number of interleaving electrodes in each IDT of the y-axis pair of IDTs is the same, and wherein the number of interleaving electrodes in the each of the x-axis pair of IDTs is different than the number of interleaving electrodes in the y-axis pair of IDTs. The number of interleaving electrodes in each IDT can independently range from 2 to 10 or more, such as 2, 3, 4, 5, 6, 7, 8, 10, or more.

In certain example embodiments, the number of interleaving electrodes in each of the x-axis pair of IDTs is less than, is more than, or is equal to the number of interleaving electrodes in each of the y-axis pair of IDTs.

In certain example embodiments, the number of interleaving electrodes in each of the x-axis pair of IDTs and/or the number of interleaving electrodes in each of the y-axis pair of IDTs ranges from 1-100, 5-100, 10-100, 15-100, 20-100, 25-100, 30-100, 35-100, 40-100, 45-100, 50-100, 55-100, 60-100, 65-100, 70-100, 75-100, 80-100, 85-100, 90-100, or 95-100. In certain example embodiments, the number of interleaving electrodes in each of the x-axis pair of IDTs and/or the number of interleaving electrodes in each of the y-axis pair of IDTs is, 1,2,3,4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100. In certain example embodiments, the number of interleaving electrodes in each of the y-axis pair of IDTs is 20 and/or the number of interleaving electrodes in each of the x-axis pair of IDTs is 5.

In certain example embodiments, the spacing between each interleaving electrode of each IDT in the x-axis pair of IDTs is different than or the is the same as the spacing between each interleaving electrode of each IDT in the y-axis pair of IDTs, and wherein optionally the spacing between each interleaving electrode of each IDT in the x-axis pair, the y-axis pair, or both ranges from about 5 to about 150 microns, about 10 microns to about 150 microns, about 20 microns to about 150 microns, about 30 microns to about 150 microns, about 40 microns to about 150 microns, about 50 microns to about 150 microns, about 60 microns to about 150 microns, about 70 microns to about 150 microns, about 80 microns to about 150 microns, about 90 microns to about 150 microns, about 100 microns to about 150 microns, about 110 microns to about 150 microns, about 120 microns to about 150 microns, about 130 microns to about 150 microns, or about 140 microns to about 150 microns. In some embodiments the spacing between each interleaving electrode of each IDT in the x-axis pair, the y-axis pair, or both is about 5 microns, 6 microns, 7 microns, 8 microns, 9 microns, about 10 microns, 11 microns, 12 microns, 13 microns, 14 microns, 15 microns, 16 microns, 17 microns, 18 microns, 19 microns, 20 microns, 21 microns, 22 microns, 23 microns, 24 microns, 25 microns, 26 microns, 27 microns, 28 microns, 29 microns, 30 microns, 31 microns, 32 microns, 33 microns, 34 microns, 35 microns, 36 microns, 37 microns, 38 microns, 39 microns, 40 microns, 41 microns, 42 microns, 43 microns, 44 microns, 45 microns, 46 microns, 47 microns, 48 microns, 49 microns, 50 microns, 51 microns, 52 microns, 53 microns, 54 microns, 55 microns, 56 microns, 57 microns, 58 microns, 59 microns, 60 microns, 61 microns, 62 microns, 63 microns, 64 microns, 65 microns, 66 microns, 67 microns, 68 microns, 69 microns, 70 microns, 71 microns, 72 microns, 73 microns, 74 microns, 75 microns, 76 microns, 77 microns, 78 microns, 79 microns, 80 microns, 81 microns, 82 microns, 83 microns, 84 microns, 85 microns, 86 microns, 87 microns, 88 microns, 89 microns, 90 microns, 91 microns, 92 microns, 93 microns, 94 microns, 95 microns, 96 microns, 97 microns, 98 microns, 99 microns, 100 microns, 101 microns, 102 microns, 103 microns, 104 microns, 105 microns, 106 microns, 107 microns, 108 microns, 109 microns, 110 microns, 111 microns, 112 microns, 113 microns, 114 microns, 115 microns, 116 microns, 117 microns, 118 microns, 119 microns, 120 microns, 121 microns, 122 microns, 123 microns, 124 microns, 125 microns, 126 microns, 127 microns, 128 microns, 129 microns, 130 microns, 131 microns, 132 microns, 133 microns, 134 microns, 135 microns, 136 microns, 137 microns, 138 microns, 139 microns, 140 microns, 141 microns, 142 microns, 143 microns, 144 microns, 145 microns, 146 microns, 147 microns, 148 microns, 149 microns, or 150 microns. In certain example embodiments, the spacing of each IDT in the x-axis pair of IDTs is about 5 microns, the spacing of each IDT in the y-axis pair is about 20 microns, or both.

In some embodiments, the spacing is aligned or is a close to the average widest dimension (e.g., diameter or other appropriate dimension, such as length or width) as the cell type that is trying to be separated from a complex or otherwise singled out. For example, in the context of denudation of COC examples herein, 50 microns and 20 microns were chosen for 80 MHz and 200 MHz devices, respectively. Also, it will be appreciated that the speed of sound should be considered for selection of an appropriate spacing for a given application of the device. The speed of sound in the x and y directions are different, and the spacing should be adapted/selected accordingly.

In certain example embodiments, the aperture (width of the IDT/longest dimension of the IDT) is or ranges from about 10λ, 20λ, 30λ, 40λ, 50λ, 60λ, 70λ, 80λ, 90λ, 100λ, 110λ, 120λ, 130λ, 140λ, 150λ, 160λ, 170λ, 180λ, 190λ, 200λ, 210, 220λ, 230λ, 240λ, 250λ, 260λ, 270λ, 280λ, 290λ, 300λ, 310λ, 320λ, 330λ, 340λ, 350λ, 360λ, 370λ, 380λ, 390λ, 400λ, 410λ, 420λ, 430λ, 440λ, 450λ, 460λ, 470λ, 480λ, 490λ, 500λ, 510λ, 520λ, 530λ, 540λ, 550λ, 560λ, 570λ, 580λ, 590λ, 600λ, 610λ, 620λ, 630λ, 640λ, 650λ, 660 λ, 670λ, 680λ, 690λ, 700λ, 710λ, 720λ, 730λ, 740λ, 750λ, 760λ, 770λ, 780λ, 790λ, 800λ, 810λ, 820λ, 830λ, 840λ, 850λ, 860λ, 870, 880λ, 890λ, 900λ, 910λ, 920λ, 930λ, 940λ, 950λ, 960λ, 970λ, 980λ, 990λ, or/to about 1000λ.

The width and length of the IDTs are generally equal to the wavelength of the wave that they produce and the aperture length. For example, for an 80 MHz agitator (such as one provided in the Examples herein), the width (which is considered as the shorter dimension here) is equal to about 50 microns (which is the same as the wavelength in this frequency), and the length (which is the longer dimension here) is equal to 50*50 microns (Aperture). The aperture length can be adjusted independent of wavelength. However, the spacing of the IDT fingers depends on the piezoelectric material (speed of sound on that piezoelectric material) and the frequency of the sound wave (e.g., a SAW) that is desired. As such and using a 500 micron thick lithium niobate piezoelectric material (such as the one used in the Examples herein) a spacing of between 10-150 microns and aperture lengths of between 10λ and 1000λ can be used.

In certain example embodiments, the width, length, or both of each interleaving electrode of each IDT in the x-axis pair of IDTs is different than or is the same as the width and/or length of each interleaving electrode of each IDT in the y-axis pair of IDTs.

The acoustofluidic agitator can include one or more reflector gratings. In certain example embodiments, the acoustofluidic contactless sample agitator further comprises one or more reflector gratings, wherein one or more of the one or more reflector gratings backs one or more IDTs. In certain example embodiments, the acoustofluidic contactless sample agitator comprises two reflector gratings and wherein each of the two reflector gratings back a different IDT. In certain example embodiments, each of the two reflector gratings back a different IDT in the same pair of orthogonal IDTs. In certain example embodiments, the reflector gratings back each IDT in the orthogonal pair of IDTs forming an x-axis pair of IDTs. In some embodiments, reflector gratings are only included when straight IDTs are used. In certain example embodiments, the one or more reflector gratings comprise grating transducers comprising shortened electrodes, deposits of material on the piezoelectric material, such as periodic metal or other material strips deposited on the surface of the piezoelectric material. In certain example embodiments, the reflector gratings are configured such that unidirectional wave propagation is achieved. In some embodiments the number of gratings is sufficient to achieve unidirectional wave propagation. In some embodiments, the number of reflector gratings is or can range from 1 or/to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 or more. In certain example embodiments, the reflector gratings each have a reflection coefficient effective to achieve unidirectional wave propagation. Methods and techniques for measuring unidirectional wave propagation and thus determining reflector gratings with appropriate reflection coefficients effective to achieve unidirectional wave propagation.

In certain example embodiments, the acoustofluidic contactless sample agitator is configured to generate ultrasonic surface acoustic waves with frequencies within the therapeutic or diagnostic imaging range. In certain example embodiments, the acoustofluidic contactless sample agitator is configured to generate or generates during operation ultrasonic surface acoustic waves with frequencies that are or are ranging from about 2 MHz to about 200 MHz or more, from about 5 MHz, 10 MHz, 20 MHz, 30 MHz, 40 MHz, 50 MHz, 60 MHz, 70 MHz, 80 MHz, 90 MHz, 100 MHz, 110 MHz, 120 MHz, 130 MHz, 140 MHz, 150 MHz, 160 MHz, 170 MHz, 180 MHz, or about 190 MHz to about 200 MHz. In certain example embodiments, the acoustofluidic contactless sample agitator is configured to generate or generates during operation ultrasonic surface acoustic waves with frequencies that are about 1 MHz, 2 MHz, 3 MHz, 4 MHz, 5 MHz, 6 MHz, 7 MHz, 8 MHz, 9 MHz, 10 MHz, 11 MHz, 12 MHz, 13 MHz, 14 MHz, 15 MHz, 16 MHz, 17 MHz, 18 MHz, 19 MHz, 20 MHz, 21 MHz, 22 MHz, 23 MHz, 24 MHz, 25 MHz, 26 MHz, 27 MHz, 28 MHz, 29 MHz, 30 MHz, 31 MHz, 32 MHz, 33 MHz, 34 MHz, 35 MHz, 36 MHz, 37 MHz, 38 MHz, 39 MHz, 40 MHz, 41 MHz, 42 MHz, 43 MHz, 44 MHz, 45 MHz, 46 MHz, 47 MHz, 48 MHz, 49 MHz, 50 MHz, 51 MHz, 52 MHz, 53 MHz, 54 MHz, 55 MHz, 56 MHz, 57 MHz, 58 MHz, 59 MHz, 60 MHz, 61 MHz, 62 MHz, 63 MHz, 64 MHz, 65 MHz, 66 MHz, 67 MHz, 68 MHz, 69 MHz, 70 MHz, 71 MHz, 72 MHz, 73 MHz, 74 MHz, 75 MHz, 76 MHz, 77 MHz, 78 MHz, 79 MHz, 80 MHz, 81 MHz, 82 MHz, 83 MHz, 84 MHz, 85 MHz, 86 MHz, 87 MHz, 88 MHz, 89 MHz, 90 MHz, 91 MHz, 92 MHz, 93 MHz, 94 MHz, 95 MHz, 96 MHz, 97 MHz, 98 MHz, 99 MHz, 100 MHz, 101 MHz, 102 MHz, 103 MHz, 104 MHz, 105 MHz, 106 MHz, 107 MHz, 108 MHz, 109 MHz, 110 MHz, 111 MHz, 112 MHz, 113 MHz, 114 MHz, 115 MHz, 116 MHz, 117 MHz, 118 MHz, 119 MHz, 120 MHz, 121 MHz, 122 MHz, 123 MHz, 124 MHz, 125 MHz, 126 MHz, 127 MHz, 128 MHz, 129 MHz, 130 MHz, 131 MHz, 132 MHz, 133 MHz, 134 MHz, 135 MHz, 136 MHz, 137 MHz, 138 MHz, 139 MHz, 140 MHz, 141 MHz, 142 MHz, 143 MHz, 144 MHz, 145 MHz, 146 MHz, 147 MHz, 148 MHz, 149 MHz, 150 MHz, 151 MHz, 152 MHz, 153 MHz, 154 MHz, 155 MHz, 156 MHz, 157 MHz, 158 MHz, 159 MHz, 160 MHz, 161 MHz, 162 MHz, 163 MHz, 164 MHz, 165 MHz, 166 MHz, 167 MHz, 168 MHz, 169 MHz, 170 MHz, 171 MHz, 172 MHz, 173 MHz, 174 MHz, 175 MHz, 176 MHz, 177 MHz, 178 MHz, 179 MHz, 180 MHz, 181 MHz, 182 MHz, 183 MHz, 184 MHz, 185 MHz, 186 MHz, 187 MHz, 188 MHz, 189 MHz, 190 MHz, 191 MHz, 192 MHz, 193 MHz, 194 MHz, 195 MHz, 196 MHz, 197 MHz, 198 MHz, 199 MHz, or about 200 MHz.

In certain example embodiments, the acoustofluidic contactless sample agitator is configured to generate and propagate surface acoustic waves having a cell scale wavelength. In certain example embodiments, the wavelength is less than the diameter of an oocyte. In certain example embodiments, the wavelength is about the same as the average diameter of a cumulus cell or other cell complexed or associated with an oocyte, optionally a human oocyte, or non-human animal oocyte. In certain example embodiments, the wavelength is greater than zero but less than about 120 μm, 110 μm, 100 μm, 90 μm, 80 μm, 70 μm, 60 μm, 50 μm, 40 μm, 30 μm, 25 μm, 20 μm, 15 μm, 10 μm, or less than about 5 μm. In certain example embodiments, the wavelength is greater than zero but less than about 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 am, 22 μm, 23 μm, 24 μm, 25 μm, 26 μm, 27 μm, 28 μm, 29 μm, 30 μm, 31 μm, 32 μm, 33 am, 34 μm, 35 μm, 36 μm, 37 μm, 38 μm, 39 μm, 40 μm, 41 μm, 42 μm, 43 μm, 44 μm, 45 am, 46 μm, 47 μm, 48 μm, 49 μm, 50 μm, 51 μm, 52 μm, 53 μm, 54 μm, 55 μm, 56 μm, 57 am, 58 μm, 59 μm, 60 μm, 61 μm, 62 μm, 63 μm, 64 μm, 65 μm, 66 μm, 67 μm, 68 μm, 69 am, 70 μm, 71 μm, 72 μm, 73 μm, 74 μm, 75 μm, 76 μm, 77 μm, 78 μm, 79 μm, 80 μm, 81 am, 82 μm, 83 μm, 84 μm, 85 μm, 86 μm, 87 μm, 88 μm, 89 μm, 90 μm, 91 μm, 92 μm, 93 m, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, 110 μm, 111 μm, 112 μm, 113 μm, 114 μm, 115 m, 116 μm, 117 μm, 118 μm, 119 μm, or 120 μm. In certain example embodiments, the wavelength ranges from about 5 μm to about 120 μm, optionaly 5-10 μm, 10-20 μm, 20-30 m, 30-40 μm, 40-50 μm, 50-60 μm, 60-70 μm, 70-80 μm, 80-90 μm, 90-100 μm, 100-110 m, or 110-120 μm. In certain example embodiments, the wavelength is about 5 μm, 6 μm, 7 m, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 am, 33 μm, 34 μm, 35 μm, 36 μm, 37 μm, 38 μm, 39 μm, 40 μm, 41 μm, 42 μm, 43 μm, 44 am, 45 μm, 46 μm, 47 μm, 48 μm, 49 μm, 50 μm, 51 μm, 52 μm, 53 μm, 54 μm, 55 μm, 56 am, 57 μm, 58 μm, 59 μm, 60 μm, 61 μm, 62 μm, 63 μm, 64 μm, 65 μm, 66 μm, 67 μm, 68 am, 69 μm, 70 μm, 71 μm, 72 μm, 73 μm, 74 μm, 75 μm, 76 μm, 77 μm, 78 μm, 79 μm, 80 am, 81 μm, 82 μm, 83 μm, 84 μm, 85 μm, 86 μm, 87 μm, 88 μm, 89 μm, 90 μm, 91 μm, 92 am, 93 μm, 94 μm, 95 μm, 96 μm, 97 μm, 98 μm, 99 μm, 100 μm, 101 μm, 102 μm, 103 μm, 104 μm, 105 μm, 106 μm, 107 μm, 108 μm, 109 μm, 110 μm, 111 μm, 112 μm, 113 μm, 114 m, 115 μm, 116 μm, 117 μm, 118 μm, 119 μm, or 120 μm.

In certain example embodiments, the chamber comprises an outlet, an inlet, or both.

In certain example embodiments, the chamber is a well, microwell, channel, or microchannel. As described in greater detail elsewhere herein, in some embodiments, a device can contain multiple acoustofluidic contactless sample agitators and thus multiple wells, microwells, channels, or microchannels. In some embodiments, the multiple wells, microwells, channels, or microchannels can be configured for high-throughput processing or automation. Such general configurations of multiple wells, microwells, channels, or microchannels are generally known in the art and can be adapted for use with the acoustofluidic contactless sample agitators and cell separation methods described herein.

In certain example embodiments, the chamber comprises a closed top. In certain example embodiments, the chamber comprises an open top. In certain example embodiments, the volume of a liquid is about 1-1000 nL, μL, or mL. In certain example embodiments, the volume of a liquid is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 nL, L, or mL.

In certain example embodiments, the chamber is configured to fluidically coupled to one or more other chambers as described herein, a microchannel, and/or another microfluidic device. In certain example embodiments, each IDT of each pair of the two pairs of orthogonal IDTs are electrically coupled to a printed circuit board. In certain example embodiments, the chamber is any suitable three-dimensional shape. In certain example embodiments, the chamber comprises a tapered portion that begins at a position on the one or more walls and ends at the opening. In certain example embodiments, the opening is any two-dimensional shape. Exemplary two-dimensional shapes include, without limitation, a circle, an ellipse, a rectangle, a square, a triangle, any other regular polygon, any irregular two-dimensional shape.

In certain example embodiments, the chamber comprises a coated or non-coated polymeric material, glass, a metal, fused silica, a ceramic, or any combination thereof. Exemplary polymeric materials include, without limitation, polydimethylsiloxane (PDMS), polystyrene, and polycarbonate. In some embodiments, one or more of the components of the chamber can be 0-100% optically transparent or translucent, with 0% being completely (or 100%) opaque and 100% being completely transparent or translucent. In some embodiments, one or more components of the chamber is/are 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% transparent or translucent.

In certain example embodiments, at least a surface of the chamber that comes in contact with the volume of liquid is biocompatible. In other words, the surface or other component of the chamber that forms a surface of the chamber that comes in contact with the volume of liquid (or sample) is biocompatible.

In certain example embodiments, one or more components of the acoustofluidic contactless sample agitator are made from an optically transparent or optically translucent material.

In certain example embodiments, the acoustofluidic contactless sample agitator is configured to propagate surface acoustic waves in one or more wave modalities, wherein the one or more wave modalities are optionally selected from continuous wave, frequency modulation, pulse modulation, swept frequency modulation, or any combination thereof. In operation, the acoustofluidic contactless sample agitator propagates surface acoustic waves in one or more wave modalities, wherein the one or more wave modalities are optionally selected from continuous wave, frequency modulation, pulse modulation, swept frequency modulation, or any combination thereof in the chamber.

In certain example embodiments, the acoustofluidic contactless sample agitator is configured to denudate cumulus-oocyte-complexes. In operation, the acoustofluidic contactless sample agitator denudates cumulus-oocyte-complexes (COCs).

In certain example embodiments, the acoustofluidic contactless sample agitator is configured to achieve one or more patterns of acoustic streaming within the chamber. In operation. The acoustofluidic contactless sample agitator generates (or achieves) one or more patterns of acoustic streaming within the chamber. In certain example embodiments, the pattern of acoustic streaming is horizontal vortices, vertical vortices, chaotic azimuthal recirculations, poloidal flow, toroidal circulations or any combination thereof. In some embodiments, one or more patterns can occur simultaneously within a chamber (e.g., in different regions of the chamber. In some embodiments, the acoustofluidic contactless sample agitator is configured to generate a first pattern for a period of time, followed by a second pattern for a period of time. This operation of a first followed by a second can be repeated with any number of different patterns. One of ordinary skill in the art will appreciate how to program and/or configure the acoustofluidic contactless sample agitator or a device containing one or more of the acoustofluidic contactless sample agitator so as to produce one or more different patterns at the same or different times in view of the description herein.

In certain example embodiments, the acoustofluidic contactless sample agitator is configured to agitate one or more cells, particles, and/or complexes thereof present in a volume of liquid present in the chamber without imparting significant damage or result in a significant loss of one or more functions in the majority of one or more types of cells and/or particles present in the volume of liquid. In certain example embodiments, the function is viability and/or embryo development potential.

In certain example embodiments, the acoustofluidic contactless sample agitator is configured to agitate one or more complexes of cells resent in a volume of liquid present in the chamber such that one or more cells and/or cell types present in the one or more complexes are separated from the one or more complexes. In some embodiments, the cell type separated are cumulus cells, which can be separated from a COC to denudate the oocyte.

### Devices Incorporating Acoustofluidic Agitator(s)

Described in several example embodiments herein are microfluidic devices comprising one or more of the acoustofluidic contactless sample agitator of the present disclosure as described elsewhere herein. In certain example embodiments, the microfluidic device comprises a plurality acoustofluidic contactless sample agitators of the present disclosure as described elsewhere herein. In certain example embodiments, the microfluidic device is configured as a chip.

In certain example embodiments, the microfluidic device or component thereof is configured to be or is fluidically coupled with, electrically coupled with, and/or is in fluidic, electrical, optical, and/or wireless communication with one or more other devices and/or systems capable of upstream or downstream processing of one or more cells present in a volume of a liquid present in the chamber.

In certain example embodiments, the microfluidic device is configured for automatic agitation of one or more liquid samples present in one or more chambers.

Systems Incorporating the Acoustofluidic Agitator(s) and/or Devices

Described in certain example embodiments herein are sample processing systems that include (a) an acoustofluidic agitator as described elsewhere herein or a microfluidic device as in any one of as in any one of any one of the preceding paragraphs or as described elsewhere herein; and (b) one or more additional devices and/or systems capable of additional upstream or downstream processing, analyzing, manipulating, and/or storage of one or more particles, cells, and/or complexes thereof in the sample, wherein the one or more additional devices and/or systems are fluidically coupled with, electrically coupled with, optically coupled with, and/or is/are in fluidic, electrical, optical, and/or wireless communication the acoustofluidic device or microfluidic device of (a).

In certain example embodiments, the system is configured for oocyte processing and/or intracytoplasmic sperm injection. Systems and methods in addition to those described herein for oocyte processing and/or intracytoplasmic sperm injection are generally known in the art. During operation, in some embodiments, the system processes manipulates, analyzes, injects, cultures, and/or stores separated cells, such as oocytes.

In certain example embodiments, the one or more additional devices and/or systems is one or more of the following: an oocyte sorter; an oocyte immobilizing station; a spermatozoa reservoir; a spermatozoa sorter; a motile spermatozoa immobilization station; an injector; an embryo culturing chamber; one or more cell imaging devices; one or more system processors and/or controllers; one or more media reservoirs; a cell manipulation station; a spermatozoa storage chamber; an oocyte storage chamber; power source, heating and/or cooling device, or any combination thereof. The imaging devices can be cameras, microscopes and/or the like, which will be appreciated by those of ordinary skill in the art in view of the description herein. The system can include one or more controllers and/or user interfaces to communicate and/or otherwise control one or more components of the system. In some embodiments, the controllers and/or user interfaces include or are computers, mobile devices and/or the like. Exemplary systems in which the contactless acoustofluidic devices can be incorporated with include those set forth in U.S. Pat. Nos. 9,499,778; 7,186,547; 7,101,703, 7,915,044 and U.S. Pat. Pub. 2019/0308192. In certain example embodiments, system or one or more devices or systems thereof are automated or are otherwise configured for automation or automatically carrying out one or more steps of method described herein.

### METHODS OF USE

Generally, the contactless acoustofluidic agitators (and thus devices and systems which incorporate them) can be used to mix a fluid within the agitator, agitate cells, particles, or other materials that are present in a fluid within the agitator, and/or separate components of complexes that are present within a fluid within the agitator. It will be appreciated that by tuning the SAW and other components of the device that the agitation provided to the liquid and cells, particles, and/or other materials present within the liquid can be mixed, agitated, and/or separated. Thus, in this way tuning can provide different operations

Described in certain example embodiments herein are methods of separating cells from a complex of cells comprising exposing a sample comprising complexes of cells present in the chamber of an acoustofluidic agitator as in any one of any one of the preceding paragraphs or as described elsewhere herein, a microfluidic device as in any one of any one of the preceding paragraphs or as described elsewhere herein, or of a system as in any one of any one of the preceding paragraphs or as described elsewhere herein, to acoustic streaming and/or acoustic radiation force in the chamber by applying tunable orthogonal surface acoustic waves produced by the acoustofluidic agitator to the chamber, wherein the acoustic streaming produced in the chamber separates one or more cells and/or cell types from the complex of cells.

In certain example embodiments, wavelength of the surface acoustic waves is about the average diameter of the one or more cells or cell types to be separated from the complex of cells. In certain example embodiments, the acoustic streaming produced in the chamber is sufficient to denudate one or more cells from one cell in the complex of cells. In certain example embodiments, the complex of cells is a cumulus-oocyte-complex.

In certain example embodiments, the wavelength ranges from about 5 μm to about 120 μm, optionally about 5-10 μm, 10-20 μm, 20-30 μm, 30-40 μm, 40-50 μm, 50-60 μm, 60-70 μm, 70-80 μm, 80-90 μm, 90-100 μm, 100-110 μm, or 110-120 μm.

In certain example embodiments, the frequency of the surface acoustic wave ranges from about 2 MHz to about 200 MHz or more, from about 5 MHz, 10 MHz, 20 MHz, 30 MHz, 40 MHz, 50 MHz, 60 MHz, 70 MHz, 80 MHz, 90 MHz, 100 MHz, 110 MHz, 120 MHz, 130 MHz, 140 MHz, 150 MHz, 160 MHz, 170 MHz, 180 MHz, or about 190 MHz to about 200 MHz. In certain example embodiments, the frequency of the surface acoustic wave is about 5 MHz, 6 MHz, 7 MHz, 8 MHz, 9 MHz, 10 MHz, 11 MHz, 12 MHz, 13 MHz, 14 MHz, 15 MHz, 16 MHz, 17 MHz, 18 MHz, 19 MHz, 20 MHz, 21 MHz, 22 MHz, 23 MHz, 24 MHz, 25 MHz, 26 MHz, 27 MHz, 28 MHz, 29 MHz, 30 MHz, 31 MHz, 32 MHz, 33 MHz, 34 MHz, 35 MHz, 36 MHz, 37 MHz, 38 MHz, 39 MHz, 40 MHz, 41 MHz, 42 MHz, 43 MHz, 44 MHz, 45 MHz, 46 MHz, 47 MHz, 48 MHz, 49 MHz, 50 MHz, 51 MHz, 52 MHz, 53 MHz, 54 MHz, 55 MHz, 56 MHz, 57 MHz, 58 MHz, 59 MHz, 60 MHz, 61 MHz, 62 MHz, 63 MHz, 64 MHz, 65 MHz, 66 MHz, 67 MHz, 68 MHz, 69 MHz, 70 MHz, 71 MHz, 72 MHz, 73 MHz, 74 MHz, 75 MHz, 76 MHz, 77 MHz, 78 MHz, 79 MHz, 80 MHz, 81 MHz, 82 MHz, 83 MHz, 84 MHz, 85 MHz, 86 MHz, 87 MHz, 88 MHz, 89 MHz, 90 MHz, 91 MHz, 92 MHz, 93 MHz, 94 MHz, 95 MHz, 96 MHz, 97 MHz, 98 MHz, 99 MHz, 100 MHz, 101 MHz, 102 MHz, 103 MHz, 104 MHz, 105 MHz, 106 MHz, 107 MHz, 108 MHz, 109 MHz, 110 MHz, 111 MHz, 112 MHz, 113 MHz, 114 MHz, 115 MHz, 116 MHz, 117 MHz, 118 MHz, 119 MHz, or 120 MHz.

In certain example embodiments, cumulus cells are separated from an oocyte.

In certain example embodiments, the method further comprises removing a separated cell from the chamber.

In certain example embodiments, the removed cell is further processed, manipulated, analyzed, cultured, and/or stored using one or more a downstream devices and/or systems. In some embodiments, the method includes processing, manipulating, analyzing, injecting, culturing, storing, or any combination thereof the removed separated cell, optionally using one or more a downstream devices and/or systems.

In certain example embodiments, the one or more downstream devices and/or systems is/are fluidically coupled with, electrically coupled with, and/or is in fluidic, electrical, optical, and/or wireless communication the acoustofluidic device.

In certain example embodiments, one or more steps are automated. In certain example embodiments, one or more functions or operations of the agitator, device, and/or system are performed manually by a user. In certain example embodiments, one or more functions or operations of the agitator, device, and/or system are automatically performed and/or controlled by one or more computers or other computing devices.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

### Introduction

Oocyte denudation is a crucial prerequisite for unequivocal evaluation of oocyte maturity and successful intracytoplasmic sperm injection.^{1,2} Residual cumulus cells can potentially prevent the oocyte from being adequately manipulated and may represent a source of DNA contamination during trophectoderm biopsy for PCR-based preimplantation genetic testings.^{3,4} The established manual denuding method, developed decades ago, contains two steps: the enzymatic digestion and then the mechanical stripping of the cumulus using pipettes.⁵ Hyaluronidase is commonly used to break down the hyaluronic acid of the matrix encompassing the cumulus to facilitate their dispersion.⁶ Although prolonged enzymatic exposure can minimize the time and shear stress of the mechanical procedure, it has also been shown to expand the perivitelline space (PVS) and convolute the following micromanipulation.^{7,8} Despite its common usage, manual pipetting for oocyte denudation has exhibited little change over the decades and still remains inefficient, labor-intensive and fatigues the embryologist. Further, it has been shown that high shear stresses during mechanical treatment can dislocate the polar body (PB) and meiotic spindle (MS) which leads to a suboptimal ICSI outcome and poor embryo development.^{9,10} Finally, it has been found that the entire denudation procedure is highly skill dependent, time sensitive and frequently suffers from intra operator variability.^{2,11}

Unlike semen processing, female gamete processing has rarely been scrutinized by novel microfluidic technologies.¹²⁻¹⁴ Until recently the processing of cumulus-oocyte complexes (COCs) was limited to the work of Zeringue et al. who suggested a microfluidic channel that pushes the cumulus to the sides of the oocyte which makes them easier to pull off at right corners.^{15,16} More recently, Weng et al. developed a microfluidic chip that achieves multiple COC processing by physically shearing them against sharp corrugated walls. In their device, COCs are pushed against jagged side walls in a contraction section followed by an expansion section for reorientation.¹⁷ Applicant also recently showed that complete and controlled denudation is achievable in a non-contact oscillatory microfluidic channel that uses a bas-relief structure to twist the flow inside the channel. In Applicant's suggested platform the denudation extent is controlled by the fluid flow and frequency of oscillation.^{18,19} These techniques also require additional steps for preparation and delicate loading of the COCs to the devices which makes the overall process prohibitively cumbersome and time consuming. In addition, constant monitoring of oocytes due to the intrinsic 2D structure of chips and limited field of view (FOV) of the microscope is not possible. Despite some moderate success and improvements, all methods still suffer from the irreversibility of the chips. Oocytes are rare and precious cells, so any developing technology should allow their recovery at any stage in the process.²⁰

Acoustofluidics, the science of micro-manipulation of fluids and microparticles using sound waves, offers advantages over the skill demanding, labor-intensive requirements of manual pipetting by reducing the risks of high mechanical stress exposure, cell loss, and operator variability.²¹ Between the two common modalities of ultrasound generation, surface acoustic waves (SAW) are preferred over bulk acoustic waves (BAW) for micron scale applications in biology and medicine.²² These applications require high-frequency acoustic wave generation, lateral control of the acoustic wave and benefit from the ability to use transparent substrates for optical imaging systems, of which SAW is more appropriate.²³ In addition, making standing waves with SAW relaxes the limitation of using high resonant chambers and allows the use of more biocompatible, transparent but acoustically dampened materials like PDMS^{24,25} More importantly, ultrasound waves within cell scale wavelengths and energies similar to those commonly used in ultrasound imaging and diagnostic testing can be generated using planar interdigitated transducers (IDTs).^{26,27} This is of particular interest, since the impact of ultrasound on a variety of cells is being consistently examined.²⁸⁻³² Further, diagnostic ultrasound imaging has been used for more than half a century in obstetrics and gynecology and its innocuity for cells has been defined and documented in terms of thermal and mechanical indexes to prevent any potential cavitation and induced thermal damage.³³⁻⁴⁰

Using ultrasound for oocyte preparation provides a substantial evolution in the process. By gently shaking and squeezing the cells, it eliminates the potential damage that can be incurred using the current methods for COC denudation such as zona pellucida fracture, MS dislocation, PVS expansion, or oocyte activation.² The IDTs that produce the ultrasound waves are either embedded inside or circumscribed on the periphery of the microwells similar to those commonly used by embryologists for embryo development. Using grating reflectors in one direction, and a slight difference in the resonance frequency in the other, we are able to switch the acoustofluidic field direction by slightly altering the actuation frequency without using multiple signal ports. Such capability prevents any specific pattern formation and eliminates any void regions that can prevent optimal denudation. Eventually, the acoustofluidic field inside the microwell moves and tumbles the cells, continuously exposing them to shear and acoustic forces to strip off the cumulus cells. Applicant demonstrates the safety and efficacy of the method by (i) measuring the acoustic field with a laser doppler interferometer, (ii) measuring the temperature variation using a sensitive thermal camera and (iii) performing denudation and embryo development studies on mice oocytes denuded by the present method demonstrated and described in this Example compared to the conventional method.

### Methods

### Device Fabrication

Fabrication of the ultrasound denudation module consists of two major parts. First, the deposition of IDTs on the lithium niobate and fixation of the wire bonds to the PCB using potting epoxy. Second, the fabrication of microwells that sit on top of the LiNbO₃ substrate. The details of the manufacturing process of both steps are discussed in Supplemental Notes.

### Driving Stimulation Modes and Acoustic Field Intensity Calculations

Applicant calculated the intensities for the worst case scenario of maximum particle displacement without any decay throughout the microwell-substrate contact area. The acoustic intensity for SFM mode can be estimated from

$${I}_{SPTA}^{SFM} = \frac{1}{{t}_{2} - {t}_{1}} {I}_{m} {\int }_{{t}_{1}}^{{t}_{2}} {( \frac{{p}_{env} ( t )}{{p}_{m}} )}^{2} dt$$

where the integration period is chosen to be over the time of frequency sweeping (2 seconds), Iₘ=pₘ²/2pc is the maximum intensity and pₑₙᵥ, is the envelope of the swept pressure assuming a normal distribution of particle displacement as a function of frequency sweep. For the PM mode, Applicant estimated the intensity from

$${I}_{SPTA}^{SFM} = {f}_{p} {I}_{m} {\int }^{PD} {( \frac{{p}_{env} ( t )}{{p}_{m}} )}^{2} dt$$

where fₚ is the pulse frequency repetition (1 kHz) and the integral is over a single pulse (PD=500 μs). Finally, Applicant calculated the mechanical index from MI=pr.3(zSP)/(fc1/2) in which pr.3 is the peak rarefactional pressure in megapascals (MPa) derated by 0.3 dB cm-1-MHz and fc is the center frequency in megahertz (MHz).

Applicant used an Anritsu (MG3960C) RF/microwave signal generator to address the signal modulation requirements. The single port driving signal is then split by a 4 way power splitter (Mini-Circuits, ZFSC-4-1 W-S+) connected to 4 input ports of the device.

Finally, the chirp signals in SFM mode refer to sinusoidal signals whose frequency is a function of time without any amplitude modulation that can be applied further on the signals. In a predetermined time period, Applicant swept the frequency from a minimum value (f_{Amplitude/2}) to a maximum value (f_{AMplitude/2}) with a constant amplitude which can be mathematically represented by s(t)=w(t)sin(2πf₀t+πBt²/T) in which w(t) is the amplitude rectangular window of width T, B is the chirp width and f0 is the starting frequency.⁵⁵

Ultrasound Denudations Protocols with Frozen and Fresh COCs

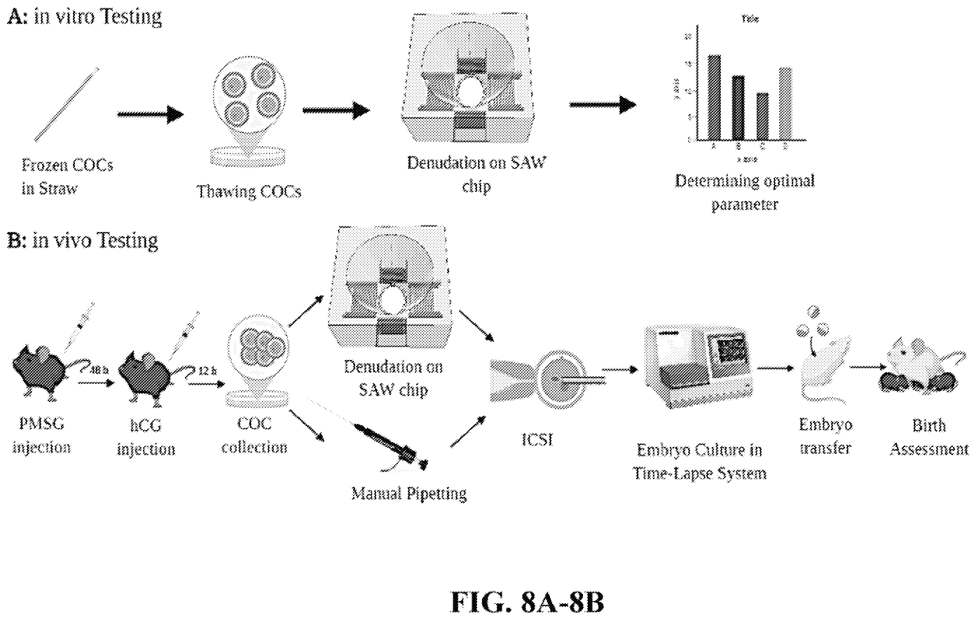

For in vitro performance evaluation and parameter optimization, Applicant tested the devices with frozen COCs. Frozen straws containing five intact COCs were thawed in a Petri dish according to provided protocols. Applicant used a 1 mm in diameter transfer pipette tip to avoid any manual denudation during the washing and transferring steps. After complete rehydration, we transferred the COCs into the microwell containing thermally equilibrated EmbroyMax M2 Medium with Phenol Red & Hyaluronidase (M2+HA) product number MR-051 from Sigma-Aldrich. Immediately after transferring the solution to the microwells, the samples were treated with ultrasound per one of the driving modalities. After the procedure, we transferred the denuded cells to a droplet on a Petri dish and acquired phase-contrast images for denudation efficiency assessment and quantification (FIG. 8A, Movie S4).

For in vivo testing, fresh COCs were retrieved from the oviducts of hyperstimulated B6D2F1 mice. The clusters of COCs were then mechanically isolated into individual COCs. The COCs were then allocated for denudation by either SAW or by conventional manual pipetting aided by hyaluronidase similar to the in vitro protocol. Piezo-ICSI was performed on denuded oocytes, and post-ICSI oocytes were cultured and monitored in a time-lapse incubator up to 96 h. Blastocysts were transferred into pseudo-pregnant 2.5 dpc CD-1 surrogates. The pregnancy, delivery, and health of pups were assessed (FIG. 8B, Movies S5 and S6).

### Image Segmentation

Analyzing the 2D images of COCs during or after the denudation procedure is a challenging and cumbersome task. This is mainly because of significant inhomogeneity in the background and foreground intensities of the images. During the denudation, the cumulus cells accumulate inside the microwell and remain in close vicinity to the oocyte. The oocytes' movements inside the microwell also have a vertical component that quickly pushes them out of focus of the camera. Despite labeling hundreds of images extracted from the acquired movies and training several convolutional neural networks (CNNs) mainly with a U-shape structure (U-Net), we were unable to produce a robust CNN model to predict denudation efficiency. Consequently, for consistent images and preventing tediousness of training new CNNs, we first acquired cell images in a droplet with a phase-contrast microscope after extracting cells from the microwell. And second, we adapted an unsupervised image segmentation method based on non-separable wavelets similar to those recently developed for gel electrophoresis image analysis.^{86,87} The details of the image segmentation method and implementation code can be found in work of Sengar et al.⁸⁷⁻⁸⁹ The image processing workflow is as follows: (1) the original images are normalized between 0 and 1. (2) The normalized images are decomposed using an undecimated non-separable quincunx wavelet to obtain the same size decompositions for better comparison. (3) A linear minimum mean square error estimation (LMMSE) based noise filtering method is applied on wavelet coefficients to capture all the singularities and noise. (4) The texture characterization and spot detection followed by an edge detection is performed in the wavelet domain to define the local textures. (5) The overlapped spots and regions of thin streaks and noise are further refined by using a morphological opening operation using a disk shape structure with a similar diameter to a single cumulus cell (10 microns). (6) The minimum energy regions are determined to remove all the edges that do not fall under this criterion as well as supersaturated spots in the image. (7) The results of previous steps are merged to render the final segmented image.

To capture all the areas associated with the cumulus complex, all the steps are repeated twice for two different scenarios of segmenting darker and brighter regions (FIG. 8A-8D panel ii and FIG. 8A-8D panel iii). Due to texture similarities of cumulus cells with zona pellucida, and oocyte cytoplasmic regions, this method also detects cytoplasmic regions as cumulus cells. As such, the final segmentation is followed by detecting the cytoplasmic region using the Hough transform algorithm. Lastly, the resulting masks of detecting darker regions, brighter regions, and oocyte cytoplasm are merged together to produce the final segmentation mask (FIG. 8A-8D panel iv).

To calculate the denudation efficiency, the untreated COC cumulus area is defined as the average of five calculated areas from 2D images of individual intact COCs. Furthermore, the residue area remaining on treated oocytes is calculated as the difference between the calculated area of treated oocytes and the average area of at least five completely denuded oocytes.

It is worth mentioning that the image analysis method is only a partial representation of the denudation efficiency. The cumulus cells are attached at all of the oocyte surfaces in 3D and a 2D image segmentation cannot capture all of them. Another weakness of this method is that it is sensitive to image quality and size inhomogeneities of cells. In the case of very small, very large, or fragmented oocytes the calculated denudation efficiency can result in non-logical values that should be either handled manually or be discarded as outliers. In addition, this image analysis method is only used for quantification, and optimization of parameters for the in vitro protocol. In the experiments with fresh COCs, the procedure is more dynamic. An experienced operator accesses the oocyte denudation progress visually by pausing the procedure. In the case of complete denudation, he stops the procedure and extracts the cells, otherwise he resumes the procedure to achieve complete denudation (Movie S5).

### Measurement and Estimation of Ultrasound Induced Thermal Dissipation

Applicant measured the acoustic induced temperature variations with an infrared camera equipped with a high sensitivity magnifying thermal lens (T300 FUR systems). Applicant used a hotplate to calibrate the emissivity of measurements for LiNbO₃, M2 and glycerol media used in the measurements. Temperature measurements were first carried out at the surface of the substrate respectively in droplets of deionized water, M2 μmedium and glycerol on top of a LiNbO3 substrate. The temperature increase on the surface of the substrate can only be attributed to power dissipation of the SAW resonators⁹⁰ while measurements in droplets give indications of both acoustic absorption and Joule heating. Further experiments also revealed that the temperature increase could be controlled by the power of the driving mode, pulse duration, PRF similar to diagnostic ultrasound, and active cooling of the substrate.

The temperature increase solely due to complete acoustic absorption can be theoretically estimated according to ΔT=Q′Δt/ρCp where Q′ is the ultrasound generated heat flux, t is the stimulation duration, Cp is the specific heat capacity of the medium and p is the medium density.^{72,80} The ultrasound generated heat also can be calculated from Q′=αP2/ρc where P is the effective pressure and α is the absorption coefficient. The temperature increase for alternating electric fields below 300 MHz can be also estimated from ∇·(km∇T)+σE2 where km is the thermal conductivity of the medium, a is the electrical conductivity of the electrolyte and E is the electric field vector.⁷⁷ Temperature rise in deionized water following theoretical estimation for CW ultrasound after 2 minutes is 1.8° C. which is higher than the experimentally measured temperature. The difference can be attributed to several factors such as inaccurate estimation of acoustic absorption, neglecting evaporation, or heat convection due to acoustic streaming. Also, temperature measurements using cell culture medium show that higher conductivity media enhance the Joule heating contribution in comparison to deionized water. Also, considering the very low electric conductivity of glycerol, the temperature increase in glycerol indicates the dominance of acoustic absorption in the medium over Joule heating.

### Animal Use and Acquisition of Gametes

For initial tests and optimization steps, we used commercially available cryopreserved metaphase II mouse (B6C3F1) oocytes (Embryotech Laboratories Inc. USA)91 with intact cumulus cells.

To obtain oocytes for the in vivo experiment, 12-week-old B6D2F1 female mice were injected intraperitoneally with 0.2 μml of pregnant mare serum gonadotropin and inhibin cocktail (CARD Hyperova, Cosmo Bio, Japan) for ovarian hyperstimulation. After 48 hours, 7.5 IU of human chorionic gonadotropin (hCG, CG10, Sigma-Aldrich, Saint Louis, MO, USA) were administered to trigger ovulation. About 16 hours post hCG trigger, the mice were euthanized by cervical dislocation. The oviducts were surgically removed and transported to the micromanipulation lab in potassium-supplemented simplex optimized medium (KSOM) (CARD KSOM, Cosmo Bio. Japan). The ampullae were punctured by a 30 gauge needle to release the COC cluster into clean KSOM droplets and allocated for manual pipetting or SAW denudation.

To obtain spermatozoa, the cauda epididymis of 10-week-old B6D2F1 μmale mice was retrieved surgically and transported in human tubal fluid medium (HTF, Irvine Scientific, Santa Ana, CA, USA). The spermatozoa were released into a clean HTF medium droplet by microdissection. The spermatozoa were incubated at 37° C., 5% CO2 and 92% humidity for 3 hours minimum prior to use for ICSI. The concentration of spermatozoa was adjusted to achieve a final concentration of 3 μmillion per mL for piezo-ICSI.

All B6D2F1 μmice were obtained from Jackson Laboratory (Bar Harbor, ME, USA). All CD-1 μmice were purchased from Charles River Laboratories (Catskill, NY, USA). All animal treatments were approved by the Institutional Animal Care and Use Committee of Weill Cornell Medicine.

### ICSI Procedure

Piezo-actuated ICSI was performed based on previous protocols with slight adjustments.⁹² A blunt injection pipette (Piezo Drill Tip ICSI, Eppendorf, Germany) was back-loaded with Fluorinert (FC-770, Sigma-Aldrich, Saint Louis, MO, USA) and attached to a micropipette holder equipped with a piezo actuator (PMM-150FU Piezo Impact Drive, Prime Tech, Japan or PiezoDrill, Burleigh, Victor, NY, USA). A mineral oil covered micromanipulation dish was prepared. Spermatozoa were loaded into the center PVP droplet and up to 10 oocytes were transferred in each surrounding M2 μmedium (CARD M2, Cosmo Bio. Japan) droplet for the ICSI procedure.

To prepare the injection pipette, residual air and a small quantity of Fluorinert were expelled into a PVP droplet. The injection pipette was then primed by suctioning PVP until smooth control of the Fluorinert-PVP meniscus was obtained. Mouse sperm heads were mechanically separated and aspirated into the injection pipette. While securing a single oocyte at 9 o'clock by the holding pipette (custom made holding pipettes, Hamilton Thorne, Beverly, MA, USA), a laser (LYKOS, Hamilton Thorne, Beverly, MA, USA) is applied to the zona pellucida to create a breach, the injection pipette was then inserted through the breach, and advanced through 80% of the oocyte forming an invagination. A weak piezo pulse was applied to breach the membrane and deposit the sperm head into the ooplasm. The pipette was retracted while aspirating gently to close the oolemma to avoid degeneration. Alternatively, the zona could be breached by applying a stronger piezo pulse without the assistance of a laser (Movie S8).

### Embryo Culture, Embryo Transfer and Pregnancy Outcomes

After ICSI, oocytes were washed thrice in KSOM before transferring into the time-lapse incubator. A single oocyte was loaded in a microwell in EmbryoSlides (EmbryoSlide, Vitrolife, Sweden) and placed in EmbryoScope (Vitrolife, Sweden). Each embryo was imaged every 10 μminutes, and its development events were annotated for up to 96 hours. Timing for embryo developmental hallmarks was compared between the control and experimental groups. Resulting blastocysts were transferred into a 2.5 dpc pseudo-pregnant CD-1 female mouse mated with vasectomized CD-1 μmale mouse. Embryo transfer was conducted either surgically or by using a non-surgical embryo transfer (NSET) device (ParaTechs, Lexington, KY, USA).⁹³ Delivered pups were weighed and monitored until weaning.

### Results

### Working Mechanism of the Tunable Ultrasonic Denudation Microwell

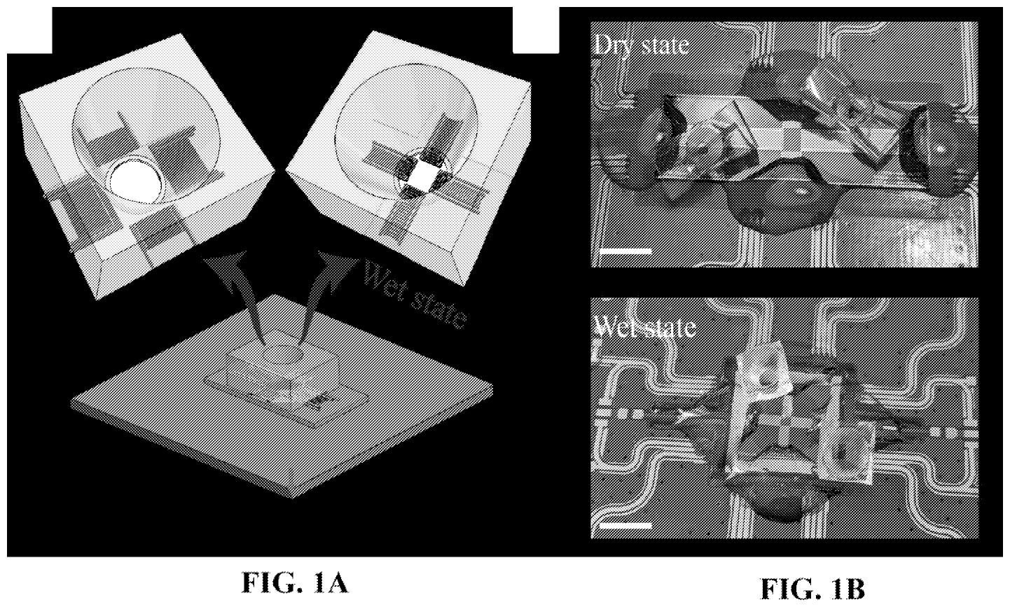

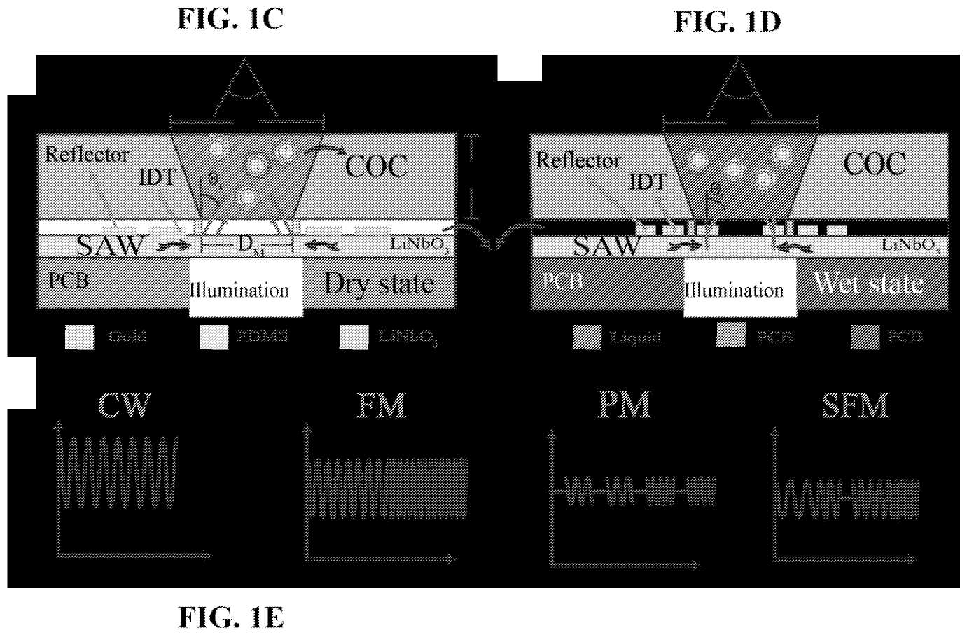

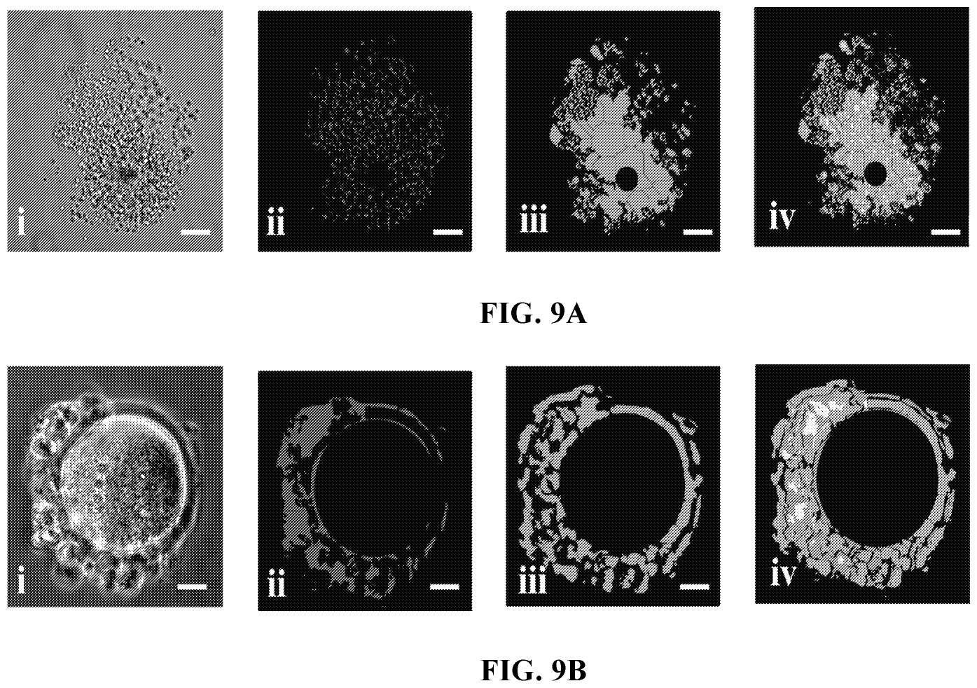

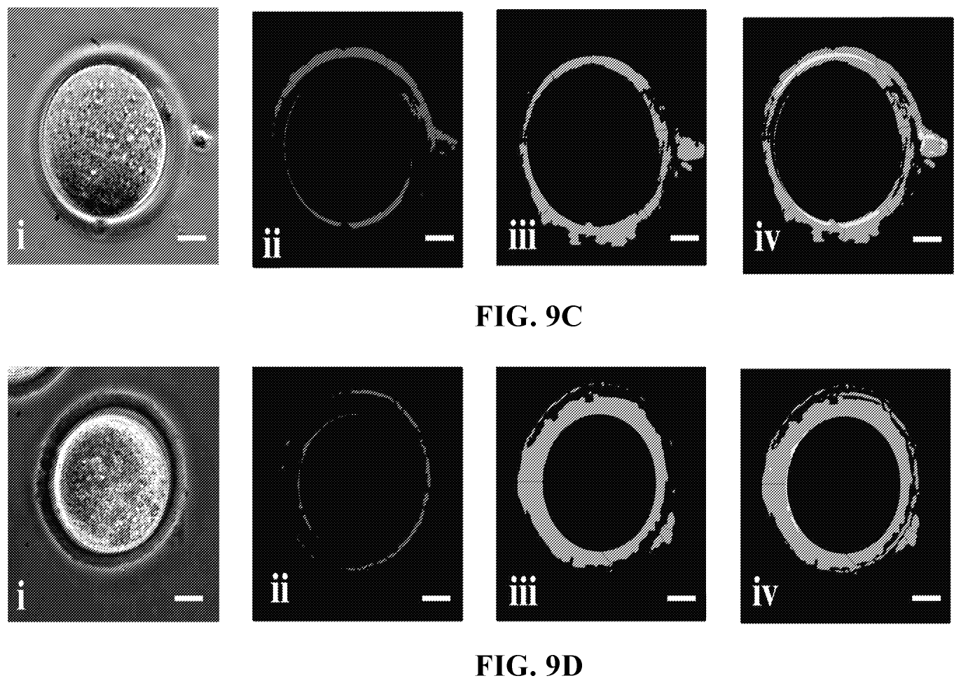

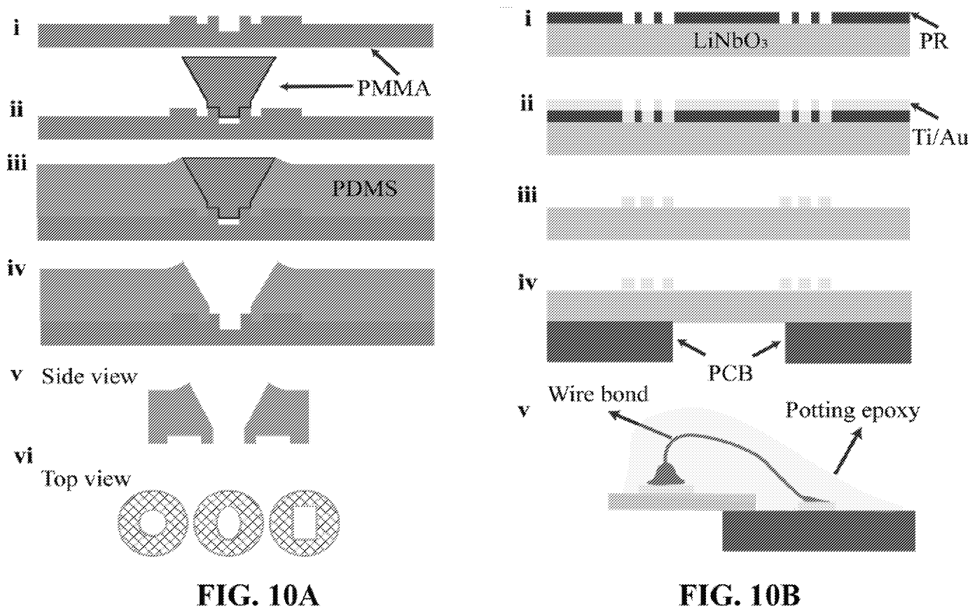

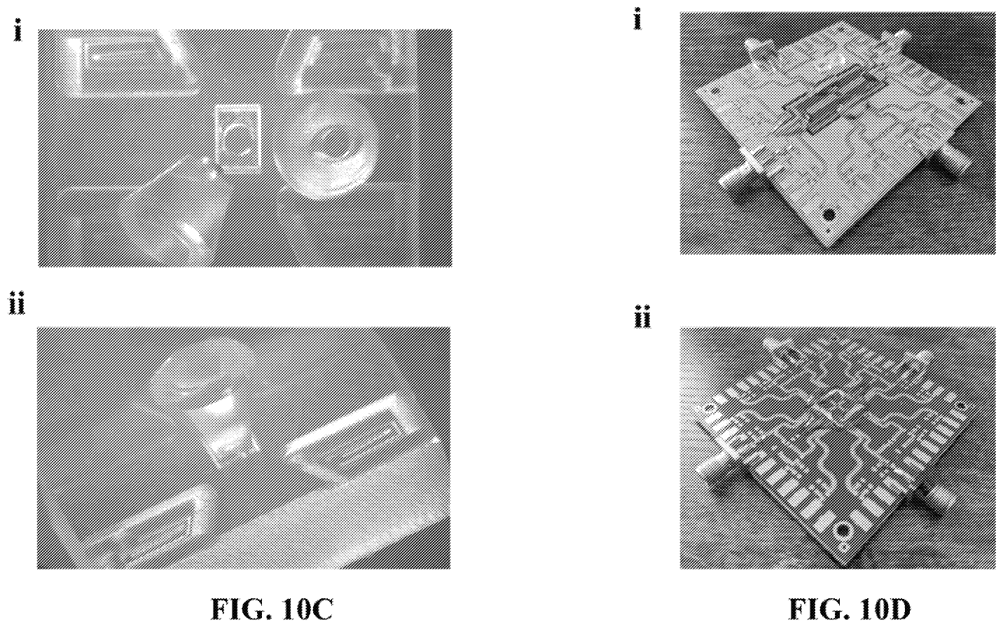

Efficient and controlled agitation inside the microwell for effective denudation of the oocytes requires complete 3D mixing of the medium inside the microwell as well as twisting the fluid flow streamlines to avoid any specific patterns and trapping regions. The devices' structure, design and working mechanism are illustrated in FIG. 1A-1F. A PDMS microwell with different open bottom geometries (FIG. 9A) is reversibly bonded to a 500 μm-thick 128° Y-cut lithium niobate (LiNbO₃) substrate between the center of two orthogonally symmetric IDTs (FIG. 1A). We choose microwell dimensions that were similar to microwells commonly used for embryo development and time lapse measurement, that had easy access for loading and unloading under a stereo microscope, and that would prevent bubble formation at the bottom of the microwell.⁴¹ It is also important to point out that it's imperative to make grooves at the bottom of the PDMS structure where the IDTs are overlaid (FIG. 10A). As shown in FIGS. 1C and 1D these grooves create an air gap between the piezoelectric substrate and the microwell structure that prevents SAW absorption by PDMS. PDMS is an effective sound absorber that can convert total SAW energy into heat if they come into contact.⁴²

To achieve thorough mixing, we established alternating 3D pressure fields inside the microwell by manipulation of two orthogonal surface acoustic waves (SAW). Due to the acoustic impedance mismatch between air and water, the SAW propagation is disturbed upon entering the liquid filled microwell, resulting in an exponential decay along the solid liquid interface (leaky Rayleigh SAW) and 3D longitudinal sound wave radiation into the liquid.⁴³ As such, acoustic streaming in the form of laminar jet flows emanates from the periphery of the microwell driven by the momentum flux transfer of the beam to the fluid as the beam attenuates (propagates) through the liquid medium.⁴⁴ These 3D waves launch into the liquid at a Rayleigh angle of 22° according to Snell's law θ=sin−1 (Cl/Cs) where Cl and Cs are the sound velocity in the liquid and solid substrate, respectively.^{27,45} In bioengineering applications, it is this efficient energy transfer characteristic that gives rise to acoustic radiation forces and acoustic streaming (Eckart streaming) necessary for particle or cell manipulation inside fluid filled cavities.^{26,46-49} In comparison to other fluid streaming methods, acoustic streaming control, such as switching on and off, can occur instantly in comparison to hydrodynamic timescales. Further, acoustic streaming is contactless and less sensitive to fluid viscosity, making it a versatile tool for creating an exciting range of fluid motions. We choose ˜80 MHz and ˜200 MHz frequencies with wavelengths (λs=Cs/f)of˜50 μm and ˜20 μm, respectively, on the substrate (CLiNbO₃=3980 [m s−1])⁵⁰ as working frequencies (Table 1).

TABLE 1
Corresponding resonant frequencies, actual wavelengths, and damping
(attenuation) lengths (xs, xf) listed for each propagation direction.

| | Frequency, | Wavelength on | Wavelength in | Damping length on | Damping length in |
|---|---|---|---|---|---|
| | actual f, MHz | SAW [μm] | water [μm] | SAW, xₛ [μm] | water x_{f} [mm] |
| x direction | 79.29 | 50.3090 | 18.8927 | 279.6796 | 10.1375 |
| y direction | 81.19 | 49.1317 | 18.4505 | 273.1346 | 9.6686 |
| x direction | 199.14 | 20.0311 | 7.5223 | 111.3578 | 1.6071 |
| y direction | 202.21 | 19.7270 | 7.4081 | 109.6672 | 1.5587 |

saw = 3989 m/s and F = 1498 m/s²

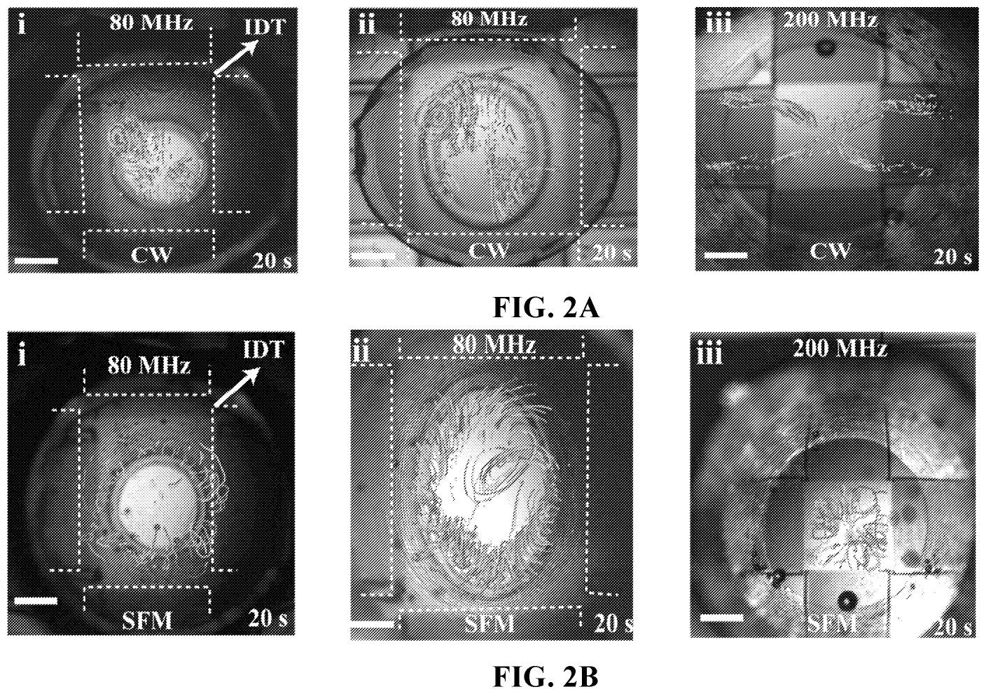

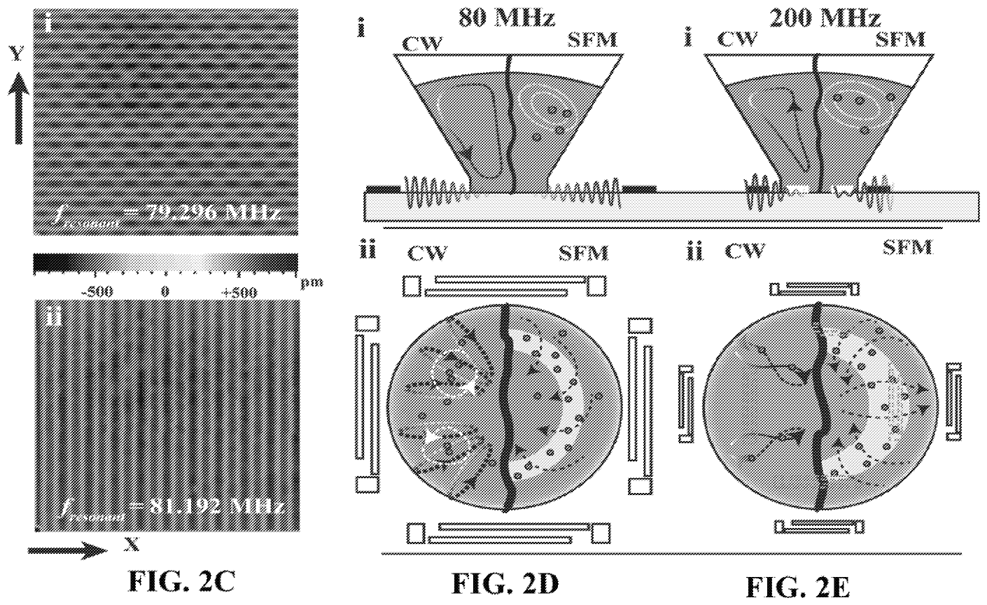

Upon attenuation into the liquid medium these frequencies remain constant but due to the slower speed of sound propagation through water (C_{water}=1498 [m s⁻¹])⁵⁰ the wavelengths are reduced to about 7.5 μm and 15 μm, respectively. These wavelengths are in the range of a single or small cluster of 2-3 cumulus cells (acting as Mie particles), resulting in higher force experience in a standing wave configuration.^{51,52} Further details on the acoustic induced fluid flow, attenuation lengths, acoustic radiation force and drag force on particles are provided in the “SAW induced acoustofluidic fields” section, FIGS. 2A-2E and Table 1.

The acoustically active areas encompassed by the IDT pairs on the substrate are either 1×1 mm² for a 200 MHz device or 2.5×2.5 mm² for an 80 MHz device. In the case of the larger active area and working frequency of 80 MHz, the IDT pairs reside outside of the fluid vessel and are excited in a dry state, while in the case of a smaller active area and working frequency of 200 MHz, a portion of IDTs resides inside the fluid vessel and is excited inside the fluid in a wet state (FIGS. 1C and 1D). The latter usage of IDTs inside the microwell also requires an extra passivation layer for IDT protection and the prevention of inadvertent cell damage due to exposure to charge carriers via dielectrophoresis. No suppression of the generated SAW was observed even at the higher conductivity level of the saline solution in the absence of the passivation layer.⁵³ Although the dry state IDTs have fewer fabrication complications and are easier for impedance matching, SAW attenuation is more significant due to its contact with the PDMS microwell and viscous heating of the walls.⁵⁴ The IDTs in the x direction of LiNbO₃ consist of five interleaving fingers with an aperture of 50λ which are backed by a reflector grating of 300 shorted electrodes, while the orthogonal IDTs in the y direction only consist of 20 pairs of interleaving electrodes with slightly different width and spacing from the x direction IDTs without any reflector gratings. The slight difference in the IDT spacing in the x and y directions, and the narrower resonance frequency bandwidth in the x direction allow us to instantaneously switch the direction of standing waves in the microwell cavity such that cells experience changes in the flow and agitation patterns (FIG. 2C, Movies S1 and S2). The acoustic streaming in the dry state device is created by the attenuation of acoustic energy from the sound beam propagating inside the liquid in the microwell,44 while in the wet state device the waves are locally coupled with the fluid that covers the stimulating transducers (FIGS. 1C and 1D).

The driving modalities employed in this study are shown in FIG. 1E panels(i-iv). In the continuous wave (CW) mode, the actuation frequency is set at the resonant frequency of the IDTs in the x direction while in the frequency modulation mode (FM) the actuation frequency is switched between x and y direction resonant frequencies at predetermined intervals of 2 seconds. In the pulse modulation mode (PM) the resonant frequency of the IDTs in the x and y directions is pulsed at 1 kHz with 50% duty cycle for 2 second intervals. Lastly, the swept FM mode (SFM) consists of two chirp type excitons for x and y direction resonant frequencies. Applicant swept the frequency from fⁱresonant−Δⁱf to fⁱresonant+Δⁱfⁱ(i=x, y) for a predetermined time interval of 2 seconds, alternating between resonant frequencies in each direction. The sweeping bandwidths are determined from FWHM measured in each direction independently.⁵⁵ Applicant developed the last actuation scheme due to two reasons: first the quality factor of the standing wave is impacted by medium loading which causes a resonant frequency shift; and second, the dependency of resonant frequency on temperature and its unwanted shifts due to acousto-thermal phenomena that resulted in inferior device performance efficiency⁵⁶⁻⁵⁸ (FIG. 10A-10D).

### SAW Induced Acoustofluidic Fields

Upon applying a high amplitude 80 MHz CW excitation signal in the dry state devices (FIG. 2A (panels i and ii)) streamlines with four vortical structures appeared, despite the contact geometry of the microwell, these concentrated the particles into a few vertical rings which are depicted schematically in FIG. 2D (i and ii) and in Movie S3(a). A small population of particles was also trapped on the surface vortices that formed on the fluid surface (FIG. 10A-10D). When Applicant applied a high amplitude 200 MHz continuous signal to wet mode devices, stronger fluid jets were generated inside the fluid that concentrated all the particles into tightly formed vertical loops (FIG. 2A (panel iii), Movie S3(c)). The flow behavior and resulting patterns can be described in terms of damping length on the substrate xₛ=0.45λₛ(ρₛcₛ)/(ρⱼc_{f})⁵⁹ and damping length in the water x_{f}=3ρₛcₛ³/16π²ηf² (ref. 60) (Table 1). For both dry and wet devices, xₛ is smaller than the microwell contact diameter (DM); this ensures that the generation of acoustic streaming rather than particle accumulation on the lines associated with standing waves forms inside the fluid in the case where xₛ>DM. For the lower frequency dry state device, x_{f} (>10 mm) is larger than the fluid height in the microwell. As such, the acoustofluidic field has minimal decay over the fluid height and reflects back at the air-liquid interface. The recirculated flows launched from each IDT interact with each other as well as the microwell boundaries, to create vertical vortices that stretch from the center of the acoustic cavity to two flanks of the IDTs in the x direction (FIG. 2D (panel ii) and FIG. 2E (panel ii)). For the high frequency wet state device, the x_{f} is smaller than the fluid height inside the microwell. Hence, the acoustofluidic field dissipates before reflection at the air-liquid interface creating a gradient closer to the microwell bottom, resulting in lower particle velocities near the fluid surface (Movie S3(c) and(d)). In both devices, the drag force, F_{drag}=3πηdₚv≈10⁻⁸−10⁻⁹ N, where v is the acoustic streaming velocity⁶¹ exerted on the suspended 90-100 μm polystyrene particles, is comparable to the acoustic radiation force stemming from attenuation of sound waves F_{rad=πd}ₚ²(I/4c)Yₚ≈10⁻⁷−10⁻⁸ N, where I is the acoustic intensity and Yₚ is the acoustic radiation force function of the polystyrene sphere.⁶²⁻⁶⁵ Without being bound by theory, Applicant suspects that the particle's motion in vertical loops consists of an outward push of acoustic streaming and inward pull of acoustic radiation force.^{60,61} Applying an SFM modulated signal breaks these fixed symmetrical patterns and results in chaotic azimuthal recirculations as shown in FIG. 2B (panels i-iii). Interestingly, applying the SFM modulated signal to a microwell with elliptical bottom geometry creates a poloidal flow with toroidal circulations as shown in FIG. 2B (panels ii), Movie S3(b).

### Considerations for SAW Intensity and Dissipated Energy

### Acoustic Intensity

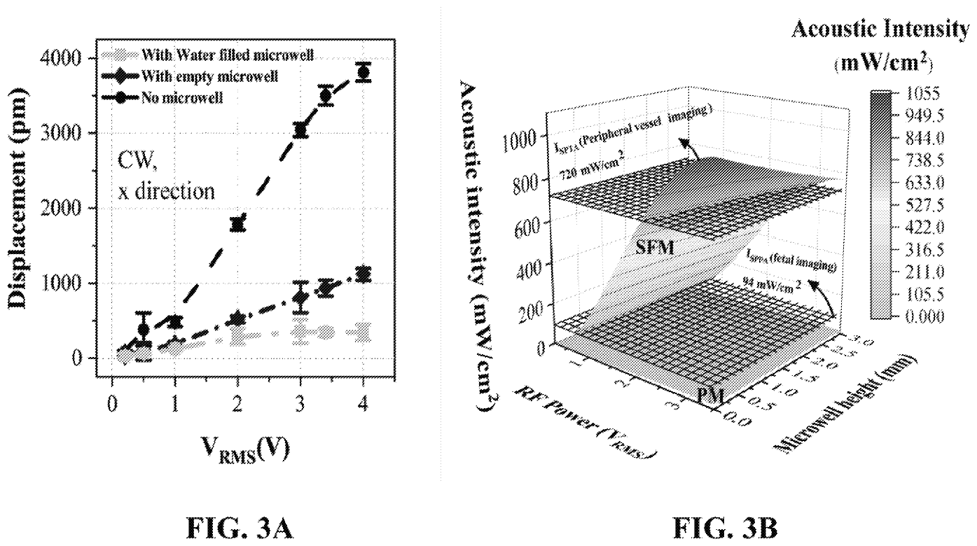

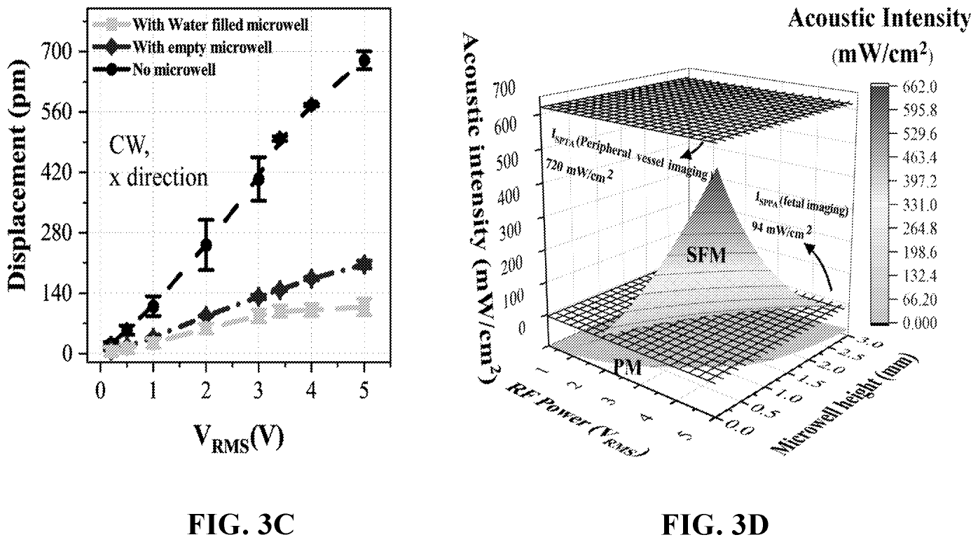

Neither the diagnostic nor the therapeutic uses of ultrasound are unconditionally safe for use in modern assisted reproductive medicine (ART).³⁹ Although the safety thresholds and operation conditions have been consistently examined, the exact effect of ultrasound output parameters such as frequency, intensity, pulse duration and frequency that are used to drive the safety indices, (i.e. mechanical index (MI), spatial-peak temporal-average intensity (Ispta), thermal index (TI)) on biological matter is not completely understood.^{37,66,67} Nonetheless, the mandated FDA thresholds in the form of safety indices (Ispta, MI, TI), which convey the probability of ultrasound induced biological effects, are well accepted by the medical community.³⁶ The majority of clinical ultrasound applications use intensities between 0.03 and 1.0 W cm⁻², and it has been shown that intensities over 3000 μmW cm⁻² can have severe biological ramifications and may lead to cell apoptosis.38,68,69 Further, MI<0.7 for bubble-perfused tissue samples and MI<1.9 for samples without bubbles are the advised upper limitation for safe ultrasound operation. The drive frequency, amplitude, modulation and exposure duration are the major parameters contributing to the amount of energy impinging on the biological samples.^{70,71} Hence, development of ultrasound denudation protocols that ensure lower intensity levels by at least one order of magnitude is necessary to achieve a therapeutic outcome while remaining below the toxicity threshold.^{37,72} The average acoustic intensity can be estimated from [symbol] I [symbol] =∫₀^{T}p(t)v(t)d(t) where p(t) is the sound pressure and v(t) is the particle displacement. The transmitted pressure is a function of transmitted particle displacement v as |pt|=ρcωv, where ρ, c, ω are the density, sound speed and angular frequency, respectively. In the experiments, Applicant measured the particle displacements of the LiNbO₃ substrate in three conditions: unloaded, loaded with an empty microwell, and loaded water filled microwell as shown in FIGS. 3A and 3C. The intensity decay inside the medium can be further estimated as I=I0e−αx where α=xf−1 is the absorption coefficient of ultrasound in water assuming close compositional similarity of media with water (see Methods). Both dry and wet state devices can be stimulated within the therapeutic window (<3000 μmW cm⁻²) which is close to the recommended FDA intensity levels for peripheral vessels and fetal ultrasound imaging (FIGS. 3B and 3D).^{36,39,73} The worst-case scenario analysis of the MI, considering the largest particle displacement and SFM mode of actuation, also revealed that the MI values of the dry state and wet state devices are 0.0537 and 0.0849 (see Methods) which are well within the recommended region (MI<0.7). It is important, however, to note that the direct experimental measurement of acoustic intensities at these wavelengths is challenging, and requires specialized hydrophones. Furthermore, the biological effects of a SAW on cell safety and viability in chip scale levels may differ from the conventional culture dishes and are hard to model.³² Thus, Applicant experimentally investigated the cell viability and development potential of mice embryos and pups procured with our device and compared them to the control group of conventional artificial insemination methods.

### Ultrasound Induced Thermal Effects

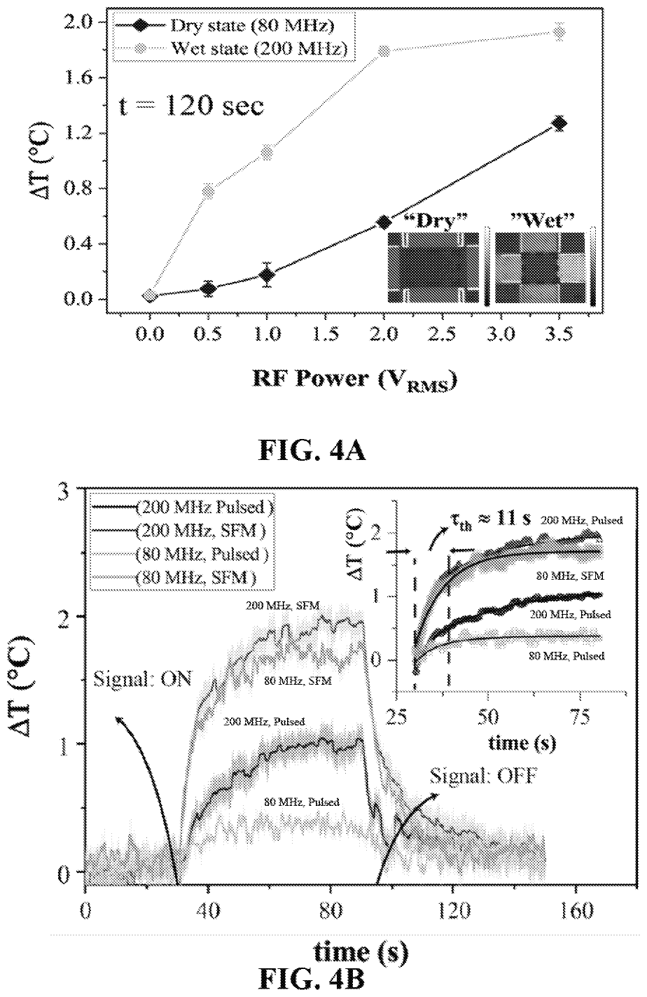

The rise in the temperature has always been a concern while using ultrasonics in screening of developing embryo/fetus due to the teratogenic effects of ultrasound induced hyperthermia.^{74,75} As such, Applicant investigated the acousto-thermal effect (energy conversion from acoustic to thermal) of our device for the denudation procedure. The viscous dissipation of acoustic energy is the main source of generation of heat inside the liquid and the PDMS microwell.^{42,76} Various insertion losses associated with the generation of SAW, and Joule heating are the two other sources that may contribute to the heat generation while performing a denudation procedure.^{77,78} It is also important to note that LiNbO₃ is hysteresis free and in generating SAW it does not produce heat.⁷⁹ The Joule heating here is related to the SAW accompanying electric field on the piezo substrate and solution electrical conductivity.⁷⁷ Applicant measured surface temperature variation of a bare LiNbO₃ substrate with a CW signal at different driving powers after reaching a steady value (120 s) using a high sensitivity infrared camera. Results shown in FIG. 4A are the spatial average temperatures in the acoustic cavity (shown in the figure inset) for both the dry and wet state devices as a function of the driving signal power averaged over a 10 second period after 120 seconds of stimulation. In the worst case scenario after applying a FM driving mode with a power amplitude of P=3.5 Vᵣₘₛ (corresponding to a maximum displacement of ≃400 pm in water) to a microwell filled with glycerol (a medium with a high acoustic absorption coefficient and low electric conductivity of 0.064 μScm⁻¹), the temperature increased by about 2.3° C. for the dry state device, and 1.8° C. for the wet state device, after 2 min with a time constant (τ)of 11 seconds. The time constant is the elapsed time the device requires to reach ˜67% of the final steady state temperature indicating the dynamic behavior of the denudation device to a step input function. In these experiments, electrical stimulation started after 30 seconds of thermal camera recording initiation and switched off after one minute, followed by another minute of recording. After switching off the power, the temperature returned to initial values with approximately the same time constant. However, with PM driving signals (1 kHz, 50% duty cycle, 1 second intervals) the temperature only increased by 0.4° C. for the dry state device and 0.2° C. for the wet state device. The dynamic temperature variation of M2 medium inside the microwells of both devices was also measured and the results are shown in FIG. 4B representing the denudation protocols using both devices. Further experiments also indicate that the coexisting electric field plays an important role in acoustic induced heating through the Joule heating mechanism which can be alleviated by active cooling of the substrates (see Methods).

Conceivably, temperature can modulate enzyme activity through reversible perturbation of protein structures among other various mechanisms.⁶ Considering that hyaluronidase (HA) is catalyzing a chemical reaction that is happening on the surface of the cumulus cells, the small temperature increase on the cell surfaces, due to the higher absorption coefficient in comparison to the medium (Z≃1.55×106 kg m⁻² s⁻¹ in soft biological tissue),⁸⁰ can act as a thermal driving force assuming Michaelis-Menten kinetics under non-isothermal conditions while neglecting the effect of temperature on reaction kinetic coefficients. This phenomenon can be further coupled to the chemical driving force, reinforcing the reaction rate.⁸¹ In summary, the temperature increase for most of the experiments used for the denudation of fresh COCs remains lower than 2° C., well below the recommended safety temperature increase limit (thermal index of 6) in ultrasonic diagnostics.⁷⁸

### Decoupling Electrical Effects in the Denudation Procedure

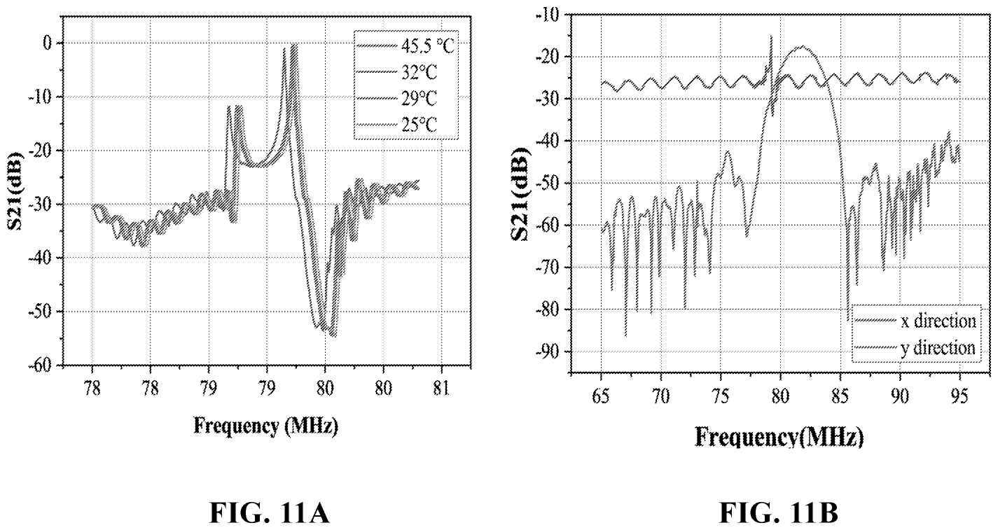

The propagating surface acoustic waves created by the reverse piezoelectric effect (conversion of electrical energy to mechanical displacement) create dynamic electric fields in the free area between the IDT transducers by the piezoelectric effect (conversion of mechanical energy to electrical energy). Qualitatively, surface acoustic displacements cause surface charges through the piezoelectric effect; the additional attraction between the surface charges causes additional strain, further stiffening the substrate and increasing the surface acoustic wave velocity.⁸² Hence, the coexisting electric field with the surface acoustic wave can induce electrical stimulation and dielectrophoresis effects.⁸³ It has also been shown that high frequency sinusoidal electrical stimulation (above 100 kHz) can alter the dynamics in excitable cells, block ion channels and lead to electrical quiescence.⁸⁴ Furthermore, it has been indicated in the literature that high SAW undulatory displacements (≅10 nm, 10 MHz and power ≳10 Vᵣₘₛ) can locally accumulate charges sufficient to create a half wavelength “nano-electrochemical” cell that may split water and create free radicals.⁸⁵ As such, it is important to understand and identify the sources of electrical fields that may affect the performance of the device. Two main sources of unwanted electrical fields in our experimental design can be either through parasitic electric signal coupling from wire bonds, or electrical fields associated with SAW. Depending on the conductivity of the medium on top of the substrate, mirror charges can form which potentially decrease the strain and velocity of the surface wave. The conductivity of the culture medium is 19 μScm⁻¹ (856 conductometer module, Metrohm, Herisau, Switzerland) which significantly lowers the electric field vector inside the medium and thus lowers any dielectrophoresis forces. In addition, the electric field exponentially decreases with distance from the bottom of the substrates. As such it is safe to assume that cells being agitated a small distance from the substrate are not exposed to any SAW mediated electric fields. For confirming this hypothesis, we used 1 μm polystyrene particles dispersed both in deionized water and M2 solution and exposed them to CW mode acoustic agitation at 24 dBm. The similarly assembled clusters of particles in both deionized water (low electrical conductivity) and M2 culture (higher electrical conductivity) medium indicate that the acoustic field has the main mechanism of particle entrapment. For further confirmation, Applicant drove the 80 MHz acoustic module at off resonant frequencies of 30 MHz and 150 MHz with a higher RF power of 30 dBm (FIG. 11A-11B). Under these conditions of weak electromechanical coupling and imperceptible SAW coupling in the fluid, Applicant did not observe any microbead assembly. This evidence also confirms the absence of parasitic electric fields (wire bonding, PCB board, etc.). Finally, for electromechanical coupling conservation in the wet state device with the immersed IDTs inside the microwell, the substrate surfaces were shielded with either a self-assembled monolayer (SAM) film of trichlorosilane53 or a thin layer of silicon dioxide.

### Denudation Efficiency

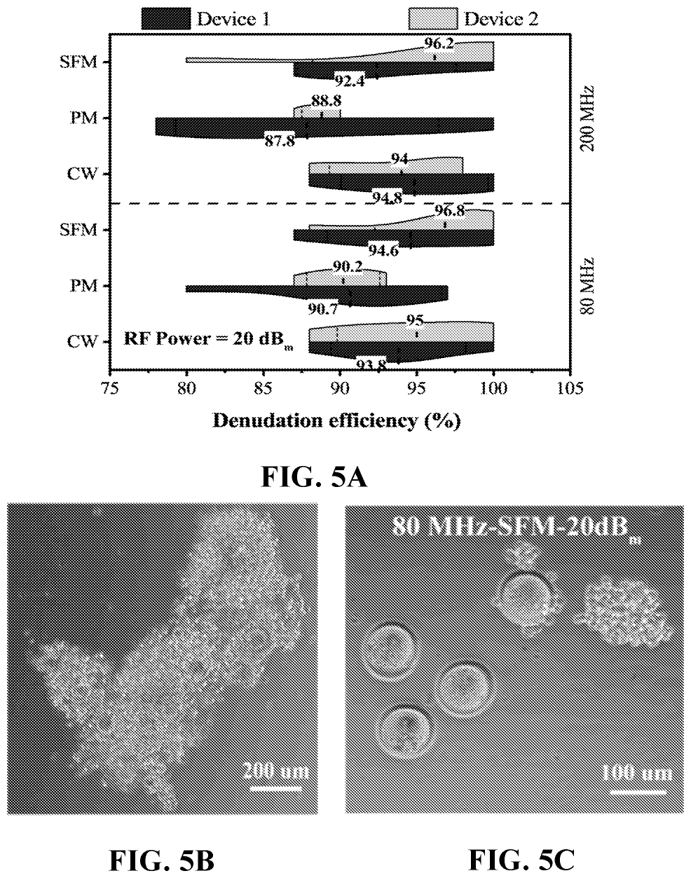

Applicant investigated the performance of each device and examined device-to-device variability by following the frozen COC denudation protocol in two different devices for both the wet and the dry modules. Applicant optimized three parameters: driving modality, power, and exposure time for achieving a complete denudation in the safest yet most efficient way. First, to compare the three driving conditions and device performance, Applicant chose, based on intensity measurements, 20 dBm (2.236 Vᵣₘₛ) as the working RF power and a 90 second exposure time. It is important to note that using different actuation modalities, even using the same driving power, results in different intensities. For the dry state module, the CW, PM, and SFM modes of actuation yielded a mean denudation efficiency of 93.8%, 90.7% and 94.6%, respectively, for device-1 compared to a mean denudation efficiency of 95%, 90.2% and 96.8%, respectively, for device-2 (FIG. 5A). Denudation efficiency is defined as the ratio of (A_{untreated}−A_{treated})/A_{untreated}, where A is the area of cumulus cells, determined through image processing of the phase contrast images taken of the oocytes before and after the procedure (FIG. 9A-9B). It is worth noting that the yield of the device, as in recovery of whole oocytes, can be considered 100% excluding oocyte loss due to human error during transfer steps. Similarly, for the wet state module, the CW, PM and SFM mode of actuation resulted in 94.8%,⁸⁷0.8%, and 92.4% denudation efficiency for device-1 which are comparable to 94%, 88.8%, 96.2% denudation efficiency of device-2. Two way ANOVA analysis indicates that at level 0.05 the population means of frequency factor are not significantly different (p=0.36) but at level 0.05, the population means of actuation modes (CW, PM, SFM) are significantly different (p=8.9×10⁻⁴). FIGS. 5B and 5C show a representative result of the SFM mode denudation procedure using the aforementioned parameters. Applicant chose the SFM mode of actuation as the most effective mode of actuation and optimized the power and exposure time consequently.

### Effect of Actuation Power and Exposure Time

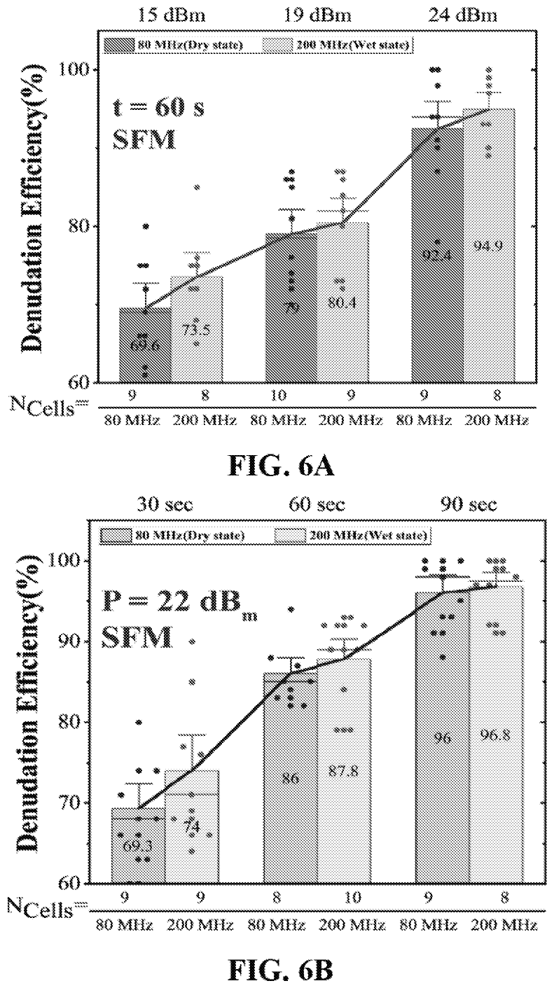

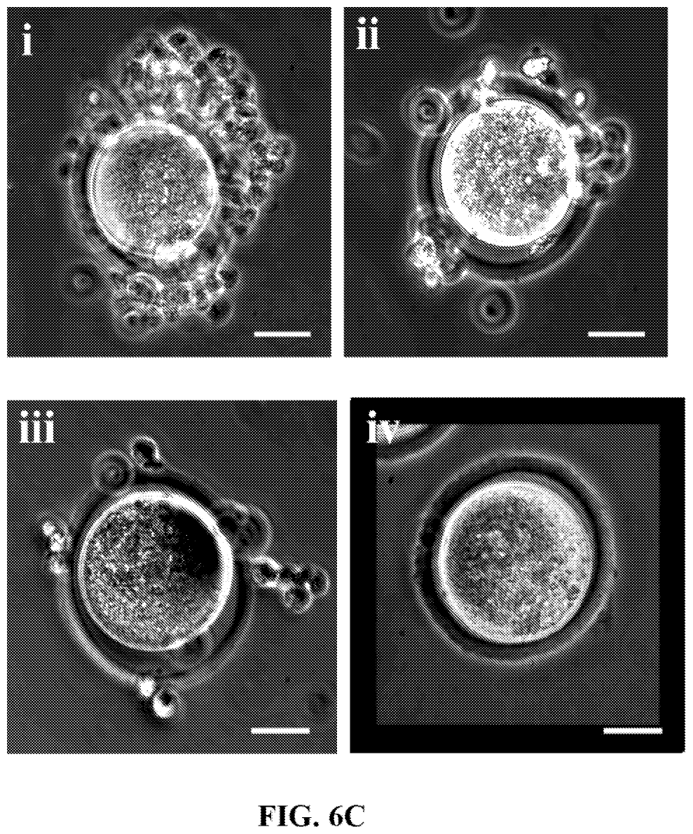

The driving stimulation power (amplitude) and exposure time are the two main parameters that determine the amount of energy inserted into a sample. For comparing the effect of power on the denudation efficiency, we used driving stimulation powers of 15 dBm (1.257 Vᵣₘₛ), 19 dBm (1.993 Vᵣₘₛ), and 24 dBm (3.544 Vᵣₘₛ) for a 60 s exposure time. As expected the denudation efficiency increases with increasing driving power for both devices as shown in FIG. 6A. This is due to the larger shear stress that cells experience due to the higher acoustofluidic flow at higher power. From the particle velocity measurements, the drag and acoustofluidic forces are approximately three times higher when using the highest driving stimulation in comparison to the lowest level. This is expected as the total force acting on the body of the fluid is assumed to have a linear relationship with the acoustic power (see Supplemental notes). Furthermore, the mean denudation efficiency also indicates a linear relationship with applied stimulation power. Applicant can infer then that the denudation efficiency is a function of the drag force intensity which has a linear relationship with the acoustic streaming magnitude. Consequently, Applicant chose 22 dBm (2.815 Vᵣₘₛ) as the driving power and investigated the effects of exposure time. As shown in FIG. 6B the denudation efficiency also increases with exposure time because of the extended chance of uniform exposure of all sides of the oocyte to shear stresses. From exposure time experiments Applicant realized that as the time of exposure increases, oocytes are more uniformly denuded in comparison to applying higher driving forces in shorter time periods. Representative images of partially denuded and completely denuded COCs using the ultrasound devices are shown in FIG. 6C (panel i-iv). Pearson correlation tests reveal a statistically significant correlation between the power level and denudation efficiency for both dry and wet state modules (P value=0.011 and P value=0.043, respectively) as well as the denudation period and denudation efficiency for both dry and wet state modules (P value=0.045 and P value=0.038, respectively). In all cases the correlation coefficients are r>95%. However, a two-tail Student test shows no significant difference between wet and dry state modules for the denudation procedure given either individual operating conditions or collective conditions (p-value=0.79 and p-value=0.82 for power and denudation period, respectively). As mentioned previously, the biological effects of SAW on cells in miniaturized experiments are very hard to model and predict. As such, Applicant carried out artificial insemination experiments to directly investigate the impact of ultrasound on the development potential of mice oocytes.

### Fertilization and Development Potential of Ultrasonically Denuded Oocytes

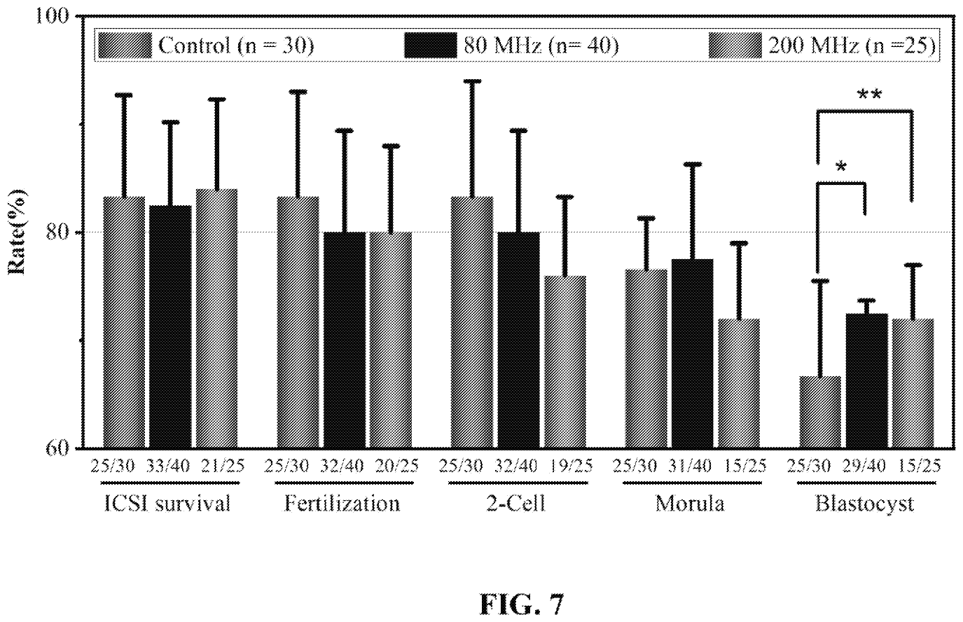

To test the efficiency and safety of the device, 40 oocytes denuded by 80 MHz SAWs, 25 oocytes denuded by 200 MHz SAWs, and 30 oocytes denuded by the manual protocol (MP) serving as the control were inseminated by piezo-actuated ICSI. The device significantly reduced the labor of the process, and the denudation quality remained the same without any oocyte loss. After piezo-actuated ICSI, the 80 MHz, 200 MHz, and MP groups yielded comparable survival rates of 82.5%, 84.0% and 83.3% (P=0.96), respectively. Fertilization rates were also comparable between the three groups at 80.0%, 80.0%, and 83.3% (P=0.88), respectively, as well as blastulation rates of 72.5% vs. 72.0% vs. 66.7% (P=0.69), respectively (FIG. 7). After reviewing embryo development in the time-lapse imaging system, all three groups had similar morphokinetics with no incidence of extensive embryo fragmentation or abnormal cleavage (Table 2). After transferring the resulting blastocysts into recipient mice, 9 live births were achieved from the 80 MHz group, while 5 were achieved from the 200 MHz group (Table 3). All pups were weaned to date without compromised development and are fertile (Movie S7).

TABLE 2
Comparison of timing of each embryo developmental stage

| Timing (h) | | tPB2 | tPNa | tPNf | t2 | t3 | t4 | t5 |
|---|---|---|---|---|---|---|---|---|
| Control | | 1.71 ± 0.59 | 5.04 ± 1.58 | 14.16 ± 0.99 | 16.19 ± 1.24 | 36.78 ± 2.72 | 38.18 ± 3.17 | 49.49 ± 3.88 |
| 80 | Mhz | 1.97 ± 0.67 | 4.36 ± 0.74 | 14.47 ± 1.07 | 16.45 ± 1.10 | 35.93 ± 1.85 | 36.91 ± 1.98 | 47.65 ± 3.11 |
| 200 | Mhz | 1.90 ± 0.65 | 4.35 ± 0.56 | 14.26 ± 1.09 | 16.17 ± 1.30 | 36.07 ± 2.50 | 37.30 ± 2.82 | 47.41 ± 3.71 |
| Timing (h) | | t6 | t7 | t8 | tM | tSB | tB | tHB |
| Control | | 51.49 ± 3.88 | 53.78 ± 6.75 | 58.82 ± 7.79 | 63.94 ± 6.13 | 75.26 ± 3.22 | 78.61 ± 2.66 | 88.13 ± 1.90 |
| 80 | Mhz | 47.65 ± 3.11 | 48.53 ± 3.15 | 49.43 ± 3.24 | 57.45 ± 3.90 | 73.54 ± 1.46 | 77.35 ± 2.42 | 90.38 ± 3.85 |
| 200 | Mhz | 47.98 ± 3.70 | 48.66 ± 3.62 | 57.78 | 51.49 ± 4.98 | 77.03 ± 2.50 | 81.28 ± 4.21 | 88.91 ± 1.62 |

TABLE 3
Embryo transfer and pregnancy outcomes

| | | | | Number of | | | | |
|---|---|---|---|---|---|---|---|---|
| | | Total Blastocysts | Number of Total | pregnant | | | Average Birth | |
| | | Transferred | Recipients | Recipients | Born | Alive | weight (g) | Weaned |
| Control | | 20 | 2 | 2 | 8 | 8 | 1.58 ± 0.41 | 8 |
| 80 | MHz | 29 | 3 | 3 | 10 | 9 | 1.62 ± 0.36 | 9 |
| 200 | MHz | 15 | 2 | 2 | 5 | 5 | 1.60 ± 0.11 | 5 |

### SUMMARY

In this Example, Applicant at least demonstrates development of a contactless 3D cell agitation platform for oocyte denudation by reshaping 2D SAW wave fields inside a biocompatible microwell, by modulating excitation signals. Applicant investigated the flow patterns, potential denudation mechanisms and safety of our developed devices. The arrangement of IDTs, small differences in IDT spacing, and reflector grating use in one direction provided Applicant with an efficient method to switch the flow inside the microwell by modulating only the excitation signals. The signal modulation is also used for controlling and keeping the acoustic intensity within the recommended FDA limits for peripheral vessels and obstetrical and gynecological imaging.

With the current experimental design, Applicant are able to carry out up to 30 oocyte denudation in less than three minutes which significantly improved the procedure efficiency and reproducibility while minimizing enzymatic treatment and reducing the out-of-incubator time. The safety of this device is validated by normal embryonic morphokinetics and live births from the mouse oocytes denuded in this study.

The simple design and straightforward setup of Applicant's device in combination with low power density requirements indicate that our technique is an efficient and safe method for preparation of oocytes for ICSI procedures. Applicant's technique also has the potential to be modified and integrated with a small RF supply with simple electronics. This will allow it to function as a portable, inexpensive, and automated device that yields reproducible results and expands the reach of ICSI procedures in places without a sufficient number of highly skilled embryologists or large well-endowed laboratories, thus reducing overall costs.

Lastly, this Example validates the potential of an automated embryology lab-on-a-chip device for the denudation of oocytes.

### Supplementary Notes

### Morphokinetics

Time-lapse data for full pre-implantation embryo development from fertilized oocytes denuded by manual pipetting (control), 80 MHz, or 200 MHz SAW. Exact post-insemination timing into 2nd polar body extrusion (tPB2), pronuclei appearance and fading (tPNa and tPNf), cleavage in to 2-cell to 8-cell (t2 to t8), morula compaction (tM), early blastulation (tSB), full blastocyst development (tB) and hatching (tHB) were recorded and compared.

### Calculation of Body Force on the Liquid

According to the theory of acoustic streaming a sound wave damps upon entering a liquid through relaxational dissipation.⁹³ Consequently, a volumetric force, as a function of frequency-dependent absorption coefficients, acts on the body of the liquid in the direction of wave propagation. For example, a plane wave propagating in the x-direction with an amplitude of ^{A} and damping length of lₐ can be described as

$$U = {Ae}^{\frac{x}{{l}_{a}}} cos ( ω t - kx )$$

the force is then given by

$${F}_{x} = {ρ}_{0} {A}^{2} {e}^{\frac{2 x}{{l}_{a}}} / {l}_{a}$$

in which amplitude can be calculated from power and surface area from

$$P = \frac{1}{2} c {ρ}_{0} {A}^{2} n \cdot s .$$

By substitution, the force in the x-direction simplifies to

$${F}_{x} = \frac{2 P}{{cSl}_{a} cos ( θ )} {e}^{\frac{2 x}{{l}_{a}}}$$

which reveals a linear relationship between force and acoustic power.⁵⁰ Due to the linear relationship of electric power and acoustic power, with Stokes equations governing the fluid flow, a linear relationship between the fluid flow and applied power is expected. Assuming the primary mechanism of denudation is from the shear of the drag forces, such linearity is observed between the denudation efficiency as a function of applied power. (FIG. 6A)

### SAW Denudation Module Fabrication

### SAW Chip Fabrication

Applicant chose 128° Y-cut lithium Niobate (K2=5.3%) with a reduced pyroelectric coefficient as substrate material because of its higher electromechanical coefficient. Applicant used a lift-off process for transferring patterns on top of the substrate. A thin film of Ti/Au (200 nm/10 nm) was deposited using the E-beam evaporation process on a substrate patterned with a negative tone photoresist (nLoF-2020). After lift-off cleaning, the substrate was diced and glued to the printed circuit board (PCB) using a very thin epoxy layer. After securing the SAW substrate on the PCB, electrical connections were made using a wire bonding technique. In order to prevent unwanted wire detachments during the denudation procedure and minimize spurious electrical fields, Applicant covered the wire bonds with potting epoxy. The potting epoxy was intentionally applied very close to the end of its working time window (at high viscosity) to minimize its spread on the substrate. The SAW chip fabrication steps are illustrated in FIG. 10B.

### Micro-Well Fabrication Steps

The micro-milling technique is used for the fabrication of the layers necessary for microwell production. Due to the wide angle of the microwell attached to the substrate, it is not possible to remove the PDMS layer from the substrate. As such, the microwell master is fabricated in two pieces that could be assembled like Lego pieces as illustrated in FIG. 10A. After assembly, the PDMS is poured on the master substrate until it is slightly lower than the microwell height such that the microwell top surface remains exposed to the air. This technique allows for the removal of the microwell piece without puncturing the PDMS layer and allows the pieces to be reusable. It is worth mentioning that theoretically, the height of the fringe that defines the bottom geometry of the microwell should be the same height as the sections covering the IDTs to assure complete sealing. However, even when the height of the fringe is slightly smaller than the gap covering the IDT sections, preventing it from touching the substrate, the resulting structure automatically prevents the medium leakage by creating an expansion zone that acts like microfluidic stop valves.⁹⁴ This works to our advantage, making the microfabrication process less sensitive to slight height variations. However, applying higher actuation power >30 dBm can overcome the Laplace pressure and draw the liquid over the IDTs, leading to actuation attenuation.⁹⁵

### Electrical Characterization of SAW

Applicant chose an ultrasound resonant design for x and y directions to also benefit from the Q factor to increase the efficiency (scale factor) of the device. In addition, Applicant also added the thin-film metallic short reflector gratings in the x-direction to confine the main ultrasound beam to the acoustic cavity and reduce leakage losses. This easy to fabricate method introduces small acoustic impedances at the metal deposited locations and if their number reaches the critical reflecting element number (NR), they can reflect most of the incident waves back to the acoustic cavity. As such >300 reflecting elements calculated based on the Sitting method⁹⁶ were added to reflect most of the incident wave back. Applicant measured the resonant frequency of the devices by measuring the SAW scattering parameters using a Keysight E5061B analyzer. The S21 parameter, comparing the transmission from input to output for each direction, is shown in FIG. 11B. Resonant frequency shift measured for x directions due to temperature variation either due to fluctuation induced by the attached heater or due to the thermal effects induced by the SAW itself is shown in FIG. 11A. The corresponding resonant frequencies, wavelengths, and damping lengths are listed in Table 1.

### Electrical Effects

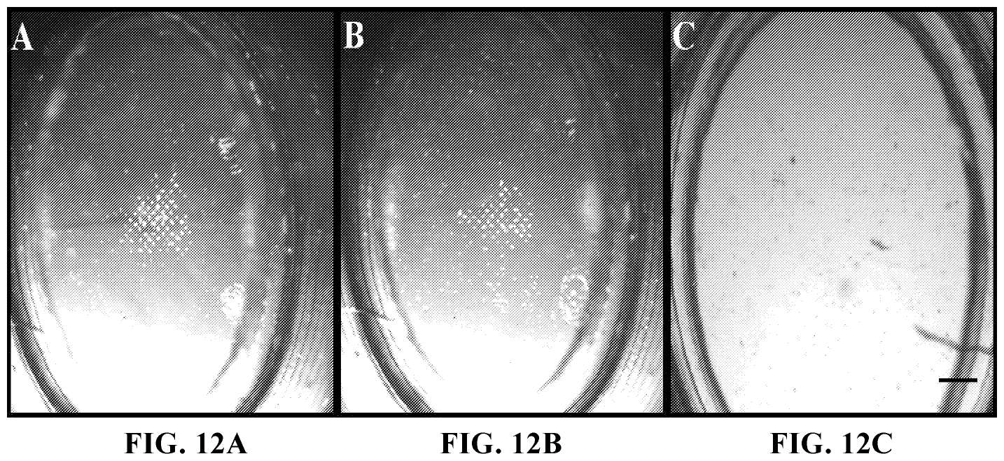

FIG. 12A-12C show various electrical effects.

### Lithium Niobate Birefringence and Image Quality

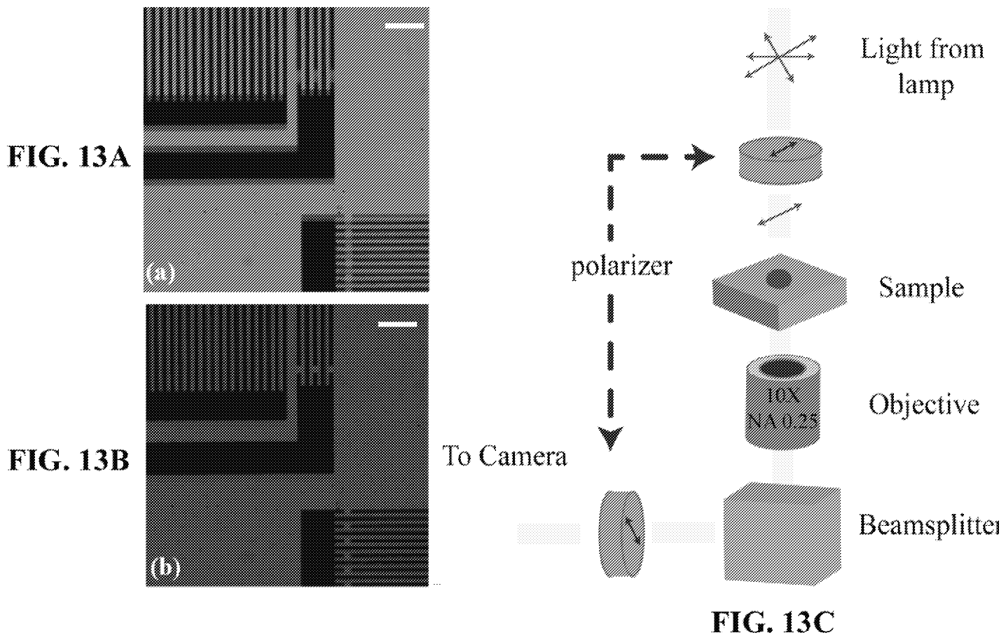

The lithium niobate used as the piezo substrate of the acoustic denudation module is a negative uniaxial crystal (double refracting crystal). The axial birefringence in lithium niobate crystals induces splitting in transmission optical images in an inverted microscope setup, resulting in the creation of twin images.^{97,91} Using a polarizer to eliminate one of the twin images results in light scarceness, decreasing the light intensity by half and lowering the image quality as can be seen in FIG. 13A-13C. Surprisingly, although the amount of decreased light can be compensated for by increasing the amount of the incoming light, the image quality remains lower compared to the glass substrate, eventually compromising the image processing steps. As such, the same denudation mechanism can be applied using other actuation mechanisms. For example, using BAW mode ultrasonics with piezoelectric ceramics or electromagnetic actuation of a transparent membrane with embedded micromagnets, which eliminates the need for special substrates resulting in even lower fabrication costs.

### Movies

Incorporated herein by reference are Movies S1-S8 of Mokhtare et al., Lab Chip, 2022, 777-792. Movie captions are as follows:

Movie S1. Flow patterns in “dry” state devices with circular and elliptical microwells. Fluidic patterns revealed by mixing a high density fluidic (OptoPrep media) with deionized water. The scale bar is 800 microns. Foldings are clearly seen upon shifting the resonant frequency from x to y-direction.

Movie S2. CW vs SFM mode signal actuation of a “dry” state device with an elliptical base microwell. In CW mode, 15-micron particles assemble in specific patterns while applying an SFM mode signal to achieve complete mixing inside the microwell without creating any specific patterns.

Movie S3. 100-microns particles' trajectories in a “dry” state device actuated with (Movie S3A) a CW signal in the x-direction. (Movie S3B) an SFM signal. 100-microns particles' trajectories in a “wet” state device actuated with (Movie S3A) a CW signal in the x-direction. (Movie S3B) cell trajectories in a “wet” state device derived with an SFM signal.

Movie S4. Representative demonstration of in vitro testing protocol. Movie S4A-D shows 10 seconds of the operation from loading to complete denudation consecutively.

Movie S5. Representative demonstration of in vivo testing protocol for both “dry” and “wet” state devices showing complete denudation of multiple oocytes.

Movie S6. Representative demonstration of a big cluster of fresh COC denudation on a “wet” state device and gradual detachment of single COCs from the cluster.

Movie S7. Representative time-lapse imaging videos of oocytes denuded with both “dry” and “wet” state devices for morphokinetics embryo development evaluation.

Movie S8. Representative Piezo-ICSI performance on denuded oocytes.

### REFERENCES FOR EXAMPLE 1

1. L. F. Rienzi, R. Maggiulli and F. M. Ubaldi, in In VitroFertilization: A Textbook of Current and Emerging Methods and Devices, ed. Z. P. Nagy, A. C. Varghese and A. Agarwal, Springer International Publishing, Cham, 2019, pp. 133-145.
2. T. Ebner, in In Vitro Fertilization: A Textbook of Current and Emerging Methods and Devices, ed. Z. P. Nagy, A. C. Varghese and A. Agarwal, Springer International Publishing, Cham, 2019, pp. 471-479.
3. ESHRE Capri Workshop Group, Hum. Reprod. Update, 2007, 13, 515-526.
4. A. R. Thornhill, C. E. de Die-Smulders, J. P. Geraedts, J. C. Harper, G. L. Harton, S. A. Lavery, C. Moutou, M. D. Robinson, A. G. Schmutzler, P. N. Scriven, K. D. Sermon, L. Wilton and ESHRE PGD Consortium, Hum. Reprod., 2005, 20, 35-48.
5. Germ Cell Protocols: Volume 2: Molecular Embryo Analysis, Live Imaging, Transgenesis, and Cloning, ed. H. Schatten, Humana Press, 2004.
6. K. Meyer, in The Enzymes, ed. P. D. Boyer, Academic Press, 1971, vol. 5, pp. 307-320.
7. Y. Ishizuka, T. Takeo, S. Nakao, H. Yoshimoto, Y. Hirose, Y. Sakai, Y. Horikoshi, S. Takeuji, S. Tsuchiyama and N. Nakagata, J. Reprod. Dev., 2014, 60, 454-459.
8. B. R. L. de Moura, M. C. A. Gurgel, S. P. Machado, P. A. Marques, J. R. Rolim, M. C. de Lima and L. L. Salgueiro, JBRA Assist. Reprod., 2017, 21, 27-30.
9. L. Rienzi, F. Ubaldi, F. Martinez, M. Iacobelli, M. G. Minasi, S. Ferrero, J. Tesarik and E. Greco, Hum. Reprod., 2003, 18, 1289-1293.
10. 10 S. Cooke, J. P. P. Tyler and G. L. Driscoll, Hum. Reprod., 2003, 18, 2397-2405.
11. C. Patrat, A. Kaffel, L. Delaroche, J. Guibert, P. Jouannet, S. Epelboin, D. De Ziegler, J.-P. Wolf and P. Fauque, Obstet. Gynecol. Int., 2012, 2012, 403531.
12. D. Beebe, M. Wheeler, H. Zeringue, E. Walters and S. Raty, Theriogenology, 2002, 57, 125-135.
13. Applications of Microfluidic Systems in Biology and Medicine, ed. M. Tokeshi, Springer, Singapore, 2019.
14. M. Yaghoobi, M. Azizi, A. Mokhtare and A. Abbaspourrad, Lab Chip, 2021, 21, 2791-2804.
15. H. C. Zeringue and D. J. Beebe, in Germ Cell Protocols, Springer, 2004, pp. 365-373.
16. H. C. Zeringue, J. J. Rutledge and D. J. Beebe, Lab Chip, 2005, 5, 86-90.
17.q L. Weng, G. Y. Lee, J. Liu, R. Kapur, T. L. Toth and M. Toner, Lab Chip, 2018, 18, 3892-3902.
18. A. Mokhtare, P. Xie, A. Abbaspourrad, Z. Rosenwaks and G. GREAT CLARENDON ST, OXFORD OX2 6DP, ENGLAND, 2020, vol. 35, pp. 71-72.
19. A. Mokhtare, P. Xie, A. Abbaspourrad, Z. Rosenwaks and G. D. Palermo, Fertil. Steril., 2020, 114, e76.
20. J. E. Swain, D. Lai, S. Takayama and G. D. Smith, Lab Chip, 2013, 13, 1213-1224.
21. A. Ozcelik, J. Rufo, F. Guo, Y. Gu, P. Li, J. Lata and T. J. Huang, Nat. Methods, 2018, 15, 1021-1028.
22. Z. Chen, X. Liu, M. Kojima, Q. Huang and T. Arai, NATO Adv. Study Inst. Ser., Ser. E, 2020, 10, 1260.
23. W. Connacher, N. Zhang, A. Huang, J. Mei, S. Zhang, T. Gopesh and J. Friend, Lab Chip, 2018, 18, 1952-1996.
24. J. P. Shelby and D. T. Chiu, Lab Chip, 2004, 4, 168-170.
25. A. Fakhfouri, C. Devendran, T. Albrecht, D. J. Collins, A. Winkler, H. Schmidt and A. Neild, Lab Chip, 2018, 18, 2214-2224.
26. J. Friend and L. Y. Yeo, Rev. Mod. Phys., 2011, 83, 647-704.
27. G. Destgeer and H. J. Sung, Lab Chip, 2015, 15, 2722-2738.
28. J. Hultström, O. Manneberg, K. Dopf, H. M. Hertz, H. Brismar and M. Wiklund, Ultrasound Med. Biol., 2007, 33, 145-151.
29. F. S. Foster, C. J. Pavlin, K. A. Harasiewicz, D. A. Christopher and D. H. Turnbull, Ultrasound Med. Biol., 2000, 26, 1-27.
30. M. A. Burguillos, C. Magnusson, M. Nordin, A. Lenshof, P. Augustsson, M. J. Hansson, E. Elmér, H. Lilja, P. Brundin, T. Laurell and T. Deierborg, PLoS One, 2013, 8, e64233.
31. V. Marx, Nat. Methods, 2015, 12, 41-44.
32. M. Wiklund, Lab Chip, 2012, 12, 2018-2028.
33. M. Mahadevan, K. Chalder, D. Wiseman, A. Leader and P. J. Taylor, J. In Vitro Fert. Embryo Transf., 1987, 4, 277-280.
34. S. Lenz, J. G. Lauritsen and M. Kjellow, Lancet, 1981, 317, 1163-1164.
35. A. Cardinale, R. Lagalla, V. Giambanco and F. Aragona,

### Ultrasonics, 1991, 29, 261-263.

36. U. S. Food, D. Administration and Others, Rockville, MD: FDA. 37 D. L. Miller, N. B. Smith, M. R. Bailey, G. J. Czarnota, K. Hynynen, I. R. S. Makin and Bioeffects Committee of the American Institute of Ultrasound in Medicine, J. Ultrasound Med., 2012, 31, 623-634.
38. Z. Xin, G. Lin, H. Lei, T. F. Lue and Y. Guo, Transl. Androl.

### Urol., 2016, 5, 255-266.

39. J. S. Abramowicz, in Ultrasound Imaging in Reproductive Medicine: Advances in Infertility Work-up, Treatment and ART, ed. L. A. Stadtmauer and I. Tur-Kaspa, Springer International Publishing, Cham, 2019, pp. 3-17.
40. M. A. Ghanem, A. D. Maxwell, Y.-N. Wang, B. W. Cunitz, V. A. Khokhlova, O. A. Sapozhnikov and M. R. Bailey, Proc. Natl. Acad. Sci. U.S.A, 2020, 117, 16848-16855.
41. USPTO, US Patent, 20200095529:A1, 2020.
42. J. Park, B. H. Ha, G. Destgeer, J. H. Jung and H. J. Sung, RSC Adv., 2016, 6, 33937-33944.
43. M. Wiklund, R. Green and M. Ohlin, Lab Chip, 2012, 12, 2438-2451.
44. M. B. Dentry, L. Y. Yeo and J. R. Friend, Phys. Rev. E: Stat., Nonlinear, Soft Matter Phys., 2014, 89, 013203.
45. P. Zhang, H. Bachman, A. Ozcelik and T. J. Huang, Annu. Rev. Anal. Chem., 2020, 13, 17-43.
46. M. Gedge and M. Hill, Lab Chip, 2012, 12, 2998-3007.
47 A. R. Rezk, L. Y. Yeo and J. R. Friend, Langmuir, 2014, 30, 11243-11247.
48. G. Destgeer, J. H. Jung, J. Park, H. Ahmed and H. J. Sung, Anal. Chem., 2017, 89, 736-744.
49. P. Zhang, C. Chen, X. Su, J. Mai, Y. Gu, Z. Tian, H. Zhu, Z. Zhong, H. Fu, S. Yang, K. Chakrabarty and T. J. Huang, Sci. Adv., 2020, 6, eaba0606.
50. L. Schmid, A. Wixforth, D. A. Weitz and T. Franke, Microfluid. Nanofluid., 2012, 12, 229-235.
51. G. T. Silva, J. H. Lopes, J. P. Leão-Neto, M. K. Nichols and B. W. Drinkwater, Phys. Rev. Appl., 2019, 11, 054044.
52. B. W. Drinkwater, Appl. Phys. Lett., 2020, 117, 180501.
53. J. Bennes, S. Ballandras and F. Chérioux, Appl. Surf. Sci., 2008, 255, 1796-1800.
54. A. Winkler, R. Brünig, C. Faust, R. Weser and H. Schmidt, Sens. Actuators, A, 2016, 247, 259-268.
55. J. E. Michaels, S. J. Lee, A. J. Croxford and P. D. Wilcox, Ultrasonics, 2013, 53, 265-270.
56. Y. J. Montagut, J. V. Garcia, Y. Jiménez, C. March, A. Montoya and A. Arnau, Rev. Sci. Instrum., 2011, 82, 064702.
57. S. Assous, J. Rees, M. Lovell, L. Linnett and D. Gunn, Advances in Piezoelectric Transducers, 2011, p. 75.
58. R. T. Smith and F. S. Welsh, J. Appl. Phys., 1971, 42, 2219-2230.
59. R. J. Shilton, M. Travagliati, F. Beltram and M. Cecchini, Adv. Mater., 2014, 26, 4941-4946.
60. G. Destgeer, B. Ha, J. Park and H. J. Sung, Anal. Chem., 2016, 88, 3976-3981.
61. G. Destgeer, H. Cho, B. H. Ha, J. H. Jung, J. Park and H. J. Sung, Lab Chip, 2016, 16, 660-667.
62 T. Hasegawa and K. Yosioka, J. Acoust. Soc. Am., 1969, 46, 1139-1143.
63. T. Hasegawa and K. Yosioka, J. Acoust. Soc. Am., 1975, 58, 581-585.
64. A. A. Doinikov, Recent research developments in acoustics, 2003, vol. 1, pp. 39-67.
65. K. S. Wong, L. Lee, Y. M. Hung, L. Y. Yeo and M. K. Tan, Anal. Chem., 2019, 91, 12358-12368.
66. T. R. Nelson, J. B. Fowlkes, J. S. Abramowicz and C. C. Church, J. Ultrasound Med., 2009, 28, 139-150.
67. C. C. Church, C. Labuda and K. Nightingale, Ultrasound Med. Biol., 2015, 41, 472-485.
68. H. Ando, L. B. Feril Jr, T. Kondo, Y. Tabuchi, R. Ogawa, Q.-L. Zhao, Z.-G. Cui, S.-I. Umemura, H. Yoshikawa and T. Misaki, Cancer Lett., 2006, 242, 37-45.
69. Y. Feng, Z. Tian and M. Wan, J. Ultrasound Med., 2010, 29, 963-974.
70. K. H. Ng, J. Med. Ultrasound, 2002, 10, 5-9.
71. E. Lucena and H. Moreno-Ortiz, in Development of In Vitro Maturation for Human Oocytes: Natural and Mild Approaches to Clinical Infertility Treatment, ed. R.-C. Chian, G. Nargund and J. Y. J. Huang, Springer International Publishing, Cham, 2017, pp. 267-279.
72. P. S. Balasubramanian, A. Singh, C. Xu and A. Lal, Sci. Rep., 2020, 10, 3075.
73. L. E. Houston, A. O. Odibo and G. A. Macones, Prenatal Diagn., 2009, 29, 1204-1212.
74. M. W. Miller, W. L. Nyborg, W. C. Dewey, M. J. Edwards, J. S. Abramowicz and A. A. Brayman, Int. J. Hyperthermia, 2002, 18, 361-384.
75. Z. Izadifar, P. Babyn and D. Chapman, Ultrasound Med. Biol., 2017,43, 1085-1104.
76. R. J. Shilton, V. Mattoli, M. Travagliati, M. Agostini, A. Desii, F. Beltram and M. Cecchini, Adv. Funct. Mater., 2015, 25, 5895-5901.
77. T. Zheng, C. Wang, Q. Hu and S. Wei, Appl. Phys. Lett., 2018, 112, 233702.
78. M. Baudoin, J.-L. Thomas, R. A. Sahely, J.-C. Gerbedoen, Z. Gong, A. Sivery, O. B. Matar, N. Smagin, P. Favreau and A. Vlandas, Nat. Commun., 2020, 11, 4244.
79. S. Collignon, O. Manor and J. Friend, Adv. Funct. Mater., 2018, 28, 1704359.
80. J. Kubanek, J. Shi, J. Marsh, D. Chen, C. Deng and J. Cui, Sci. Rep., 2016, 6, 24170.
81. A. Lervik, S. Kjelstrup and H. Qian, Phys. Chem. Chem. Phys., 2015, 17, 1317-1324.
82. K. Lange, B. E. Rapp and M. Rapp, Anal. Bioanal. Chem., 2008, 391, 1509-1519.
83. X. H. Kong, C. Deneke, H. Schmidt, D. J. Thurmer, H. X. Ji, M. Bauer and O. G. Schmidt, Appl. Phys. Lett., 2010, 96, 134105.
84. S. H. Weinberg, PLoS One, 2013, 8, e81402.
85. A. R. Rezk, H. Ahmed, T. L. Brain, J. O. Castro, M. K. Tan, J. Langley, N. Cox, J. Mondal, W. Li, M. Ashokkumar and L. Y. Yeo, J. Phys. Chem. Lett., 2020, 11, 4655-4661.
86. G. Peyre, Comput. Sci. Eng., 2011, 13, 94-97.
87. R. S. Sengar, A. K. Upadhyay, M. Singh and V. M. Gadre, Biomed. Signal Process. Control, 2016, 25, 62-75.
88. R. S. Sengar, A. K. Upadhyay, M. Singh and V. M. Gadre, in 2012 National Conference on Communications (NCC), 2012, pp. 1-5, ieeexplore.ieee.org.
89. A. K. Upadhyay and A. K. Upadhyay, Wavelet Based Image Segmentation, https://www.mathworks.com/matlabcentral/fileexchange/48610-wavelet-based-image-segmentation, (accessed Mar. 8, 2021).
90. J. Costa, S. McHugh, N. Rice, P. J. Turner, B. A. Willemsen, N. O.

Fenzi, R. B. Hammond, J. D. Ha, C. H. Lee and T. Sato, in 2017 IEEE International Ultrasonics Symposium (IUS), 2017, pp. 1-4.
91. Home—Embryotech Laboratories Inc, https://embryotech.com/, (accessed Mar. 4, 2021).
92. N. Yoshida and A. C. F. Perry, Nat. Protoc., 2007, 2, 296-304. 93 M. Green, S. Bass and B. Spear, BioTechniques, 2009, 47, 919-924.
93. S. J. Lighthill, J. Sound Vib., 1978, 61, 391-418.
94. A. Olanrewaju, M. Beaugrand, M. Yafia and D. % J L. on a. C. Juncker, 2018, 18, 2323-2347.
95. A. R. Rezk, O. Manor, J. R. Friend and L. Y. Yeo, Nat. Commun., 2012, 3, 1167.
96. E. K. Sittig and G. A. Coquin, IEEE Transactions on Sonics and Ultrasonics, 1968, 15, 111-118.
97. L. Arizmendi, Physica Status Solidi A Appl. Res., 2004, 201, 253-283.
98. A. A. Nawaz, M. Urbanska, M. Herbig, M. Nötzel, M. Kräter, P. Rosendahl, C. Herold, N. Toepfner, M. Kubánková, R. Goswami, S. Abuhattum, F. Reichel, P. Müller, A. Taubenberger, S. Girardo, A. Jacobi and J. Guck, Nat. Methods, 2020, 17, 595-599.

### Example 2

Embodiments of the device described herein can provide a chip scale acoustic module consisting of a Lithium niobate (LiNbO₃) piezoelectric substrate with patterned interdigitated transducers that can deliver nontoxic Megahertz ultrasonic wave to a sessile droplet including the COCs. The delivered acoustic energy results in acoustic actuation of the droplet contents and induces cumulus denudation in a noncontact method preventing any unwanted mechanical stress from pipetting.

Embodiments of the devices, systems, and/or methods described herein can achieve COC denudation solely by fluid induced shear stresses where its magnitude can be controlled by acoustic energy. Embodiments allow for manual and/or automatic loading that can be optionally controlled with a microcontroller and computer. Embodiments of the devices, systems, and/or methods described herein can be configured to maintain a constant fluid environment and reduce manual handling steps. Embodiments of the devices, systems, and/or methods described herein can significantly reduce the time, labor while granting quality control and minimizing inter- and intra-operator and other variabilities. Embodiments of the devices, systems, and/or methods described herein can be disposable. Embodiments of the devices, systems, components thereof, and/or methods described herein can be configured for multiple usage, which can significantly reduce input costs and waste.

Some features, advantages, and/or benefits of embodiments of the device, systems, and methods herein, can include, without limitation, denudation of oocyte without any physical contact with any obstacles, reduced shear and mechanical stress in comparison to existing platforms and manual pipetting, easy loading and retrieval from the droplets, greater flexibility levels similar to manual pipetting, the procedure can be finely tuned to achieve denudation based on desired parameters, the device and/or system can be easily controlled through a programmable microcontroller and graphical user interface, the device and/or system can allow for continuous and/or visual tracking of individual COCs as they are processing, a reduced probability of loss less during washing and transferring steps, improved reproducibility of denudation over existing methods and/or devices.

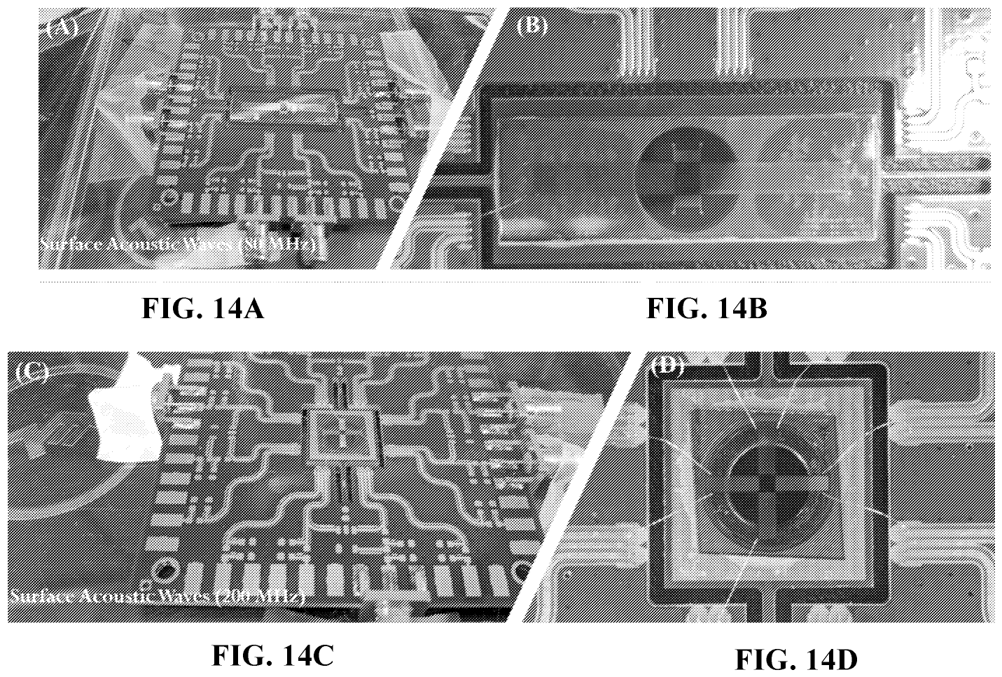

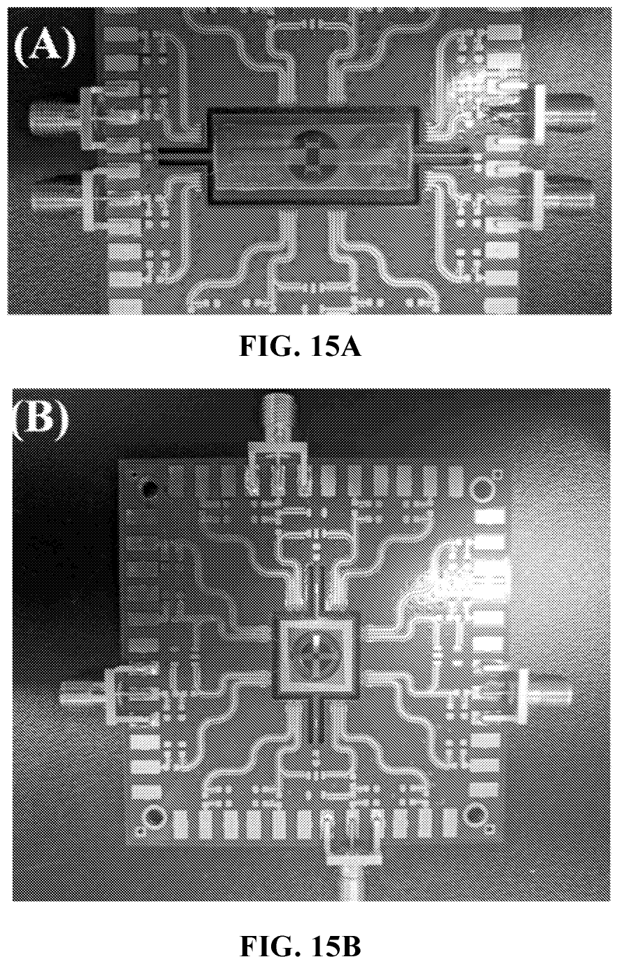

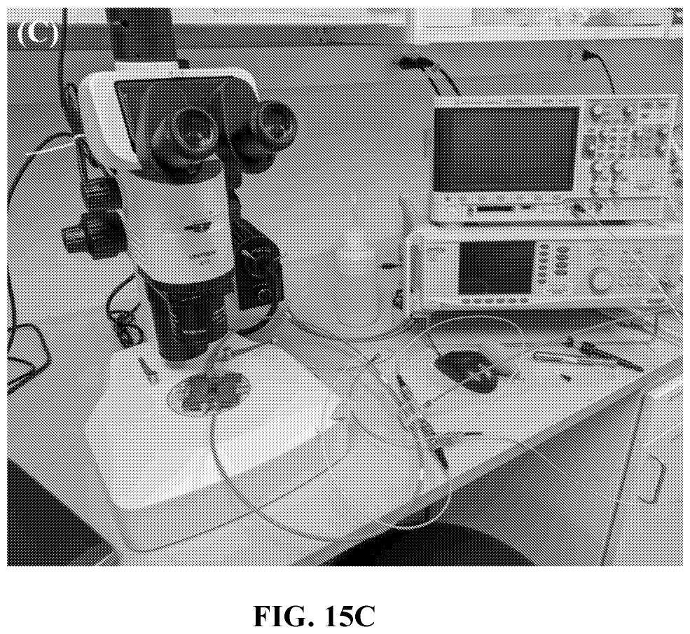

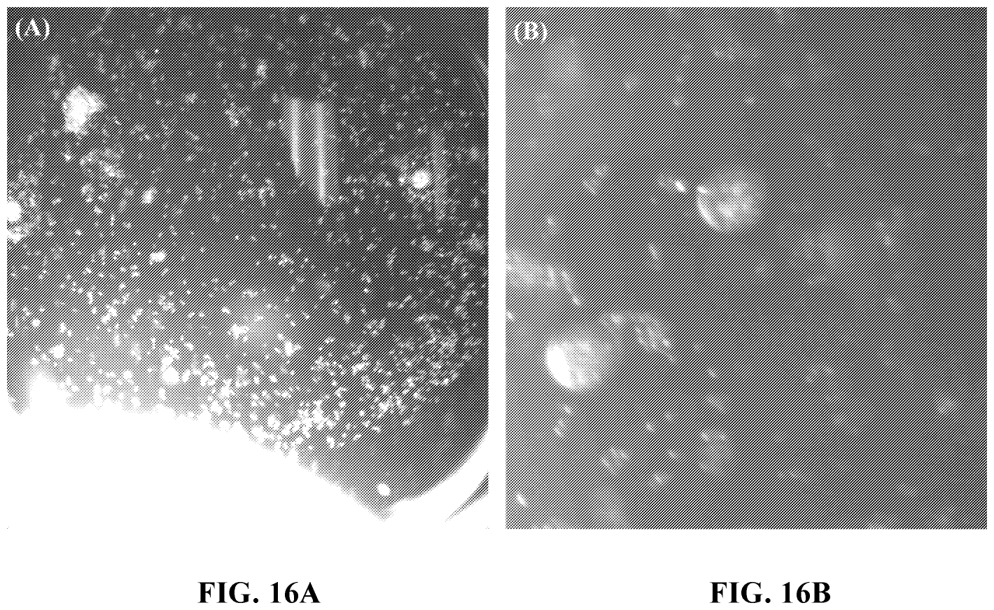

Exemplary devices and use are shown in FIGS. 14A-14D, FIGS. 15A-15C, and FIGS. 16A-16B.

### Example 3

Currently, there are two major methods for cumulus removal. One method involves the use of properly sized glass pipettes. The technician flushes the COCs into and out of the pipette tip to mechanically remove the cumulus. In the second method, the cumulus and corona cells are removed by exposure of the COCs to a combined enzymatic and mechanical treatment that causes the breakdown of the matrix surrounding COCs and disperses the cumulus cells from oocytes. Hyaluronidase alone does not remove the cumulus and is typically used to aid the mechanical pipetting. During the entire procedure, efforts should be made to minimize risks such as oocyte parthenogenetic activation, PB1 and Miotic spindle dislocation, zona pellucida fracture and oocyte degeneration as a result of mechanical stress.

As an alternative to conventional methods, acoustofluidic methods can be employed for oocyte preparation. Acoustofluidics is a growing field where the science of micro-manipulation of fluids and microparticles by sound waves is used to study biological systems at the micro- and nanoscales. In ART, a single assisted reproduction therapy cycle ultrasound is currently used for retrieval of oocytes from the ovaries and in the last step of transferring a single or multiple embryos back to the uterus. The fluid flow induce by sound waves also known as acoustic streaming and acoustic radiation force on the cells can be used and controlled for oocyte denudation.

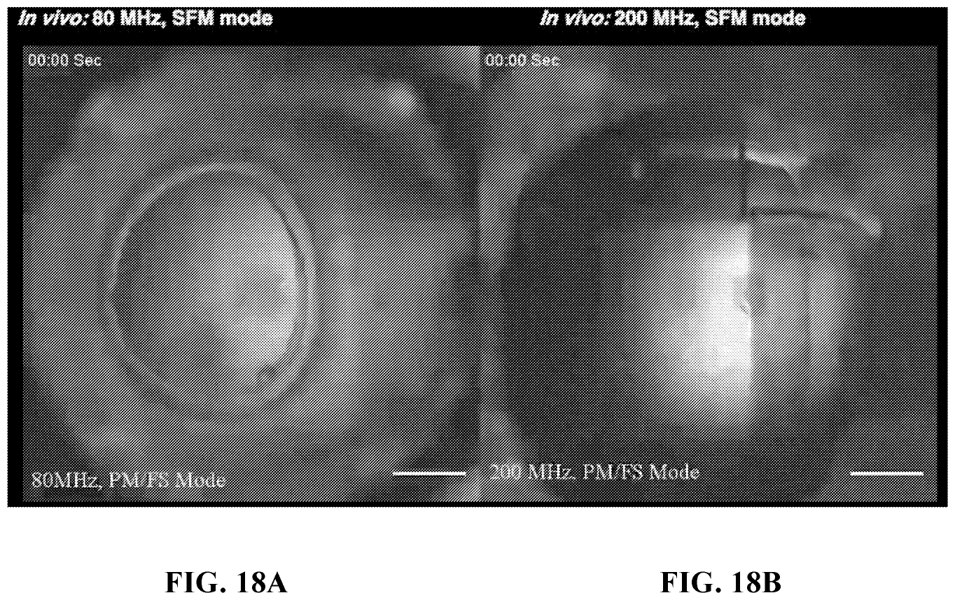

This Example describes a device that is composed of two parts: (1) A piezo electric substrate mounted on a PBC that converts the electrical signals to mechanical waves; and (2) disposable microwells that are placed on the substrate for housing the intact cumulus oocyte complexes. See e.g., FIG. 18.

The Interdigitated transducers (or IDTs in short) are either placed outside or inside the microwell in two configurations. When the IDTs are outside the microwell, Applicant refers to it as a “dry” state device since they are not in contact with the medium and it operates at 80 MHz frequency. While, in the second configuration the IDTs are embedded at the bottom of the microwell and it operates at 200 MHz. A simulation from Monash University clearly shows how surface acoustic waves are generated on the substrate at the bottom of the microwells.

Upon entering the microwell, these wave induce fluid flow and impose forces in the cell and eventually disperse the cumulus cells.

In order the investigate the safety and efficiently of our method, Applicant developed two protocols for testing the device. See FIG. 8A-8B. In the in vitro protocol, we tested and optimized our device parameters using commercially available cryopreserved COC.

Then, using the optimized conditions Applicant carried the denudation procedure on COCs that were surgically removed from mouse ovaries, followed by investigation of the development potential of the resulting oocytes.

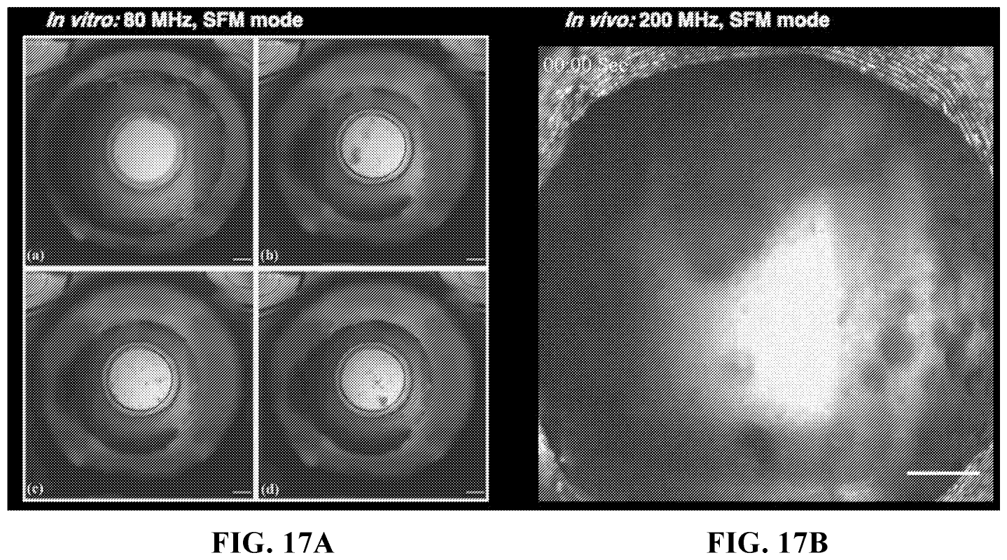

The movie captures shown in FIG. 17A-17B show the working mechanism of the “dry state 80 MHz device. Top left shows the first 10 seconds of the working device, the top right shows the second 10 seconds or from second 10 to 20 and so on. During the 40 seconds the device is able to reach to almost complete denudation of the frozen cell. The movie capture of FIG. 17A-17B show to the first 7 seconds of the “dry state” device tested with the fresh cells that was surgically removed from the mouse ovary. As you can see, during the first few seconds the COCs are detached from the initial cluster, and followed by the cumulus deprivation similar the in vitro protocol.

The movie captures if FIG. 18A-18B show both devices tested using fresh COCs by an experienced embryologist that had no prior experience working with the devices. By pausing and visual assessment of the cells during the procedure they either decided to continue or stop the denudation procedure and retrieve the oocytes from the microwell to a washing droplet on a petri-dish for injection.

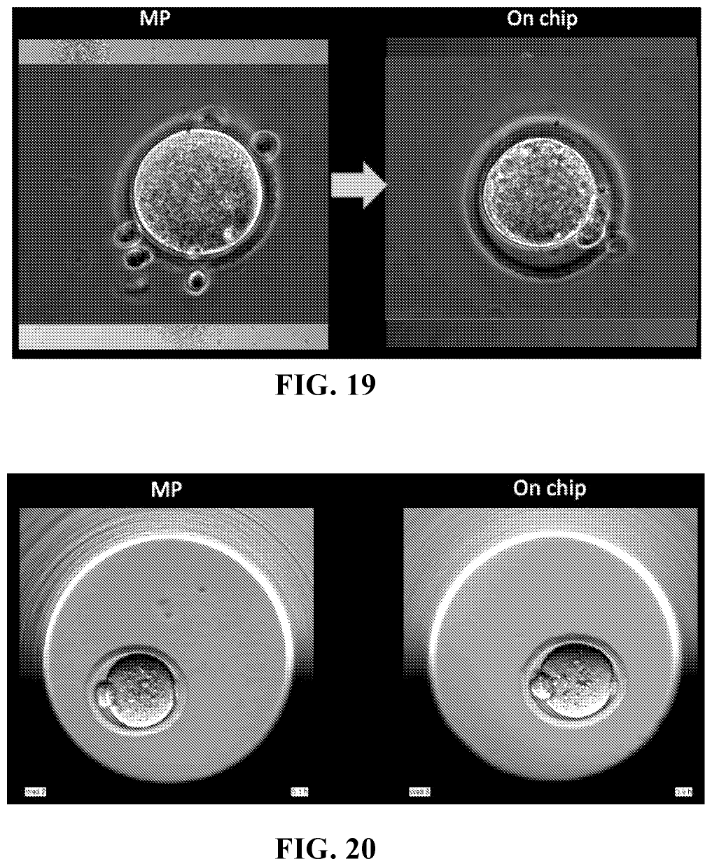

The movie captures of FIG. 19 show some details on how we obtain cumulus oocyte complexes. A cohort of oocytes was retrieved from superovulated B6D2F1 adult mice. The oocytes were exposed to 0.5 mg/ml HA and M2 medium inside the microwell and immediately exposed to acoustofluidic field. The performance of the microfluidic chip and conventional method are very similar.

FIG. 20 shows is the entire development of both control and ultrasound exposed zygotes side by side Starting from hour 5 after manipulation, syngamy occurred at around hour 14, first cleavage occurred right after, further timing of cleavage into 4-cell and 8-cell are comparable. The development into morula and blastocysts are also similar. The ultrasound exposed zygotes resulted a 71.2.% blastulation rate, slightly higher than the control but not statistically significant.

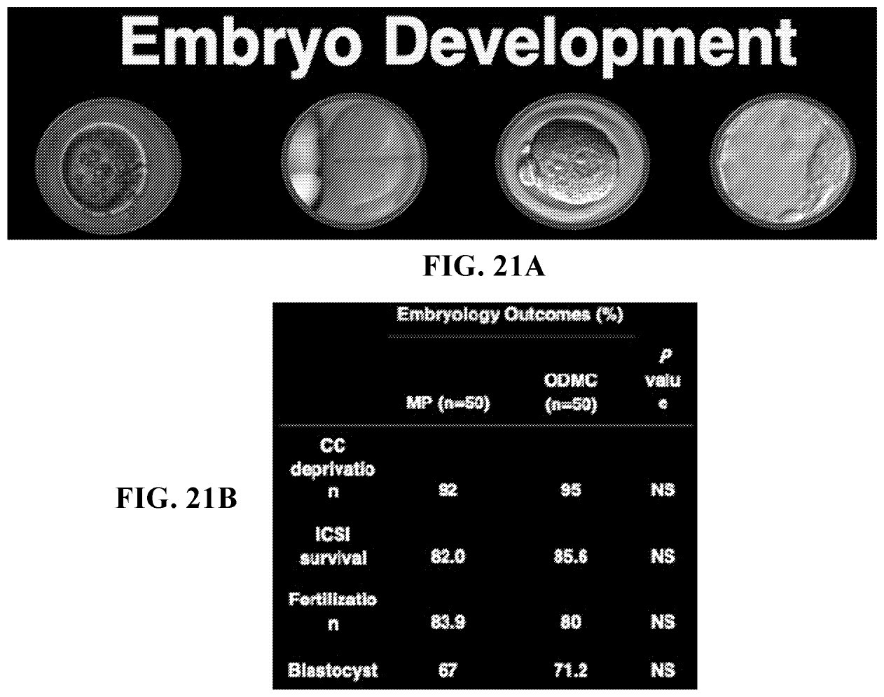

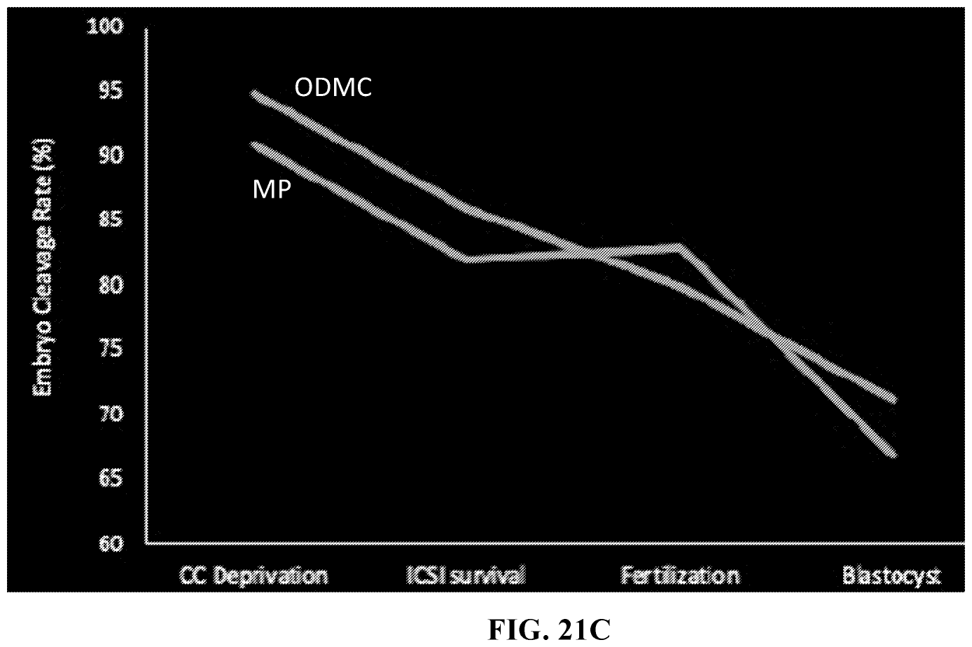

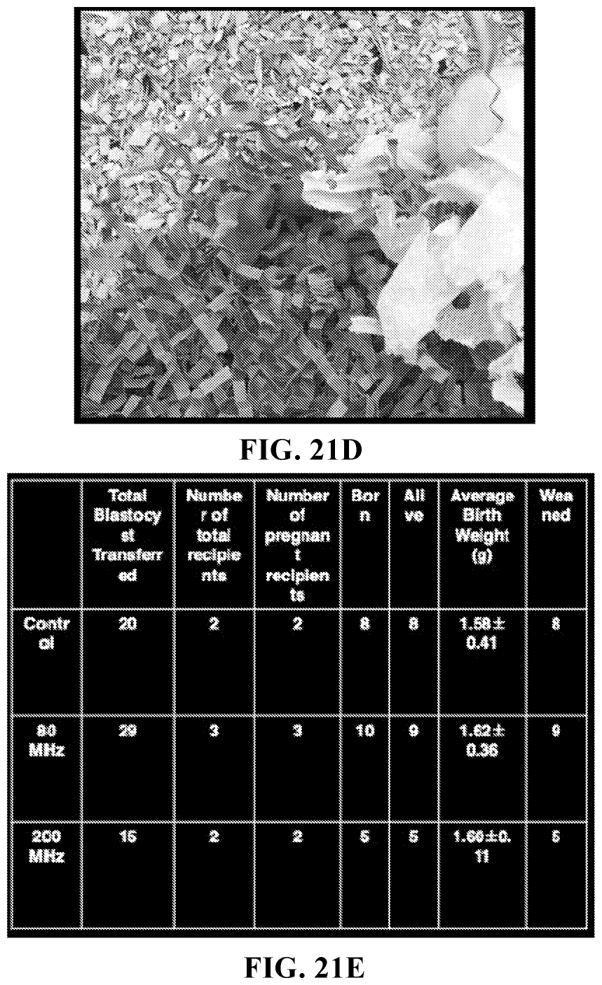

FIG. 21A-21E demonstrate the results of overall embryo development. In this experiment, the developments of ultrasound exposed zygotes into 2-cell, 4-cell, morula and blastocyst are comparable to control zygotes. FIG. 21A-21C show a visual presentation comparing cleavage between control and ultrasound exposed zygotes. FIG. 21D-21E show results of overall development. After transferring the resulting blastocysts into recipient mice, 9 live births were achieved from the 80-MHz group, while 5 were achieved from the 200-MHz group. All pups were weaned to date without compromised development and are fertile.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. An acoustofluidic contactless sample agitator comprising:
   a piezoelectric substrate capable of propagating a surface acoustic wave;
   a chamber configured to contain a volume of a liquid, the chamber comprising one or more walls forming the sides of the chamber and a bottom comprising an opening;
   at least two pairs of orthogonal interdigitated transducers (IDTs) deposited on a surface of the piezoelectric substrate; and
   wherein the piezoelectric substrate is coupled to the chamber such that the two pairs of orthogonal IDTs are arranged about the periphery of the opening on the bottom of the chamber such that each IDT in each IDT pair is opposite each other on different sides of the opening, that the two IDT pairs are substantially perpendicular to each other about the periphery of the opening, and that the opening is substantially centered between the two IDT pairs, and
   wherein, together, the two pairs of IDTs are configured to produce tunable orthogonal surface acoustic waves in the piezoelectric substrate effective to produce acoustic streaming and/or acoustic radiation force within a liquid present in the chamber sufficient to mix the liquid present inside the chamber and/or agitate one or more particles, cells, and/or complexes thereof present in the liquid without trapping the one or more particles, cells, and/or complexes thereof within the liquid.
2. The acoustofluidic contactless sample agitator of aspect 1, wherein chamber is detachably coupled to the piezoelectric substrate.
3. The acoustofluidic contactless sample agitator of any one of aspects 1-2, wherein the two pairs of orthogonal IDTs are arranged about the periphery of the opening such that they do not extend into the opening and do not contact a liquid present in the chamber.
4. The acoustofluidic contactless sample agitator of aspect 1, wherein the two pairs of orthogonal IDTs are arranged about the periphery of the opening such that a portion of each IDT extends into the opening such that the portion contacts a liquid present in the chamber.
5. The acoustofluidic contactless sample agitator of aspect 4, wherein none of the extended portions of the IDTs touch one another.
6. The acoustofluidic contactless sample agitator of any one of aspects 4-5, wherein the extended portions of the IDTs are covered with a shielding material.
7. The acoustofluidic contactless sample agitator of aspect 6, wherein the shielding material is a self-assembled monolayer film of trichlorosilane, silicon dioxide, or Teflon film.
8. The acoustofluidic contactless sample agitator of any one of aspects 1-7, wherein the piezoelectric substrate is a is a (a) piezoelectric ceramic, optionally barium titanate, lead titanate, lead zirconate titanate, potassium niobate, lithium niobate, sodium tungstate, (b) a piezoelectric crystal, optionally quartz, topaz, tourmaline, berlinite, gallium orthophosphate, langasite, (c) a piezoelectric thin film, optionally zinc oxide, aluminum nitride, or (d) any combination of (a)-(c).
9. The acoustofluidic contactless sample agitator of any one of the aspects 1-7, wherein the IDTs are planar IDTs.
10. The acoustofluidic contactless sample agitator of any one of aspects 1-9, wherein the pair of IDTs forming an x-axis pair of the two pairs of orthogonal IDTs has a different configuration than the pair of IDTs forming a y-axis pair of the two pairs of orthogonal IDTs.
11. The acoustofluidic contactless sample agitator of aspect 10, wherein the number of interleaving electrodes in each IDT of the x-axis pair of IDTs is the same, wherein the number of interleaving electrodes in each IDT of the y-axis pair of IDTs is the same, and wherein the number of interleaving electrodes in the each of the x-axis pair of IDTs is different than the number of interleaving electrodes in the y-axis pair of IDTs.
12. The acoustofluidic contactless sample agitator of any one of aspects 10-11, wherein the number of interleaving electrodes in each of the x-axis pair of IDTs is less than, is more than, or is equal to the number of interleaving electrodes in each of the y-axis pair of IDTs.
13. The acoustofluidic contactless sample agitator of any one of aspects 10-12, wherein the number of interleaving electrodes in each of the x-axis pair of IDTs and/or the number of interleaving electrodes in each of the y-axis pair of IDTs ranges from 1-100, 5-100, 10-100, 15-100, 20-100, 25-100, 30-100, 35-100, 40-100, 45-100, 50-100, 55-100, 60-100, 65-100, 70-100, 75-100, 80-100, 85-100, 90-100, or 95-100.
14. The acoustofluidic contactless sample agitator of any one of aspects 10-13, wherein the number of interleaving electrodes in each of the y-axis pair of IDTs is 20 and/or the number of interleaving electrodes in each of the x-axis pair of IDTs is 5.
15. The acoustofluidic contactless sample agitator of any one of aspects 10-14, wherein the spacing between each interleaving electrode of each IDT in the x-axis pair of IDTs is different than or is the same as the spacing between each interleaving electrode of each IDT in the y-axis pair of IDTs, and optionally wherein the spacing between each interleaving electrode of each IDT in the x-axis pair, the y-axis pair, or both ranges from about 10 microns to about 150 microns, about 20 microns to about 150 microns, about 30 microns to about 150 microns, about 40 microns to about 150 microns, about 50 microns to about 150 microns, about 60 microns to about 150 microns, about 70 microns to about 150 microns, about 80 microns to about 150 microns, about 90 microns to about 150 microns, about 100 microns to about 150 microns, about 110 microns to about 150 microns, about 120 microns to about 150 microns, about 130 microns to about 150 microns, or about 140 microns to about 150 microns.
16. The acoustofluidic contactless sample agitator of aspect 15, wherein the spacing of each IDT in the x-axis pair of IDTs is about 5 microns, the spacing of each IDT in the y-axis pair is about 20 microns, or both.
17. The acoustofluidic contactless sample agitator of claim 1, wherein the aperture is or ranges from about 10λ, 20λ, 30λ, 40λ, 50λ, 60λ, 70λ, 80λ, 90λ, 100λ, 110λ, 120λ, 130λ, 140λ, 150λ, 160λ, 170λ, 180λ, 190λ, 200λ, 210, 220λ, 230λ, 240λ, 250λ, 260λ, 270λ, 280λ, 290λ, 300λ, 310λ, 320λ, 330λ, 340λ, 350λ, 360λ, 370λ, 380λ, 390λ, 400λ, 410λ, 420λ, 430λ, 440λ, 450λ, 460λ, 470λ, 480λ, 490λ, 500λ, 510λ, 520λ, 530λ, 540λ, 550λ, 560λ, 570λ, 580λ, 590λ, 600λ, 610λ, 620λ, 630λ, 640λ, 650λ, 660λ, 670λ, 680λ, 690λ, 700λ, 710λ, 720λ, 730λ, 740λ, 750λ, 760λ, 770λ, 780λ, 790λ, 800λ, 810λ, 820λ, 830λ, 840λ, 850λ, 860λ, 870, 880λ, 890λ, 900λ, 910λ, 920λ, 930λ, 940λ, 950λ, 960λ, 970λ, 980λ, 990λ or/to about 1000λ.
18. The acoustofluidic contactless sample agitator of any one of aspects 10-17, wherein the width and/or length of each interleaving electrode of each IDT in the x-axis pair of IDTs is different than or is the same as the width and/or length of each interleaving electrode of each IDT in the y-axis pair of IDTs.
19. The acoustofluidic contactless sample agitator of any one of aspects 1-18, wherein the acoustofluidic contactless sample agitator further comprises one or more reflector gratings, wherein one or more of the one or more reflector gratings backs one or more IDTs.
20. The acoustofluidic contactless sample agitator of aspect 19, wherein the acoustofluidic contactless sample agitator comprises two reflector gratings and wherein each of the two reflector gratings back a different IDT.
21. The acoustofluidic contactless sample agitator of aspect 20, wherein each of the two reflector gratings back a different IDT in the same pair of orthogonal IDTs.
22. The acoustofluidic contactless sample agitator of any one of aspects 20-21, wherein the reflector gratings back each IDT in the orthogonal pair of IDTs forming an x-axis pair of IDTs.
23. The acoustofluidic contactless sample agitator of any one of aspects 19-22, wherein the one or more reflector gratings comprise grating transducers comprising shortened electrodes, deposits of material on the piezoelectric material, such as periodic metal or other material strips deposited on the surface of the piezoelectric material.
24. The acoustofluidic contactless sample agitator of any one of aspects 19-23, wherein the reflector gratings each have a reflection coefficient ranging effective to achieve unidirectional wave propagation.
25. The acoustofluidic contactless sample agitator of any one of aspects 1-24, wherein the acoustofluidic contactless sample agitator is configured to generate ultrasonic surface acoustic waves with frequencies within the therapeutic or diagnostic imaging range.
26. The acoustofluidic contactless sample agitator of any one of aspects 1-25, wherein the acoustofluidic contactless sample agitator is configured to generate ultrasonic surface acoustic waves with frequencies that are or are ranging from about 2 MHz to about 200 MHz or more, optionally from about 5 MHz, 10 MHz, 20 MHz, 30 MHz, 40 MHz, 50 MHz, 60 MHz, 70 MHz, 80 MHz, 90 MHz, 100 MHz, 110 MHz, 120 MHz, 130 MHz, 140 MHz, 150 MHz, 160 MHz, 170 MHz, 180 MHz, or about 190 MHz to about 200 MHz.
27. The acoustofluidic contactless sample agitator of any one of aspects 1-26, wherein the acoustofluidic contactless sample agitator is configured to generate and propagate surface acoustic waves having a cell scale wavelength.
28. The acoustofluidic contactless sample agitator of aspect 27, wherein the wavelength is less than the diameter of an oocyte and greater than 0.
29. The acoustofluidic contactless sample agitator of any one of aspects 27-28, wherein the wavelength is about the same as the average diameter of a cumulus cell or other cell complexed with or associated with an oocyte.
30. The acoustofluidc contactless sample agitator of aspect 29, wherein the wavelength is greater than zero and less than about 120 μm, 110 μm, 100 μm, 90 μm, 80 μm, 70 μm, 60 μm, 50 μm, 40 μm, 30 μm, 25 μm, 20 μm, 15 μm, 10 μm, or less than about 5 μm.
31. The acoustofluidic contactless sample agitator of aspect 30, wherein the wavelength ranges from about 5 μm to about 120 μm, optionally from about 5-10 μm, 10-20 μm, 20-30 am, 30-40 μm, 40-50 μm, 50-60 μm, 60-70 μm, 70-80 μm, 80-90 μm, 90-100 μm, 100-110 am, or 110-120 μm.
32. The acoustofluidic contactless sample agitator of any one of aspects 1-31, wherein the chamber comprises an outlet, an inlet, or both.
33. The acoustofluidic contactless sample agitator of any one of aspects 1-32, wherein the chamber is configured as a well, a microwell, a channel, or a microchannel.
34. The acoustofluidic contactless sample agitator of any one of aspects 1-33, wherein the chamber comprises a closed top.
35. The acoustofluidic contactless sample agitator of any one of aspects 1-33, wherein the chamber comprises an open top.
36. The acoustofluidic contactless sample agitator of any one of aspects 1-35, wherein the volume of a liquid is about 1-1000 nL, μL, or mL.
37. The acoustofluidic contactless sample agitator of any one of aspects 1-36, wherein the chamber is configured to fluidically coupled to one or more other chambers as in any of the preceding claims, a microchannel, and/or other microfluidic device.
38. The acoustofluidic contactless sample agitator of any one of aspects 1-37, wherein each IDT of each pair of the two pairs of orthogonal IDTs are electrically coupled to a printed circuit board.
39. The acoustofluidic contactless sample agitator of any one of aspects 1-38, wherein in the chamber is any suitable three-dimensional shape.
40. The acoustofluidic contactless sample agitator of any one of aspects 1-39, wherein the chamber comprises a tapered portion that begins at a position on the one or more walls and ends at the opening.
41. The acoustofluidic contactless sample agitator of any one of aspects 1-40, wherein the opening is any two-dimensional shape, optionally a circle, an ellipse, a rectangle, a square, a triangle, a regular polygon, or an irregular two-dimensional shape.
42. The acoustofluidic contactless sample agitator of any one of aspects 1-41, wherein the chamber comprises a coated or non-coated polymeric material, glass, a metal, fused silica, a ceramic, or any combination thereof, wherein the coated or non-coated polymeric material is optionally polydimethylsiloxane (PDMS), polystyrene, or polycarbonate.
43. The acoustofluidic contactless sample agitator of any one of aspects 1-42, wherein at least a surface of the chamber that comes in contact with the volume of liquid is biocompatible.
44. The acoustofluidic contactless sample agitator of any one of aspects 1-43, wherein one or more components of the acoustofluidic contactless sample agitator are made from an optically transparent or optically translucent material.
45. The acoustofluidic contactless sample agitator of any one of aspects 1-44, wherein the acoustofluidic contactless sample agitator is configured to propagate surface acoustic waves in one or more wave modalities, wherein the one or more wave modalities are optionally selected from continuous wave, frequency modulation, pulse modulation, swept frequency modulation, or any combination thereof.
46. The acoustofluidic contactless sample agitator of any one of aspects 1-45, wherein the acoustofluidic contactless sample agitator is configured to denudate cumulus-oocyte-complexes.
47. The acoustofluidic contactless sample agitator of any one of aspects 1-46, wherein the acoustofluidic contactless sample agitator is configured to achieve one or more patterns of acoustic streaming within the chamber.
48. The acoustofluidic contactless sample agitator of aspect 43, wherein the pattern of acoustic streaming is horizontal vortices, vertical vortices, chaotic azimuthal recirculations, poloidal flow, toroidal circulations, or any combination thereof.
49. The acoustofluidic contactless sample agitator of any one of aspects 1-48, wherein the acoustofluidic contactless sample agitator is configured to agitate one or more cells, particles, and/or complexes thereof present in a volume of liquid present in the chamber without imparting significant damage or result in a significant loss of one or more functions in the majority of one or more types of cells and/or particles present in the volume of liquid.
50. The acoustofluidic contactless sample agitator of any one of aspect 45, wherein the one or more functions is viability and/or embryo development potential.
51. The acoustofluidic contactless sample agitator of aspects 1-50, wherein the acoustofluidic contactless sample agitator is configured to agitate one or more complexes of cells present in a volume of liquid present in the chamber such that one or more cells and/or cell types present in the one or more complexes of cells are separated from the one or more complexes of cells.
52. A microfluidic device comprising one or more of the acoustofluidic contactless sample agitator of any one of aspects 1-51.
53. The microfluidic device of aspect 52, wherein the microfluidic device comprises a plurality acoustofluidic contactless sample agitators as in any one of aspects 1-51.
54. The microfluidic device of any one of aspects 52-53, wherein the microfluidic device is configured as a chip.
55. The microfluidic device of any one of aspects 52-54, wherein the microfluidic device or component thereof is configured to be or is fluidically coupled with, electrically coupled with, and/or is in fluidic, electrical, optical, and/or wireless communication with one or more other devices and/or systems capable of upstream or downstream processing of one or more cells present in a volume of a liquid present in the chamber.
56. The microfluidic device of any one of aspects 52-55, wherein the microfluidic device is configured for automatic agitation of one or more liquid samples present in one or more chambers.
57. A sample processing system comprising:
   a. an acoustofluidic device as in any one of aspects 1-51 or a microfluidic device as in any one of aspects 52-56;
   b. one or more additional devices and/or systems capable of additional upstream or downstream processing, analyzing, manipulating, and/or storage of one or more particles, cells, and/or complexes thereof in the sample, wherein the one or more additional devices and/or systems are fluidically coupled with, electrically coupled with, and/or is in fluidic, electrical, optical, and/or wireless communication the acoustofluidic device or microfluidic device of (a).
58. The sample processing system of aspect 57, wherein the system is configured for oocyte processing and/or intracytoplasmic sperm injection.
59. The sample processing system of any one of aspects 57-58, wherein the one or more additional devices and/or systems is one or more of the following:
   a. an oocyte sorter;
   b. an oocyte immobilizing station;
   c. a spermatozoa reservoir;
   d. a spermatozoa sorter;
   e. a motile spermatozoa immobilization station;
   f. an injector;
   g. an embryo culturing chamber;
   h. one or more cell imaging devices;
   i. one or more system processors and/or controllers;
   j. one or more media reservoirs;
   k. a cell manipulation station;
   l. a spermatozoa storage chamber;
   m. an oocyte storage chamber; or
   n. any combination thereof.
60. The system of any one of aspects 57-59, wherein the system or one or more devices or systems thereof are automated.
61. A method of separating cells from a complex of cells comprising:
   exposing a sample comprising complexes of cells present in the chamber of an acoustofluidic device as in any one of aspects 1-51, a microfluidic device as in any one of aspects 52-56, or a system of any one of aspects 57-6, to acoustic streaming and/or acoustic radiation force in the chamber by applying tunable orthogonal surface acoustic waves produced by the acoustofluidic device to the chamber, wherein the acoustic streaming produced in the chamber separates one or more cells and/or cell types from the complex of cells.
62. The method of aspect 61, wherein the wavelength of the surface acoustic waves is about the average diameter of the one or more cells or cell types to be separated from the complex of cells.
63. The method of any one of aspects 61-62, wherein the acoustic streaming produced in the chamber is sufficient to denudate one or more cells from one cell in the complex of cells.
64. The method of any one of aspects 61-63, wherein the complex of cells is a cumulus-oocyte-complex.
65. The method of aspect 64, wherein the wavelength ranges from about 5 μm to about 120 m, optionally about 5-10 μm, 10-20 μm, 20-30 μm, 30-40 μm, 40-50 μm, 50-60 μm, 60-70 μm, 70-80 μm, 80-90 μm, 90-100 μm, 100-110 μm, or 110-120 μm.
66. The method of any one of aspects 61-65, wherein the frequency of the surface acoustic wave ranges from about 2 MHz to about 200 MHz or more, optionally from about 5 MHz, 10 MHz, 20 MHz, 30 MHz, 40 MHz, 50 MHz, 60 MHz, 70 MHz, 80 MHz, 90 MHz, 100 MHz, 110 MHz, 120 MHz, 130 MHz, 140 MHz, 150 MHz, 160 MHz, 170 MHz, 180 MHz, or 190 MHz to about 200 MHz.
67. The method of any one of aspects 61-66, wherein cumulus cells are separated from an oocyte.
68. The method of any one of aspects 61-67, further comprising removing a separated cell from the chamber.
69. The method of aspect 68, further comprising processing, manipulating, analyzing, injecting, culturing, storing, or any combination thereof the removed separated cell, optionally using one or more a downstream devices and/or systems.
70. The method of aspect 69, wherein the one or more downstream devices and/or systems is/are fluidically coupled with, electrically coupled with, and/or is in fluidic, electrical, optical, and/or wireless communication the acoustofluidic device.
71. The method of any one of aspects 61-70, wherein one or more steps are automated.

What is claimed is:

1. An acoustofluidic contactless sample agitator comprising:
a piezoelectric substrate capable of propagating a surface acoustic wave;
a chamber configured to contain a volume of a liquid, the chamber comprising one or more walls forming the sides of the chamber and a bottom comprising an opening;
at least two pairs of orthogonal interdigitated transducers (IDTs) deposited on a surface of the piezoelectric substrate; and
wherein the piezoelectric substrate is coupled to the chamber such that the two pairs of orthogonal IDTs are arranged about the periphery of the opening on the bottom of the chamber such that each IDT in each IDT pair is opposite each other on different sides of the opening, that the two IDT pairs are substantially perpendicular to each other about the periphery of the opening, and that the opening is substantially centered between the two IDT pairs, and
wherein, together, the two pairs of IDTs are configured to produce tunable orthogonal surface acoustic waves in the piezoelectric substrate effective to produce acoustic streaming and/or acoustic radiation force within a liquid present in the chamber sufficient to mix the liquid present inside the chamber and/or agitate one or more particles, cells, and/or complexes thereof present in the liquid without trapping the one or more particles, cells, and/or complexes thereof within the liquid.

2. The acoustofluidic contactless sample agitator of claim 1, wherein chamber is detachably coupled to the piezoelectric substrate.

3. The acoustofluidic contactless sample agitator of claim 1, wherein the two pairs of orthogonal IDTs are arranged about the periphery of the opening such that they do not extend into the opening and do not contact a liquid present in the chamber.

4. The acoustofluidic contactless sample agitator of claim 1, wherein the two pairs of orthogonal IDTs are arranged about the periphery of the opening such that a portion of each IDT extends into the opening such that the portion contacts a liquid present in the chamber.

5. The acoustofluidic contactless sample agitator of claim 4, wherein none of the extended portions of the IDTs touch one another.

6. The acoustofluidic contactless sample agitator of claim 4, wherein the extended portions of the IDTs are covered with a shielding material.

7. The acoustofluidic contactless sample agitator of claim 6, wherein the shielding material is a self-assembled monolayer film of trichlorosilane, silicon dioxide, or Teflon film.

8. The acoustofluidic contactless sample agitator of claim 1, wherein the piezoelectric substrate is a (a) piezoelectric ceramic, optionally barium titanate, lead titanate, lead zirconate titanate, potassium niobate, lithium niobate, sodium tungstate, (b) a piezoelectric crystal, optionally quartz, topaz, tourmaline, berlinite, gallium orthophosphate, langasite, (c) a piezoelectric thin film, optionally zinc oxide, aluminum nitride, or (d) any combination of (a)-(c).

9. The acoustofluidic contactless sample agitator of claim 1, wherein the IDTs are planar IDTs.

10. The acoustofluidic contactless sample agitator of claim 1, wherein the pair of IDTs forming an x-axis pair of the two pairs of orthogonal IDTs has a different configuration than the pair of IDTs forming a y-axis pair of the two pairs of orthogonal IDTs.

11. The acoustofluidic contactless sample agitator of claim 10, wherein the number of interleaving electrodes in each IDT of the x-axis pair of IDTs is the same, wherein the number of interleaving electrodes in each IDT of the y-axis pair of IDTs is the same, and wherein the number of interleaving electrodes in the each of the x-axis pair of IDTs is different than the number of interleaving electrodes in the y-axis pair of IDTs.

12. The acoustofluidic contactless sample agitator of claim 10, wherein the number of interleaving electrodes in each of the x-axis pair of IDTs is less than, is more than, or is equal to the number of interleaving electrodes in each of the y-axis pair of IDTs.

13. The acoustofluidic contactless sample agitator of claim 10, wherein the number of interleaving electrodes in each of the x-axis pair of IDTs and/or the number of interleaving electrodes in each of the y-axis pair of IDTs ranges from 1-100, 5-100, 10-100, 15-100, 20-100, 25-100, 30-100, 35-100, 40-100, 45-100, 50-100, 55-100, 60-100, 65-100, 70-100, 75-100, 80-100, 85-100, 90-100, or 95-100.

14. The acoustofluidic contactless sample agitator of claim 10, wherein the number of interleaving electrodes in each of the y-axis pair of IDTs is 20 and/or the number of interleaving electrodes in each of the x-axis pair of IDTs is 5.

15. The acoustofluidic contactless sample agitator of claim 10, wherein the spacing between each interleaving electrode of each IDT in the x-axis pair of IDTs is different than or is the same as the spacing between each interleaving electrode of each IDT in the y-axis pair of IDTs, and optionally wherein the spacing between each interleaving electrode of each IDT in the x-axis pair, the y-axis pair, or both ranges from about 10 microns to about 150 microns, about 20 microns to about 150 microns, about 30 microns to about 150 microns, about 40 microns to about 150 microns, about 50 microns to about 150 microns, about 60 microns to about 150 microns, about 70 microns to about 150 microns, about 80 microns to about 150 microns, about 90 microns to about 150 microns, about 100 microns to about 150 microns, about 110 microns to about 150 microns, about 120 microns to about 150 microns, about 130 microns to about 150 microns, or about 140 microns to about 150 microns.

16. The acoustofluidic contactless sample agitator of claim 15, wherein the spacing of each IDT in the x-axis pair of IDTs is about 5 microns, the spacing of each IDT in the y-axis pair is about 20 microns, or both.

17. The acoustofluidic contactless sample agitator of claim 1, wherein the aperture is or ranges from about 10λ, 20λ, 30λ, 40λ, 50λ, 60λ, 70λ, 80λ, 90λ, 100λ, 110λ, 120λ, 130λ, 140λ, 150λ, 160λ, 170λ, 180λ, 190λ, 200λ, 210, 220λ, 230λ, 240λ, 250λ, 260λ, 270λ, 280λ, 290λ, 300λ, 310λ, 320λ, 330λ, 340λ, 350λ, 360λ, 370λ, 380λ, 390λ, 400λ, 410λ, 420λ, 430λ, 440λ, 450λ, 460λ, 470λ, 480λ, 490λ, 500λ, 510λ, 520λ, 530λ, 540λ, 550λ, 560λ, 570λ, 580λ, 590λ, 600λ, 610λ, 620λ, 630λ, 640λ, 650λ, 660λ, 670λ, 680λ, 690λ, 700λ, 710λ, 720λ, 730λ, 740λ, 750λ, 760λ, 770λ, 780λ, 790λ, 800λ, 810λ, 820λ, 830λ, 840λ, 850λ, 860λ, 870, 880λ, 890λ, 900λ, 910λ, 920λ, 930λ, 940λ, 950λ, 960λ, 970λ, 980λ, 990λ, or/to about 1000λ.

18. The acoustofluidic contactless sample agitator of claim 10, wherein the width, length, or both of each interleaving electrode of each IDT in the x-axis pair of IDTs is different than or is the same as the width and/or length of each interleaving electrode of each IDT in the y-axis pair of IDTs.

19. The acoustofluidic contactless sample agitator of claim 1, wherein the acoustofluidic contactless sample agitator further comprises one or more reflector gratings, wherein one or more of the one or more reflector gratings backs one or more IDTs.

20. The acoustofluidic contactless sample agitator of claim 19, wherein the acoustofluidic contactless sample agitator comprises two reflector gratings and wherein each of the two reflector gratings back a different IDT.

21. The acoustofluidic contactless sample agitator of claim 20, wherein each of the two reflector gratings back a different IDT in the same pair of orthogonal IDTs.

22. The acoustofluidic contactless sample agitator of claim 20, wherein the reflector gratings back each IDT in the orthogonal pair of IDTs forming an x-axis pair of IDTs.

23. The acoustofluidic contactless sample agitator of claim 19, wherein the one or more reflector gratings comprise grating transducers comprising shortened electrodes, deposits of material on the piezoelectric material, such as periodic metal or other material strips deposited on the surface of the piezoelectric material.

24. The acoustofluidic contactless sample agitator of claim 19, wherein the reflector gratings each have a reflection coefficient ranging effective to achieve unidirectional wave propagation.

25. The acoustofluidic contactless sample agitator of claim 1, wherein the acoustofluidic contactless sample agitator is configured to generate ultrasonic surface acoustic waves with frequencies within the therapeutic or diagnostic imaging range.

26. The acoustofluidic contactless sample agitator of claim 1, wherein the acoustofluidic contactless sample agitator is configured to generate ultrasonic surface acoustic waves with frequencies that are or are ranging from about 2 MHz to about 200 MHz or more, optionally from about 5 MHz, 10 MHz, 20 MHz, 30 MHz, 40 MHz, 50 MHz, 60 MHz, 70 MHz, 80 MHz, 90 MHz, 100 MHz, 110 MHz, 120 MHz, 130 MHz, 140 MHz, 150 MHz, 160 MHz, 170 MHz, 180 MHz, or about 190 MHz to about 200 MHz.

27. The acoustofluidic contactless sample agitator of any one of claims 1-26, wherein the acoustofluidic contactless sample agitator is configured to generate and propagate surface acoustic waves having a cell scale wavelength.

28. The acoustofluidic contactless sample agitator of claim 27, wherein the wavelength is less than the diameter of an oocyte and greater than 0.

29. The acoustofluidic contactless sample agitator of claim 27, wherein the wavelength is about the same as the average diameter of a cumulus cell or other cell complexed with or associated with an oocyte.

30. The acoustofluidc contactless sample agitator of claim 29, wherein the wavelength is greater than zero and less than about 120 μm, 110 μm, 100 μm, 90 μm, 80 μm, 70 μm, 60 μm, 50 μm, 40 μm, 30 μm, 25 μm, 20 μm, 15 μm, 10 μm, or less than about 5 μm.

31. The acoustofluidic contactless sample agitator of claim 30, wherein the wavelength ranges from about 5 μm to about 120 μm, optionally from about 5-10 μm, 10-20 μm, 20-30 μm, 30-40 μm, 40-50 μm, 50-60 μm, 60-70 μm, 70-80 μm, 80-90 μm, 90-100 μm, 100-110 μm, or 110-120 μm.

32. The acoustofluidic contactless sample agitator of claim 1, wherein the chamber comprises an outlet, an inlet, or both.

33. The acoustofluidic contactless sample agitator of claim 1, wherein the chamber is configured as a well, a microwell, a channel, or a microchannel.

34. The acoustofluidic contactless sample agitator of claim 1, wherein the chamber comprises a closed top.

35. The acoustofluidic contactless sample agitator of any one of claims 1-33, wherein the chamber comprises an open top.

36. The acoustofluidic contactless sample agitator of claim 1, wherein the volume of a liquid is about 1-1000 nL, μL, or mL.

37. The acoustofluidic contactless sample agitator of claim 1, wherein the chamber is configured to fluidically coupled to one or more other chambers as in any of the preceding claims, a microchannel, and/or other microfluidic device.

38. The acoustofluidic contactless sample agitator of claim 1, wherein each IDT of each pair of the two pairs of orthogonal IDTs are electrically coupled to a printed circuit board.

39. The acoustofluidic contactless sample agitator of claim 1, wherein in the chamber is any suitable three-dimensional shape.

40. The acoustofluidic contactless sample agitator of claim 1, wherein the chamber comprises a tapered portion that begins at a position on the one or more walls and ends at the opening.

41. The acoustofluidic contactless sample agitator of claim 1, wherein the opening is any two-dimensional shape, optionally a circle, an ellipse, a rectangle, a square, a triangle, a regular polygon, or an irregular two-dimensional shape.

42. The acoustofluidic contactless sample agitator of claim 1, wherein the chamber comprises a coated or non-coated polymeric material, glass, a metal, fused silica, a ceramic, or any combination thereof, wherein the coated or non-coated polymeric material is optionally polydimethylsiloxane (PDMS), polystyrene, or polycarbonate.

43. The acoustofluidic contactless sample agitator of claim 1, wherein at least a surface of the chamber that comes in contact with the volume of liquid is biocompatible.

44. The acoustofluidic contactless sample agitator of claim 1, wherein one or more components of the acoustofluidic contactless sample agitator are made from an optically transparent or optically translucent material.

45. The acoustofluidic contactless sample agitator of claim 1, wherein the acoustofluidic contactless sample agitator is configured to propagate surface acoustic waves in one or more wave modalities, wherein the one or more wave modalities are optionally selected from continuous wave, frequency modulation, pulse modulation, swept frequency modulation, or any combination thereof.

46. The acoustofluidic contactless sample agitator of claim 1, wherein the acoustofluidic contactless sample agitator is configured to denudate cumulus-oocyte-complexes.

47. The acoustofluidic contactless sample agitator of claim 1, wherein the acoustofluidic contactless sample agitator is configured to achieve one or more patterns of acoustic streaming within the chamber.

48. The acoustofluidic contactless sample agitator of claim 43, wherein the pattern of acoustic streaming is horizontal vortices, vertical vortices, chaotic azimuthal recirculations, poloidal flow, toroidal circulations, or any combination thereof.

49. The acoustofluidic contactless sample agitator of claim 1, wherein the acoustofluidic contactless sample agitator is configured to agitate one or more cells, particles, and/or complexes thereof present in a volume of liquid present in the chamber without imparting significant damage or result in a significant loss of one or more functions in the majority of one or more types of cells and/or particles present in the volume of liquid.

50. The acoustofluidic contactless sample agitator of any one of claim 45, wherein the one or more functions is viability and/or embryo development potential.

51. The acoustofluidic contactless sample agitator of claim 1, wherein the acoustofluidic contactless sample agitator is configured to agitate one or more complexes of cells present in a volume of liquid present in the chamber such that one or more cells and/or cell types present in the one or more complexes of cells are separated from the one or more complexes of cells.

52. A microfluidic device comprising one or more of the acoustofluidic contactless sample agitator of claim 1.

53. The microfluidic device of claim 52, wherein the microfluidic device comprises a plurality acoustofluidic contactless sample agitators according to claim 1.

54. The microfluidic device of claim 52, wherein the microfluidic device is configured as a chip.

55. The microfluidic device of claim 52, wherein the microfluidic device or component thereof is configured to be or is fluidically coupled with, electrically coupled with, and/or is in fluidic, electrical, optical, and/or wireless communication with one or more other devices and/or systems capable of upstream or downstream processing of one or more cells present in a volume of a liquid present in the chamber.

56. The microfluidic device of claim 52, wherein the microfluidic device is configured for automatic agitation of one or more liquid samples present in one or more chambers.

57. A sample processing system comprising:
a. an acoustofluidic device of claim 51 or a microfluidic device comprising the acoustofluidic device;
b. one or more additional devices and/or systems capable of additional upstream or downstream processing, analyzing, manipulating, and/or storage of one or more particles, cells, and/or complexes thereof in the sample, wherein the one or more additional devices and/or systems are fluidically coupled with, electrically coupled with, and/or is in fluidic, electrical, optical, and/or wireless communication the acoustofluidic device or microfluidic device of (a).

58. The sample processing system of claim 57, wherein the system is configured for oocyte processing and/or intracytoplasmic sperm injection.

59. The sample processing system of claim 57, wherein the one or more additional devices and/or systems is one or more of the following:
a. an oocyte sorter;
b. an oocyte immobilizing station;
c. a spermatozoa reservoir;
d. a spermatozoa sorter;
e. a motile spermatozoa immobilization station;
f. an injector;
g. an embryo culturing chamber;
h. one or more cell imaging devices;
i. one or more system processors and/or controllers;
j. one or more media reservoirs;
k. a cell manipulation station;
l. a spermatozoa storage chamber;
m. an oocyte storage chamber; or
n. any combination of (a)-(m).

60. The system of claim 57, wherein the system or one or more devices or systems thereof are automated.

61. A method of separating cells from a complex of cells comprising:
exposing a sample comprising complexes of cells present in the chamber of an acoustofluidic device of claim 1, a microfluidic device as in claim 52, or a system of claim 57 to acoustic streaming and/or acoustic radiation force in the chamber by applying tunable orthogonal surface acoustic waves produced by the acoustofluidic device to the chamber, wherein the acoustic streaming produced in the chamber separates one or more cells and/or cell types from the complex of cells.

62. The method of claim 61, wherein the wavelength of the surface acoustic waves is about the average diameter of the one or more cells or cell types to be separated from the complex of cells.

63. The method of claim 61, wherein the acoustic streaming produced in the chamber is sufficient to denudate one or more cells from one cell in the complex of cells.

64. The method of claim 61, wherein the complex of cells is a cumulus-oocyte-complex.

65. The method of claim 64, wherein the wavelength ranges from about 5 μm to about 120 m, optionally about 5-10 μm, 10-20 μm, 20-30 μm, 30-40 μm, 40-50 μm, 50-60 μm, 60-70 μm, 70-80 μm, 80-90 μm, 90-100 μm, 100-110 μm, or 110-120 μm.

66. The method of claim 61, wherein the frequency of the surface acoustic wave ranges from about 2 MHz to about 200 MHz or more, optionally from about 5 MHz, 10 MHz, 20 MHz, 30 MHz, 40 MHz, 50 MHz, 60 MHz, 70 MHz, 80 MHz, 90 MHz, 100 MHz, 110 MHz, 120 MHz, 130 MHz, 140 MHz, 150 MHz, 160 MHz, 170 MHz, 180 MHz, or 190 MHz to about 200 MHz.

67. The method of claim 61, wherein cumulus cells are separated from an oocyte.

68. The method of claim 61, further comprising removing a separated cell from the chamber.

69. The method of claim 68, further comprising processing, manipulating, analyzing, injecting, culturing, storing, or any combination thereof the removed separated cell, optionally using one or more a downstream devices and/or systems.

70. The method of claim 69, wherein the one or more downstream devices and/or systems is/are fluidically coupled with, electrically coupled with, and/or is in fluidic, electrical, optical, and/or wireless communication the acoustofluidic device.

71. The method of claim 61, wherein one or more steps are automated.
